(19) United States
(12) Patent Application Publication
(10) Pub. No.: US 2024/0335547 A1
(43) Pub. Date: Oct. 10, 2024

(54) MODIFIED CHOLIC ACID CONJUGATES

(71) Applicant: Ultra, LLC, Walnut Creek, CA (US)

(72) Inventors: Elias Shaheen, Walnut Creek, CA (US); Dan Draney, Walnut Creek, CA (US); Sharon Bonner-Brown, Walnut, CA (US)

(73) Assignee: Ultra, LLC, Walnut Creek, CA (US)

(21) Appl. No.: 18/745,512

(22) Filed: Jun. 17, 2024

Related U.S. Application Data
(63) Continuation of application No. PCT/US2023/032261, filed on Sep. 8, 2023.
(60) Provisional application No. 63/406,667, filed on Sep. 14, 2022.

(51) Int. Cl. A61K 47/54; A61K 47/55; A61P 31/04; A61P 31/10; A61P 31/12

(52) U.S. Cl. CPC A61K 47/554; A61K 47/551; A61P 31/04; A61P 31/10; A61P 31/12

(57) ABSTRACT

The present disclosure provides compounds, compositions, and methods, wherein the compounds include a modified cholic acid or a cholic acid component and at least a second component in which the second component is conjugated to the modified cholic acid or cholic acid having the following structure:

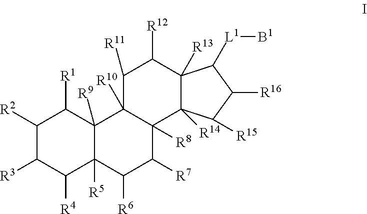

In certain instances, the conjugate compound provides reduced cytotoxicity, greater efficacy, and a more robust MOA (e.g., the entry into a cell is more facile or multiple modes of action occur) compared to the non-conjugated component alone. In certain instances, the second component such as biotin is covalently linked to the modified cholic acid or cholic acid.

### CROSS-REFERENCE TO RELATED APPLICATIONS

The current application is a continuation of PCT/US2023/032261, filed Sep. 8, 2023, which application claims priority to U.S. Provisional Patent Application No. 63/406,667, filed Sep. 14, 2022, the contents of which are hereby incorporated by reference in their entireties for all purposes.

### BACKGROUND

Broad spectrum antimicrobial agents are compounds that treat an array of bacteria including both Gram-positive and Gram-negative bacteria and a wide range of infections. Antimicrobial agents that treat bacteria, viruses and fungi are rare.

Cholic acid is synthesized in the liver from cholesterol and is a primary bile acid. Cholic acid is hydroxylated at C3, C7, and C12 and facilitates fat absorption and cholesterol excretion. Cholic acid, formulated as Cholbam capsules, is approved by the United States FDA for treatment for children and adults for treatment of bile acid synthesis disorders due to single enzyme defects.

WO 2018232502 teaches transfection reagents for delivery of nucleic acids comprising a lipid such as cholic acid or deoxycholic acid conjugated to a polymer such as polyethylenimine having a molecular weight up to 2.0 kDa.

US 20180050048 teaches conjugate compounds having at least one of a moiety derived from for example, ursodeoxycholic acid and a moiety derived from berberine or L-carnitine or metformin, etc. The application relates to pharmaceutical compositions, methods of preparation and use of these conjugates in treating various disorders, diabetes, diabetic complications, dyslipidemia, obesity, metabolic syndromes, and other indications.

US 20050239204 teaches a multifunctional molecular complex for the transfer of a nucleic acid composition to a target cell comprising 1) a nucleic acid composition; 2) one or more cationic polyamine components bound to said nucleic acid composition, each comprising from three to twelve nitrogen atoms; 3) one or more endosome membrane disruption promoting components, which include a) at least one lipophilic long chain alkyl group, b) a fusogenic peptide or c) cholic acid or cholesteryl or derivatives; and optionally 4) one or more receptor specific binding components which are ligands for natural receptors of the target cell.

In view of the foregoing, new broad spectrum antimicrobials, which are effective against bacteria, viruses and fungi are needed to explore and improve therapeutic effects for a range of purposes, such as to improve the stability of an aqueous solution of the drug, to trigger drug release for drug delivery, to improve drug bioavailability and body fluid circulation time, to avoid drug degradation failure as well as to improve efficacy. The present disclosure satisfies these needs and offer other advantages as well.

### BRIEF SUMMARY

The present disclosure provides compounds, compositions, and methods, wherein the compounds include a first component of cholic acid or a modified cholic acid (MCA) and a second component linked thereto, the second component comprising biotin, desthiobiotin or biotin mimetics. Accordingly, the present invention comprises cholic acid linked to biotin, desthiobiotin or biotin mimetics as well as modified cholic acid (MCA) similarly linked to biotin, desthiobiotin or biotin mimetics.

In certain instances, the conjugate compound provides reduced cytotoxicity in use relative to the use of the modified cholic acid component alone. In certain instances, the conjugate compounds possess a more robust mode of action (MOA) such as multiple modes of action, which facilitates entry into a cell. In certain instances, the second component is covalently bonded or linked to the modified cholic acid. In certain aspects, the second component is a biotin or a biotin moiety. In certain instances, the conjugate compounds provide comparable or superior efficacy in therapeutic treatments relative to treatments incorporating the unconjugated MCA.

As such, in one embodiment, the present disclosure provides a compound having the following formula I, or a pharmaceutically acceptable salt thereof:

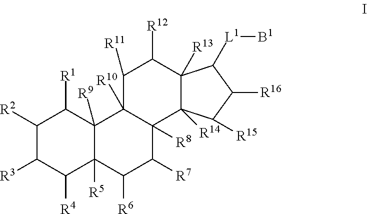

wherein:
R¹-R⁴, R⁶, R⁷, R¹¹, R¹², R¹⁵ and R¹⁶ is each independently a member selected from the group consisting of hydrogen, hydroxyl, a substituted or unsubstituted (C₁-C₁₀) alkyl, a substituted or unsubstituted (C₁-C₁₀) alkylcarboxy, (C₁-C₁₀) hydroxyalkyl, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, a substituted or unsubstituted aryl, (C₁-C₁₀) haloalkyl, C₁-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₉) aminoalkylaminocarbonyl, a substituted or unsubstituted (C₁-C₁₀) aminoalkylcarboxamido, (C₁-C₁₀) azidoalkyloxy, (C₁-C₁₀) cyanoalkyloxy, (C₁-C₁₀) guanidinoalkyl oxy, and (C₁-C₁₀) guanidinoalkyl carboxy;
R⁵, R⁸, R⁹, R¹⁰, R¹³, and R¹⁴ is each independently a member selected from the group consisting of hydrogen, hydroxyl, a substituted or unsubstituted (C₁-C₁₀) alkyl, (C₁-C₁₀) hydroxyalkyl, (C₁-C₁₀) alkyloxy-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) aminoalkylcarboxy, a substituted or uusubstituted (C₁-C₁₀) aminoalkylaminocarbonyl, (C₁-C₁₀) azidoalkyloxy, (C₁-C₁₀) cyanoalkyloxy, (C₁-C₁₀) guanidinoalkyloxy, and (C₁-C₁₀) guanidinoalkylcarboxy;
L¹ is a linking group; and
B¹ is biotin, biotin, desthiobiotin, or a biotin mimetic.

In another embodiment, the present disclosure provides a method for treating a microorganism infection or retarding the spread of the microorganism, the method comprising:
contacting the microorganism with an antimicrobial amount of a compound having formula I or a pharmaceutically acceptable salt thereof:

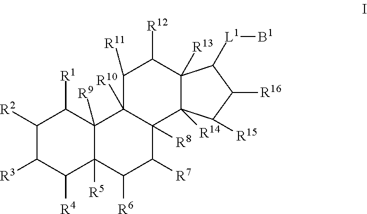

wherein:
R¹-R⁴, R⁶, R⁷, R¹¹, R¹², R¹⁵ and R¹⁶ is each independently a member selected from the group consisting of hydrogen, hydroxyl, a substituted or unsubstituted (C₁-C₁₀) alkyl, a substituted or unsubstituted (C₁-C₁₀) alkyl, a substituted or unsubstituted (C₁-C₁₀) alkylcarboxy, (C₁-C₁₀) hydroxyalkyl. (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, a substituted or unsubstituted (C₁-C₁₀) aminoalkylcarboxamido, (C₁-C₁₀) azidoalkyloxy, (C₁-C₁₀) cyanoalkyloxy, (C₁-C₁₀) guanidinoalkyl oxy, and (C₁-C₁₀) guanidinoalkyl carboxy; R⁵, R⁸, R⁹, R¹⁰, R¹³, and R¹⁴ is each independently a member selected from the group consisting of hydrogen, hydroxyl, a substituted or unsubstituted (C₁-C₁₀) alkyl, (C₁-C₁₀) hydroxyalkyl, (C₁-C₁₀) alkyloxy-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) aminoalkylcarboxy, a substituted or uusubstituted (C₁-C₁₀) aminoalkylaminocarbonyl, (C₁-C₁₀) azidoalkyloxy, (C₁-C₁₀) cyanoalkyloxy, (C₁-C₁₀) guanidinoalkyloxy, and (C₁-C₁₀) guanidinoalkylcarboxy;
L¹ is a linking group; and
B¹ is biotin or a biotin moiety, wherein the compound of formula I kills or slows the spread of the microorganism. In certain instances, the compounds of the disclosure act as immunomodulators, which can stimulate or suppress the immune system.

These and other aspects, object and embodiments will become more apparent when read with the figures and detailed description that follows.

### BRIEF DESCRIPTION OF THE DRAWINGS

FIG. 1 illustrates cell toxicity in human foreskin fibroblasts (hFFs) of various CSA-44 concentrations (a modified cholic acid).

FIG. 2 illustrates cell toxicity in human foreskin fibroblasts (hFFs) of an embodiment of the present disclosure at various concentrations.

FIG. 3 shows that an embodiment of the present disclosure has a IC₅₀ of 154 μM in Cal-u3 cells.

FIG. 4 illustrates protease inhibitory activity of an embodiment of the present disclosure.

FIGS. 5A-B illustrates the antiviral activity of an embodiment of the present disclosure. FIG. 5A shows the reduction of viral strain WA and FIG. 5B shows the reduction of viral strain BA.5 in the presence of a compound of this disclosure.

FIG. 6 illustrates the in vivo level of virus in lung tissue of a Control Group (PBS) versus a Group administered an embodiment of this disclosure.

FIGS. 7A-H illustrate imaging experiments with H&E staining. FIGS. 7A-B show lung tissues from the non-infected mice showing normal morphology; FIGS. 7C-D show non-infected tissues that are treated with an embodiment of the present disclosure, which shows normal lung morphology; FIGS. 7E-F show infected tissue treated with PBS, which exhibited structural changes in the lungs; FIGS. 7G-H show infected tissue treated with an embodiment of the present disclosure, which shows normal lung morphology.

FIG. 8A-B illustrate a comparison of cytokine levels between a control Group (infected, but not treated) with an infected Group treated with an embodiment of the present disclosure (FIG. 8A). FIG. 8B shows a comparison of cytokine levels between a non-infected Group and an infected Group treated with an embodiment of the present disclosure.

### DETAILED DESCRIPTION

### I. Definitions

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“Acyl” as used herein includes an alkanoyl, aroyl, heterocycloyl, or heteroaroyl group as defined herein. Representative acyl groups include acetyl, benzoyl, nicotinoyl, and the like.

“Alkanoyl” as used herein includes an alkyl-C(O)— group wherein the alkyl group is as defined herein. Representative alkanoyl groups include acetyl, ethanoyl, pentanoyl, hexanoyl, and the like.

“Alkenyl” as used herein includes a straight or branched aliphatic hydrocarbon group of 2 to about 15 carbon atoms that contains at least one carbon-carbon double or triple bond. Preferred alkenyl groups have 2 to about 12 carbon atoms. More preferred alkenyl groups contain 2 to about 6 carbon atoms. In one aspect, hydrocarbon groups that contain a carbon-carbon double bond are preferred. In a second aspect, hydrocarbon groups that contain a carbon-carbon triple bond are preferred (i.e., alkynyl). “Lower alkenyl” as used herein includes alkenyl of 2 to about 6 carbon atoms. Representative alkenyl groups include vinyl, allyl, n-butenyl, 2-butenyl, 3-methylbutenyl, n-pentenyl, heptenyl, octenyl, decenyl, propynyl, 2-butynyl, 3-methylbutynyl, n-pentynyl, heptynyl, and the like. An alkenyl group can be unsubstituted or optionally substituted. When optionally substituted, one or more hydrogen atoms of the alkenyl group (e.g., from 1 to 4, from 1 to 2, or 1) may be replaced with a moiety independently selected from the group of fluoro, hydroxy, alkoxy, amino, alkylamino, acylamino, thio, and alkylthio.

“Alkenylene” as used herein includes a straight or branched bivalent hydrocarbon chain containing at least one carbon-carbon double or triple bond. Preferred alkenylene groups include from 2 to about 12 carbons in the chain, and more preferred alkenylene groups include from 2 to 6 carbons in the chain. In one aspect, hydrocarbon groups that contain a carbon-carbon double bond are preferred. In a second aspect, hydrocarbon groups that contain a carbon-carbon triple bond are preferred. Representative alkenylene groups include —CH═CH—, —CH₂—CH═CH—, —C(CH₃)—CH—, —CH₂CH═CHCH₂—, ethynylene, propynylene, n-butynylene, and the like.

“Alkoxy” as used herein includes an alkyl-O— group wherein the alkyl group is as defined herein. Representative alkoxy groups include methoxy, ethoxy, n-propoxy, i-propoxy, n-butoxy, heptoxy, and the like. An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group of fluoro, hydroxy, alkoxy, amino, alkylamino, acylamino, thio, and alkylthio.

“Alkoxyalkyl” as used herein includes an alkyl-O-alkylene-group wherein alkyl and alkylene are as defined herein. Representative alkoxyalkyl groups include methoxyethyl, ethoxymethyl, n-butoxymethyl and cyclopentylmethyloxyethyl.

“Alkoxycarbonyl” as used herein includes an ester group; i.e., an alkyl-O—CO— group wherein alkyl is as defined herein. Representative alkoxycarbonyl groups include methoxycarbonyl, ethoxycarbonyl, t-butyloxycarbonyl, and the like.

“Alkoxycarbonylalkyl” as used herein includes an alkyl-O—CO-alkylene-group wherein alkyl and alkylene are as defined herein. Representative alkoxycarbonylalkyl include methoxycarbonylmethyl, ethoxycarbonylmethyl, methoxycarbonylethyl, and the like.

“Alkyl” as used herein includes an aliphatic hydrocarbon group, which may be straight or branched-chain, having about 1 to about 20 carbon atoms in the chain. Preferred alkyl groups have 1 to about 12 carbon atoms in the chain. More preferred alkyl groups have 1 to 6 carbon atoms in the chain. “Branched-chain” as used herein includes that one or more lower alkyl groups such as methyl, ethyl or propyl are attached to a linear alkyl chain. “Lower alkyl” as used herein includes 1 to about 6 carbon atoms, preferably 5 or 6 carbon atoms in the chain, which may be straight or branched. Representative alkyl groups include methyl, ethyl, n-propyl, isopropyl, n-butyl, t-butyl, n-pentyl, and 3-pentyl. In certain instances, a bond on the backbone such as “ [symbol] ” or “ [symbol] ” without an attached substituent, indicates a methyl group appended thereto.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group of halo such as chloro, fluoro, bromo or iodo, hydroxy, alkoxy, amino, alkylamino, acylamino, thio, and alkylthio. “Alkylene” as used herein includes a straight or branched bivalent hydrocarbon chain of 1 to about 6 carbon atoms. Preferred alkylene groups are the lower alkylene groups having 1 to about 4 carbon atoms. Representative alkylene groups include methylene, ethylene, and the like.

“Alkylamino” as used herein includes an alkyl-NRR′— group wherein the alkyl group is as defined herein. Alkylamino groups are those wherein the alkyl group is C₁-C₁₀ alkyl. Representative alkylamino groups include methylamino, ethylamino, isopropylamino, heptylamino, and the like. The amino group can be a substituted amino group. NRR′ group where R and R′ are members independently selected from the group of H and alkyl.

“Alkylcarboxy” and “alkylcarboxyl” as used herein include a RC(O)O— group (i.e., an ester). Representative alkylcarboxy groups include methylcarboxy, ethylcarboxy, t-butylcarboxy, and the like. In certain instances, the alkyl group is a lower alkyl group which includes 1 to about 6 carbon atoms, preferably 5 or 6 carbon atoms in the chain, which may be straight or branched. Representative alkyl groups include methyl, ethyl, n-propyl, isopropyl, n-butyl, t-butyl, n-pentyl, and 3-pentyl. The alkyl group can be substituted as defined herein.

“Alkylcarboxamido” as used herein includes an alkyl-C(O)—NR′R″— group wherein the alkyl group is as defined herein. Alkyl-C(O)—NRR′ groups are those wherein the alkyl group is C₁-C₁₀ alkyl. Representative alkyl-C(O)—NH— groups include methyl-C(O)-amino, ethyl-C(O)-amino, isopropyl-C(O)-amino, heptyl-C(O)-amino, and the like. NRR′ group where R and R′ are members independently selected from the group of H and C₁-C₁₀ alkyl. More preferably, at least one of R and R′ is H.

“Alkylheterocyclyl-alkylcarboxamido” as used herein includes an alkyl-heterocyclyl-alkyl-C(O)—NRR′ group. A heterocycyl group can be unsubstituted or optionally substituted. When optionally substituted, one or more hydrogen atoms of the group (e.g., from 1 to 4, from 1 to 2, or 1) may be replaced with a moiety independently selected from fluoro, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycyl group is unsubstituted or not optionally substituted. The heterocyclyl group can be aromatic or nonaromatic.

“Alkylthio” as used herein includes an alkyl-S— group wherein the alkyl group is as defined herein. Preferred alkylthio groups are those wherein the alkyl group is lower alkyl. Representative alkylthio groups include methylthio, ethylthio, isopropylthio, heptylthio, and the like.

“Alkylthioalkyl” as used herein includes an alkylthio-alkylene-group wherein alkylthio and alkylene are defined herein. Representative alkylthioalkyl groups include methylthiomethyl, ethylthiopropyl, isopropylthioethyl, and the like.

“Amido” as used herein includes a group of formula Y₁Y₂N—C(O)— wherein Y₁ and Y₂ are independently hydrogen, alkyl, or alkenyl; or Y₁ and Y₂, together with the nitrogen through which Y₁ and Y₂ are linked, join to form a 4- to 7-membered azaheterocyclyl group (e.g., piperidinyl). Representative amido groups include primary amido (H₂N—C(O)—), methylamido, dimethylamido, diethylamido, and the like. Preferably, “amido” is an —C(O)NRR′ group where R and R′ are members independently selected from the group of H and alkyl. More preferably, at least one of R and R′ is H.

“Amidoalkyl” as used herein includes an amido-alkylene-group wherein amido and alkylene are defined herein. Representative amidoalkyl groups include amidomethyl, amidoethylene, dimethylamidomethyl, and the like.

“Amino” as used herein includes a group of formula Y₁Y₂N— wherein Y₁ and Y₂ are independently hydrogen, acyl, or alkyl; or Y₁ and Y₂, together with the nitrogen through which Y₁ and Y₂ are linked, join to form a 4- to 7-membered azaheterocyclyl group (e.g., piperidinyl). Optionally, when Y₁ and Y₂ are independently hydrogen or alkyl, an additional substituent can be added to the nitrogen, making a quaternary ammonium ion.

Representative amino groups include primary amino (H₂N—), methylamino, dimethylamino, diethylamino, and the like. Preferably, “amino” is an —NRR′ group where R and R′ are members independently selected from the group of H and alkyl. Preferably, at least one of R and R′ is H. In certain instances, the amino group can be a cationic amine salt such as —NH₃⁺Cl⁻.

“Aminoalkyl” as used herein includes an amino-alkylene-group wherein amino and alkylene are defined herein. Representative aminoalkyl groups include aminomethyl, aminoethyl, dimethylaminomethyl, and the like.

“Aroyl” as used herein includes an aryl-CO— group wherein aryl is defined herein. Representative aroyl include benzoyl, naphth-1-oyl and naphth-2-oyl.

“Aroylalkyl as used herein includes an aryl-CO-alkyl group wherein aryl is defined herein. Representative aroylalkyl include benzoylalkyl, naphth-1-oyl-alkyl and naphth-2-oyl-alkyl.

“Aryl” as used herein includes an aromatic monocyclic or multicyclic ring system of 6 to about 14 carbon atoms, preferably of 6 to about 10 carbon atoms. Representative aryl groups include phenyl and naphthyl.

The term “arylalkyl” or “aralkyl” as used herein includes an alkyl group as defined herein where at least one hydrogen substituent has been replaced with an aryl group as defined herein. Examples include, but are not limited to, benzyl, 1-phenylethyl, 4-methylbenzyl, and 1,1,-dimethyl-1-phenylmethyl. A arylalkyl or aralkyl group can be unsubstituted or optionally substituted as per its component groups. For example, but without limitation, the aryl group of an arylalkyl group can be substituted, such as in 4-methylbenzyl, 2,4,6-trimethylbenzyl, 4-tert-butylbenzyl, 4-isopropylbenzyl, and the like. In some aspects, the group is unsubstituted or not optionally substituted, especially if including a defined substituent, such as a hydroxyalkyl or alkylaminoalkoxy group.

“Aromatic ring” as used herein includes 5-12 membered aromatic monocyclic or fused polycyclic moieties that may include from zero to four heteroatoms selected from the group of oxygen, sulfur, selenium, and nitrogen. Exemplary aromatic rings include benzene, pyrrole, furan, thiophene, imidazole, oxazole, thiazole, triazole, pyrazole, pyridine, pyrazine, pyridazine, pyrimidine, naphthalene, benzathiazoline, benzothiophene, benzofurans, indole, benzindole, quinoline, and the like. The aromatic ring group can be substituted at one or more positions with halo, alkyl, alkoxy, alkoxy carbonyl, haloalkyl, cyano, sulfonato, amino sulfonyl, aryl, sulfonyl, aminocarbonyl, carboxy, acylamino, alkyl sulfonyl, amino and substituted or unsubstituted substituents.

“B¹ or a biotin moiety” as used herein includes biotin (CAS 58-85-5), desthiobiotin (CAS 533-48-2) as well as biotin mimetics. Typically, a biotin mimetic is a peptide that binds streptavidin, or avidin or neutravidin. Avidin is a glycoprotein found in the egg white and tissues of birds, reptiles, and amphibia. The biotin-binding protein contains four identical subunits having a combined mass of 67,000-68,000 daltons. Removing the glycosylation from avidin yields Neutravidin Protein with a mass of 60,000 daltons. In certain instances, a biotin mimetic contains a His-Pro-Gln (HPQ) tripeptide or HPQN sequence (See, Devlin, J. J.; Panganiban, L. C.; Devlin, P. E. Random peptide libraries: A source of specific protein binding molecules. Science 1990, 249, 404-406 or Luo et al., 1998 J. Biotechnol. 65:225 and references cited therein). The biotin memetic may be about 3 to about 20 amino acids. In certain instances, a biotin mimetic is a moiety that binds a receptor that similarly binds biotin.

In certain instances, a biotin mimetic is any compound capable of binding to avidin or streptavidin.

“Carboxy” and “carboxyl” as used herein include a HOC(O)— group (i.e., a carboxylic acid) or a salt thereof.

“Carboxyalkyl” as used herein includes a HOC(O)-alkylene-group wherein alkylene is defined herein. Representative carboxyalkyls include carboxymethyl (i.e., HOC(O)CH₂—) and carboxyethyl (i.e., HOC(O)CH₂CH₂—).

A modified cholic acid (“MCA”) conjugate as used herein is a synthetically produced chemical compound that includes a backbone of cholic acid, chenodeoxycholic acid, deoxycholic acid or lithocholic acid, with A-D cyclic fused rings having formula I, wherein R¹ to R¹⁶, L¹ and B¹ are defined herein, which may comprise various charged groups (e.g., amines and cationic groups) attached to the backbone. A MCA conjugate has an appended B¹, which includes biotin biotin, desthiobiotin, as well as biotin mimetics that is covalently attached to the backbone via a linker. The MCA conjugates have antimicrobial attributes.

“Cycloalkyl” as used herein includes a non-aromatic mono- or multicyclic ring system of about 3 to about 10 carbon atoms, preferably of about 5 to about 10 carbon atoms. More preferred cycloalkyl rings contain 5 or 6 ring atoms. A cycloalkyl group optionally comprises at least one sp²-hybridized carbon (e.g., a ring incorporating an endocyclic or exocyclic olefin). Representative monocyclic cycloalkyl groups include cyclopentyl, cyclohexyl, cyclohexenyl, cycloheptyl, and the like. Representative multicyclic cycloalkyl include 1-decalin, norbornyl, adamantyl, and the like.

“Cycloalkylene” as used herein includes a bivalent cycloalkyl having about 4 to about 8 carbon atoms. Preferred cycloalkylenyl groups include 1,2-, 1,3-, or 1,4-cis- or trans-cyclooctylene.

The terms “disorder,” “disease,” and “condition” are used herein interchangeably for a condition in a subject. A disorder is a disturbance or derangement that affects the normal function of the body, typically as a result of bacterial, fungal or viral infection in a subject. A disease is a pathological condition of an organ, a body part, or a system resulting from various causes, such as infection, genetic defect (e.g., epidermolysis bullosa (EB) is the name for a group of rare inherited skin disorders that cause the skin to become very fragile. Any trauma or friction to the skin can cause painful blisters), or environmental stress that is characterized by an identifiable group of symptoms. A disorder or disease can refer to a biofilm-related disorder or disorder caused by a planktonic bacterial phenotype that is characterized by a disease-related growth of bacteria. In certain instances, the disease or disorder is a physiological response wherein the immune system causes release of proinflamatory molecules that harm the body. Other diseases and disorders include respiratory diseases, reproductive disorders and skin disorders.

The term “effective amount” or “effective dose” as used herein includes an amount sufficient to achieve the desired result and accordingly will depend on the ingredient and its desired result. Nonetheless, once the desired effect is identified, determining the effective amount is within the skill of a person skilled in the art.

“Halo” or “halogen” as used herein includes fluoro, chloro, bromo, or iodo.

“Heteroatom” as used herein includes an atom other than carbon or hydrogen. Representative heteroatoms include O, S, and N. The nitrogen or sulphur atom of the heteroatom is optionally oxidized to the corresponding N-oxide, S-oxide (sulfoxide), or S-dioxide (sulfone). In a preferred aspect, a heteroatom has at least two bonds to alkylene carbon atoms (e.g., —C₁-C₉ alkylene-O—C₁-C₉ alkylene-). In some embodiments, a heteroatom is further substituted with an acyl, alkyl, aryl, cycloalkyl, heterocyclyl, or heteroaryl group (e.g., —N(Me)-; —N(Ac)—).

“Heteroaroyl” as used herein includes a heteroaryl-C(O)— group wherein heteroaryl is as defined herein. Representative heteroaroyl groups include thiophenoyl, nicotinoyl, pyrrol-2-ylcarbonyl, pyridinoyl, and the like.

“Heterocycloyl” as used herein includes a heterocyclyl-C(O)— group wherein heterocyclyl is as defined herein. Representative heterocycloyl groups include N-methyl prolinoyl, tetrahydrofuranoyl, and the like.

“Hydroxyalkyl” as used herein includes an alkyl group as defined herein substituted with one or more hydroxy groups. Preferred hydroxyalkyls contain lower alkyl. Representative hydroxyalkyl groups include hydroxymethyl and 2-hydroxyethyl.

“Heterocyclyl” as used herein includes an aromatic or non-aromatic monocyclic or multicyclic ring system of about 3 to about 10 ring atoms, preferably about 5 to about 10 ring atoms, in which one or more of the atoms in the ring system is an element or elements other than carbon, e.g., nitrogen, oxygen or sulfur. Preferred heterocyclyl groups contain about 5 to about 6 ring atoms. A heterocyclyl group optionally comprises at least one sp²-hybridized atom (e.g., a ring incorporating a carbonyl, endocyclic olefin, or exocyclic olefin). The prefix “aza,” “oxa,” or “thia” before heterocyclyl means that at least a nitrogen, oxygen or sulfur atom respectively is present as a ring atom. The nitrogen or sulfur atom of the heterocyclyl is optionally oxidized to the corresponding N-oxide, S-oxide or S-dioxide. Representative non-aromatic monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like. Representative aromatic heterocyclyls include pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

A heterocycyl group can be unsubstituted or optionally substituted. When optionally substituted, one or more hydrogen atoms of the group (e.g., from 1 to 4, from 1 to 2, or 1) may be replaced with a moiety independently selected from fluoro, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycyl group is unsubstituted or not optionally substituted.

The term “hydrophobic moiety” or “hydrophobic group” as used herein includes a moiety or a functional group that repels water. Examples may include, but are not limited to, a non-polar alkyl moiety, such as an unsubstituted alkyl group having more than five carbons; a phenyl group; and an anthracenyl group.

As used herein, the terms “hydrophilic moiety” or “hydrophilic group” includes a moiety or a functional group that has a strong affinity to water. Examples may include, but are not limited to, a charged moiety, such as a cationic moiety or an anionic moiety, or a polar uncharged moiety, such as an alkoxy group or an amine group.

As used herein, the term “pharmaceutically acceptable salt” is typically formed from an acid which forms non-toxic acid anions such as the hydrochloride, hydrobromide, sulphate, phosphate or acid phosphate, acetate, maleate, fumarate, lactate, tartrate, citrate, or gluconate salts. Salt formation can be utilized to increase solubility, and therefore, the dissolution rate of a drug. Hydrochloride, mesylate, hydrobromide, acetate, and fumarate are the common counterions that are used for basic chemical entities, while sodium, calcium, and potassium are counterions for weakly acidic drugs.

As used herein, the term “treat,” “treating,” or “treatment” includes administering or applying a composition (e.g., a composition described herein) in an amount, manner (e.g., schedule of administration), and mode (e.g., route of administration) that is effective to improve a disorder or a symptom thereof, or to prevent, to retard, or to slow the progression of a disorder or a symptom thereof. Such improvements can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable.

“A water solubilizing group” is a group that imparts more hydrophilicity to the modified cholic acid component. A water solubilizing group can be an ethylene oxide oligomer. In certain aspects, water solubilizing groups include one or more alkylene oxide repeat units. For example, a water-solubilizing group can contain one or more ethylene glycol units, —(OCH₂CH₂)ₙ—. The PEG group can be any length, however, typically includes between n is 1 to 20 ethylene glycol repeat units. Other PEG derivatives such as CH₃ (OCH₂CH₂)ₙCO or CH₃ (OCH₂CH₂)ₙ wherein n=1 to 15, such as 1, 2, 3, 4, 5, 6, 7, 8, 9, 10, 11, 12, 13, 14, or 15, or n is 4 to 8; or HOOCCH₂CH₂CO—, HOOCCH₂CH₂— or CH₃CO— can be used. One of skill in the art will know of other groups that impart more water solubility to the molecule.

### II. Embodiments

This present disclosure provides compounds, compositions, and methods, wherein the compounds include a modified or cholic acid component and at least a second component in which the second component is conjugated to the modified or cholic acid component. In certain instances, it was surprisingly found that the conjugate compound provides reduced cytotoxicity in use relative to the use of the modified cholic acid component alone. In certain instances, it was surprisingly found that the conjugate compound provides a more robust MOA (e.g., the entry into a cell is more facile or multiple modes of action occur) and increased efficacy in use relative to the use of the modified cholic acid component alone. In certain aspects, the conjugated molecule to the modified cholic acid is a biotin moiety as defined herein, which is appended to a linker defined as L. As used herein, conjugated means a covalent attachment of a cholic acid or a modified cholic acid to a biotin moiety. B¹ or a biotin moiety includes biotin, desthiobiotin as well as biotin mimetics.

Compositions according to the disclosure can be formulated in any therapeutically acceptable manner as described in greater detail below. Further, compositions and methods according to the disclosure provide comparable efficacy in therapeutic treatments relative to treatments incorporating the unconjugated MCA, while nevertheless also providing reduced cytotoxicity.

In certain aspects, the present disclosure provides a cholic acid conjugate, the modified compound having a beneficial effect in the body (such as antimicrobial activity and or immunomodulating activity) but having reduced cell cytotoxicity when the compound is exposed to infected cells or cells for which infection prevention is desired. In certain instances, it was observed that the conjugate compound provides greater efficacy and a more robust MOA (e.g., the entry into a cell is more facile or multiple modes of action occur) when the compound is exposed to infected cells or cells in which infection prevention (e.g., prophylaxis) is desired.

In certain aspects, the modified cholic acid conjugate comprises a modified cholic acid and a second compound, wherein the second compound comprises or is selected from a group of compounds such that when the conjugate so formed is exposed to a desired target a desirable therapeutic outcome is obtained. In certain instances, cytotoxicity at the target site is reduced relative to the degree of cytotoxicity which occurs when the unconjugated form of the modified cholic acid compound is exposed to the same target. In certain instances, the MOA at the target site is more robust relative to the unconjugated form of the modified cholic acid compound when exposed to the same target.

In one embodiment, the present disclosure provides a compound having the following formula I, or a pharmaceutically acceptable salt thereof:

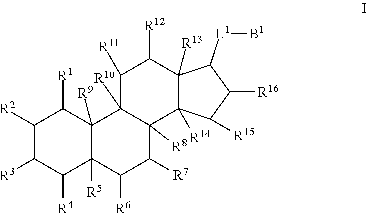

wherein:
R¹-R⁴, R⁶, R⁷, R¹¹, R¹², R¹⁵ and R¹⁶ is each independently a member selected from the group consisting of hydrogen, hydroxyl, a substituted or unsubstituted (C₁-C₁₀) alkyl, a substituted or unsubstituted (C₁-C₁₀) alkylcarboxy, (C₁-C₁₀) hydroxyalkyl, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, a substituted or unsubstituted (C₁-C₁₀) aminoalkylcarboxamido, (C₁-C₁₀) azidoalkyloxy, (C₁-C₁₀) cyanoalkyloxy, (C₁-C₁₀) guanidinoalkyl oxy, and (C₁-C₁₀) guanidinoalkyl carboxy;
R⁵, R⁸, R⁹, R¹⁰, R¹³, and R¹⁴ is each independently a member selected from the group consisting of hydrogen, hydroxyl, a substituted or unsubstituted (C₁-C₁₀) alkyl, (C₁-C₁₀) hydroxyalkyl. (C₁-C₁₀) alkyloxy-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) aminoalkylcarboxy, a substituted or unsubstituted (C₁-C₁₀) aminoalkylaminocarbonyl, (C₁-C₁₀) azidoalkyloxy, (C₁-C₁₀) cyanoalkyloxy, (C₄-C₁₀) guanidinoalkyloxy, and (C₁-C₁₀) guanidinoalkylcarboxy;
L¹ is a linking group; and
B¹ is biotin, desthiobiotin, or biotin mimetics.

In certain aspects, R³, R⁷ and R¹² is each independently a member selected from the group consisting of a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, and a substituted or unsubstituted (C₁-C₁₀) aminoalkylcarboxamido; and R⁵, R⁹ and R¹³ is each independently a member selected from the group consisting of hydrogen, hydroxyl, and a substituted or unsubstituted (C₁-C₁₀) alkyl.

In certain aspects, R³, R⁷ and R¹² is each a substituted or unsubstituted (C₁-C₁₀) amino alkylcarboxy, wherein the amino group is optionally quaternized; and R⁸, R⁹ and R¹³ is each independently a hydrogen or a substituted or unsubstituted (C₁-C₃) alkyl.

In certain aspects, L¹ is -L-Y—Z, wherein L is independently selected from the group consisting of a bond, a substituted or unsubstituted (C₁-C₁₀) alkyl, a substituted or unsubstituted (C₁-C₁₀) alkylcarboxy, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) alkylcarboxamido, a substituted or unsubstituted (C₁-C₁₀) alkylcarboxamido wherein the amino group is substituted, and a substituted or unsubstituted (C₁-C₁₀) alkylaminocarbonyl;

Y is optional and is a member selected from the group consisting of a substituted or unsubstituted (C₁-C₁₀) alkylheterocyclyl (C₁-C₁₀) alkylcarboxamido, a substituted or unsubstituted (C₁-C₁₀) alkylheterocyclyl, a substituted or unsubstituted (C₁-C₁₀) alkylheterocyclyl (C₁-C₁₀) alkylcarboxy, and a substituted or unsubstituted (C₁-C₁₀) alkylheterocyclyl (C₁-C₁₀) alkylaminocarbonyl; and

Z is a member selected from the group consisting of a substituted or unsubstituted C₁-C₃₀ alkylene, a C₁-C₃₀ alkenylene, wherein the alkylene or alkenylene is optionally interrupted by at least one heteroatom, a substituted or unsubstituted (C₁-C₁₀) alkylamino, a substituted or unsubstituted (C₁-C₁₀) alkylcarbonyl, a PEG₁₋₃₀, optionally terminating in a member selected from the group consisting of a bond, —O—, —S—, —NH—, —NHC(O)—, and —C(O)NH.

In certain aspects, B¹ is biotin, desthiobiotin or a biotin mimetic.

In certain aspects, the present disclosure provides a cholic acid conjugate wherein

cholic acid is conjugated to biotin, desthiobiotin or a biotin mimetic.

In another embodiment, the present disclosure provides a method for treating a microorganism infection or retarding the spread of the microorganism, the method comprising:

contacting the microorganism with an antimicrobial amount of a compound having formula I or a pharmaceutically acceptable salt thereof:

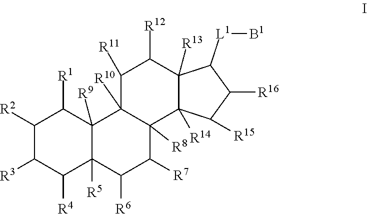

wherein:
R¹-R⁴, R⁶, R⁷, R¹¹, R¹², R¹⁵ and R¹⁶ is each independently a member selected from the group consisting of hydrogen, hydroxyl, a substituted or unsubstituted (C₁-C₁₀) alkyl, a substituted or unsubstituted (C₁-C₁₀) alkyl, a substituted or unsubstituted (C₁-C₁₀) alkylcarboxy, (C₁-C₁₀) hydroxyalkyl, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, a substituted or unsubstituted (C₁-C₁₀) aminoalkylcarboxamido, (C₁-C₁₀) azidoalkyloxy, (C₁-C₁₀) cyanoalkyloxy, (C₁-C₁₀) guanidinoalkyl oxy, and (C₁-C₁₀) guanidinoalkyl carboxy;
R⁵, R⁸, R⁹, R¹⁰, R¹³, and R¹⁴ is each independently a member selected from the group consisting of hydrogen, hydroxyl, a substituted or unsubstituted (C₁-C₁₀) alkyl, (C₁-C₁₀) hydroxyalkyl, (C₁-C₁₀) alkyloxy-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) aminoalkylcarboxy, a substituted or unsubstituted (C₁-C₁₀) aminoalkylaminocarbonyl, (C₁-C₁₀) azidoalkyloxy, (C₁-C₁₀) cyanoalkyloxy, (C₁-C₁₀) guanidinoalkyloxy, and (C₁-C₁₀) guanidinoalkylcarboxy;
L¹ is a linking group; and
B¹ is biotin, desthiobiotin or a biotin mimetic, wherein the compound of formula I kills or slows the spread of the microorganism.

In certain aspects, R³, R⁷ and R¹² is each independently a member selected from the group consisting of a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, and a substituted or unsubstituted (C₁-C₁₀) aminoalkylcarboxamido; and R⁵, R⁹ and R¹³ is each independently a member selected from the group consisting of hydrogen, hydroxyl, and a substituted or unsubstituted (C₁-C₁₀) alkyl.

In certain aspects, R³, R⁷ and R¹² is each a substituted or unsubstituted (C₁-C₁₀) amino alkylcarboxy, wherein the amino group is optionally a cationic amine salt; and R⁵, R⁹ and R¹³ is each independently a hydrogen or a substituted or unsubstituted (C₁-C₃) alkyl.

In certain aspects, L¹ is -L-Y—Z, wherein L is independently selected from the group consisting of a bond, a substituted or unsubstituted (C₁-C₁₀) alkyl, a substituted or unsubstituted (C₁-C₁₀) alkylcarboxy, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) alkylcarboxamido, a substituted or unsubstituted (C₁-C₁₀) alkylcarboxamido wherein the amino group is substituted, and a substituted or unsubstituted (C₁-C₁₀) alkylaminocarbonyl;
Y is optional and is a member selected from the group consisting of a substituted or unsubstituted (C₁-C₁₀) alkylheterocyclyl (C₁-C₁₀) alkylcarboxamido, a substituted or unsubstituted (C₁-C₁₀) alkylheterocyclyl, a substituted or unsubstituted (C₁-C₁₀) alkylheterocyclyl (C₁-C₁₀) alkylcarboxy, and a substituted or unsubstituted (C₁-C₁₀) alkylheterocyclyl (C₁-C₁₀) alkylaminocarbonyl; and
Z is a member selected from the group consisting of a substituted or unsubstituted C₁-C₃₀ alkylene, a C₁-C₃₀ alkenylene, wherein the alkylene or alkenylene is optionally interrupted by at least one heteroatom, a substituted or unsubstituted (C₁-C₁₀) alkylamino, a substituted or unsubstituted (C₁-C₁₀) alkylcarbonyl, a PEG₁₋₃₀, optionally terminating in a member selected from the group consisting of a bond, —O—, —S—, —NH—, —NHC(O)—, and —C(O)NH.

In certain aspects, a biotin moiety includes biotin, desthiobiotin or a biotin mimetic.

In certain aspects, the microorganism is a member selected from the group consisting of a microbe, a bacterium, a virus, and a fungus.

In certain aspects, the microorganism is a virus.

In certain aspects, the virus is a corona virus, such as SARS-COV-2, its variants and subvariants.

In certain aspects, the virus is a Respiratory Syncytial Virus (RSV).

In certain aspects, the virus is an Influenza A virus.

In certain aspects, the present disclosure provides a modified cholic acid conjugated to a second compound, which in certain instances, is a biotin moiety. As used herein, a biotin moiety can be biotin, a desthiobiotin or a biotin memetic.

The chemical structure of cholic acid (6R)-6-[(1R,3aS,3bR,4R,5aS,7R,9aS,9bS,11S,11aR)-4,7,11-trihydroxy-9a,11a-dimethylhexadecahydro-1H-cyclopenta[a]phenanthren-1-yl]heptanoic acid, with CAS Number 81-25-4, is as follows:

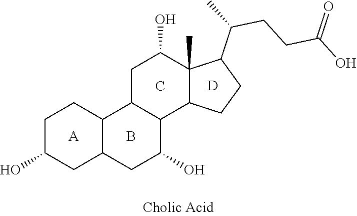

A compound related to or derived from cholic acid is a compound known as a cationic steroidal antimicrobial such as CSA-44. A cationic steroidal antimicrobial is also called a ceragenin. The chemical structure of CSA-44 is as follows:

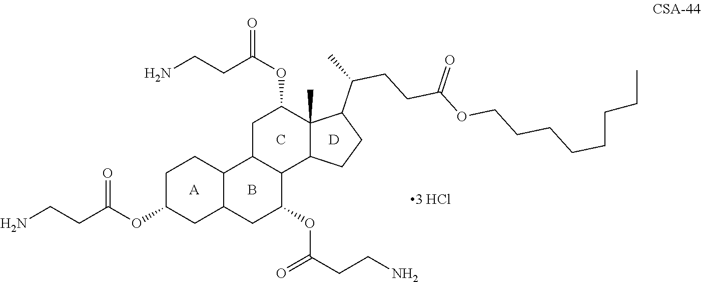

In certain aspects, the modified cholic acid conjugate of the present disclosure has the following structure of Formula Ia:

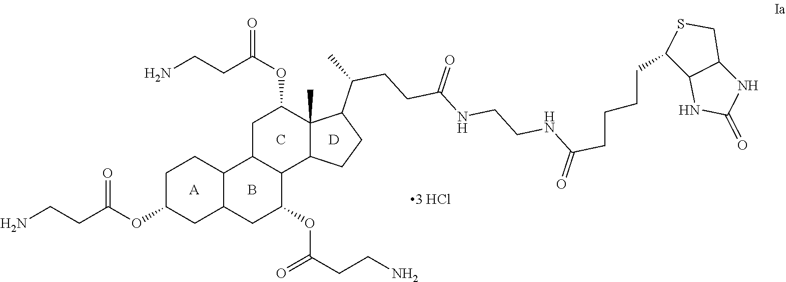

In yet another aspect, a modified cholic acid conjugate is formed from a modified cholic acid and a second compound, where the second compound is a biotin moiety, and the disclosure provides the method of exposing cells in an infected mammal (e.g., infected with a bacteria, virus, fungus or combinations thereof) to the modified cholic acid conjugate, wherein the method of exposing results in cytotoxicity of cells exposed to the conjugate which is less than the degree of cytotoxicity occurring when the cells are exposed to an unconjugated form. In this method of the invention, at least a two-times reduction in cytotoxicity using the conjugate relative to use of the unconjugated ceragenin is attained. In another aspect, the reduction is at least about a four-times reduction (1×, 2×, 3×, 4× or more).

In addition, other scaffolds for modified cholic acids include a chenodeoxycholic acid, (4R)-4-[(1R,3aS,3bR,4R,5aS,7R,9aS,9bS.11aR)-4,7-Dihydroxy-9a,11a-dimethylhexadecahydro-1H-cyclopenta[a]phenanthrene-1-yl]pentanoic acid, with CAS Number 474-25-9, with the following structure:

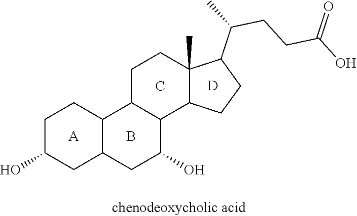

Another scaffold for modified cholic acids includes a deoxycholic acid, (4R)-4-[(1R,3aS,3bR,5aR,7R,9aS,9bS,11S,11aR)-7,11-dihydroxy-9a,11a-dimethylhexadecahydro-1H-cyclopenta[a]phenanthren-1-yl]pentanoic acid, having CAS Number 83-44-3, with the following structure:

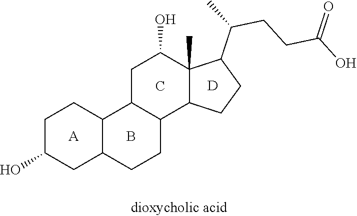

Another scaffold for modified cholic acids includes a lithocholic acid, (4R)-4-[(1R,3aS,3bR,5aR,7R,9aS,9bS,11aR)-7-Hydroxy-9a, 11a-dimethylhexadecahydro-1H-cyclopenta[a]phenanthren-1-yl]pentanoic acid, having a CAS Number 434-13-9, with the following structure:

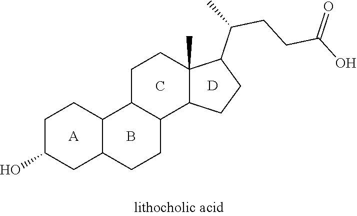

In another aspect, the disclosure comprises, consists of, or consists essentially of a modified colic acid (MCA) conjugate molecule having a therapeutic index of greater than three 3, such as 4, 5, 6, 7, 8, 9, 10, 11, 12, 13, 14, or 15. In another aspect, the disclosure comprises, consists of: or consists essentially of a MCA having a therapeutic index of at least 15, such as 15, 16, 17, 18, 19, 20, 21, 22, 23, 24, 25, or 26. As exemplified herein, toxicity and therapeutic efficacy of such compounds and compositions can be determined by standard pharmaceutical procedures in cell cultures or experimental animals, e.g., for determining the CC₅₀ (the cytotoxic concentration or dose lethal to 50%> of the population) and the EC₅₀ (the concentration or dose therapeutically effective in 50% of the population). The dose ratio between toxic and therapeutic effects is the therapeutic index (“TI”) and it can be expressed as the ratio CC₅₀/EC₅₀.

In certain aspects, the disclosure provides a modified cholic acid conjugated to biotin or a biotin moiety, which conjugate is incorporated into a formulation, and is applied to cells infected with a bacteria, fungi or virus. The modified cholic acid conjugate is present in an amount to provide a favorable antimicrobial (e.g., . . . antibacterial, antiviral, antifungal or a combination thereof) outcome while simultaneously resulting in a level of cytotoxicity less than when such infected cells are exposed to the unconjugated form.

In certain aspects, the disclosure provides a formulation comprising, or consisting essentially of, or consisting of, a modified cholic acid conjugated to biotin or a biotin moiety is administered to a mammal, a human or animal, said mammal having a viral infection, and wherein the degree of infection is reduced and wherein cytotoxicity occurring in the mammal exposed to the conjugate is lower than the degree of cytotoxicity which occurs when an unconjugated form is administered to the mammal to treat such infection. The subject is typically a mammal. The mammal can be a human, non-human primate, mouse, rat, dog, cat, horse, or cow, but are not limited to these examples. Mammals other than humans can be advantageously used, for example, as subjects that represent animal models of, for example, various disorders. In addition, the methods described herein can be used to treat domesticated animals and/or pets. A subject can be male or female.

In certain aspects, the disclosure provides a formulation comprising, or consisting essentially of, or consisting of, a modified cholic acid conjugated to a biotin or biotin moiety is administered to a mammal, particularly a human, said mammal having a viral infection, and wherein the degree of infection is reduced, and cytokine response is favorable compared to untreated infected mammal.

In certain aspects, the disclosure provides methods for treating a disease or condition. In one aspect, disclosure provides treating (or preventing) an infection from a virus such as SARS-COV-2 and variants thereof or Influenza A in a subject such as a human patient comprising the steps of administering a formulation comprising a modified cholic acid conjugated to biotin or a biotin moiety to the patient in an amount effective to treat (or prevent) the infection and wherein the administering results in a degree of cytotoxicity less than the degree of cytotoxicity which results from the administration of an unconjugated form to the patient.

In certain aspects, the disclosure provides treating a corona virus infection from a virus such as SARS-COV-2 and variants thereof or Influenza A in a mammalian subject such as a human patient comprising the steps of administering a formulation comprising a modified cholic acid conjugated to biotin or a biotin moiety to the patient in an amount effective to treat the infection and wherein the treatment results in a degree of cytotoxicity less than the degree of cytotoxicity which results from the administration of an unconjugated form of the modified cholic acid to the patient and wherein the conjugated form incorporates biotin.

In certain instances, the present disclosure provides MCA conjugates having increased efficacy and a more robust mode of action (e.g., the entry into a cell is more facile or multiple modes of action occur) compared to unconjugated MCAs. In addition, the present disclosure provides MCA conjugates having therapeutic indices greater than 3, or alternatively, at least 15 such as 4, 5, 6, 7, 8, 9, 10, 11, 12, 13, 14, 15, 16, 17, 18, 19, 20, 21, 22, 23 24, 25, or even greater.

In certain instances, biotin or a biotin moiety includes biotin, desthiobiotin or a biotin memetic. The biotin memetic can be a short peptide comprising 2-12 amino acids such as 2, 3, 4, 5, 6, 7, 8, 9, 10, 11, or 12 amino acids. The one-letter and three letter designation of amino acids is as follows:

| One-Letter Code | Amino Acid | Three-letter code |
|---|---|---|
| G | Glycine | Gly |
| A | Alanine | Ala |
| L | Leucine | Leu |
| M | Methionine | Met |
| F | Phenylalanine | Phe |
| W | Tryptophan | Trp |
| K | Lysine | Lys |
| Q | Glutamine | Gln |
| E | Glutamic Acid | Glu |
| S | Serine | Ser |
| P | Proline | Pro |
| V | Valine | Val |
| I | Isoleucine | Ile |
| C | Cysteine | Cys |
| Y | Tyrosine | Tyr |
| H | Histidine | His |
| R | Arginine | Arg |
| N | Asparagine | Asn |
| D | Aspartic Acid | Asp |
| T | Threonine | Thr |

### Compounds of the Disclosure

In certain aspects, the disclosure provides a modified cholic acid conjugated to a second compound, wherein the second compound is a vitamin such as biotin. Table 1 provides specific compounds of a modified cholic acid conjugated to biotin:

TABLE 1

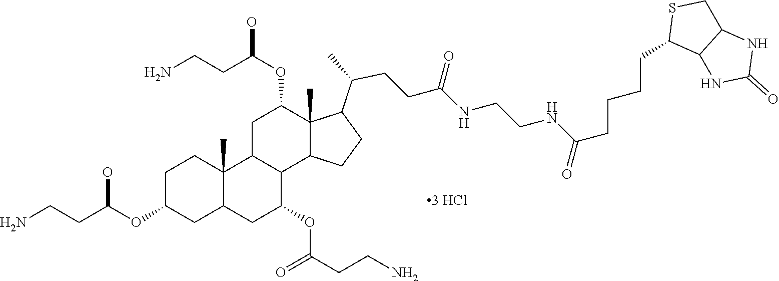 [SMILES: C[C@H](CCC(=O)NCCNC(=O)CCCC[C@@H]1SCC2NC(=O)NC21)C1CCC2C3C(C[C@H](O[C@H](O)CCN)[C@@]21C)[C@@]1(C)CC[C@@H](O[C@H](O)CCN)CC1C[C@H]3OC(=O)CCN]

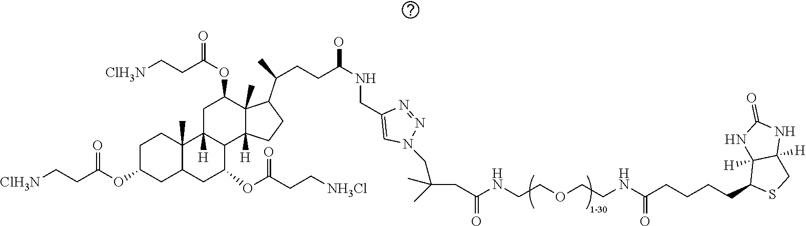

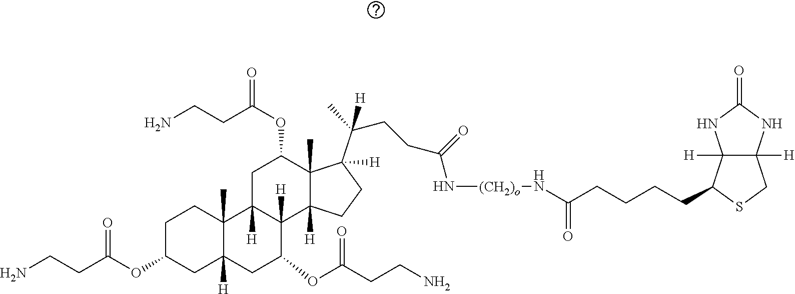

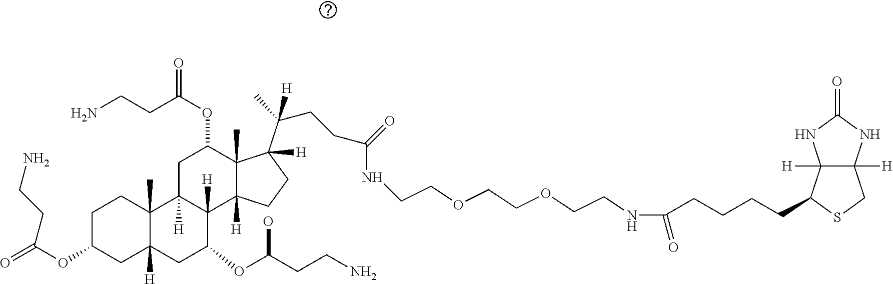

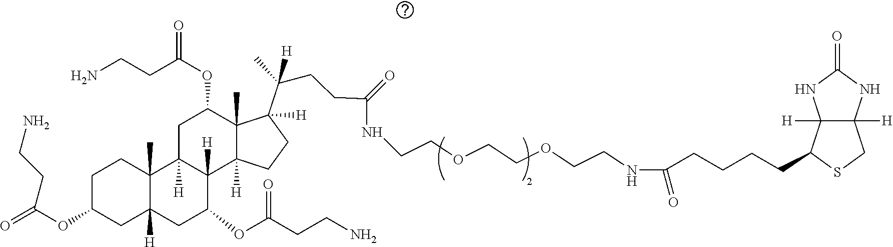

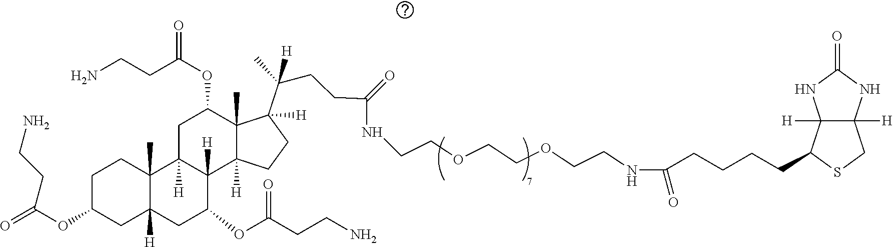

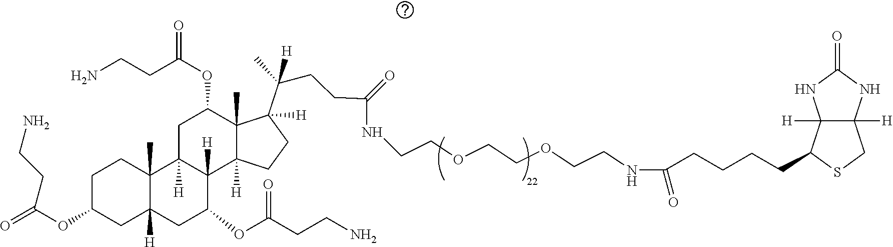

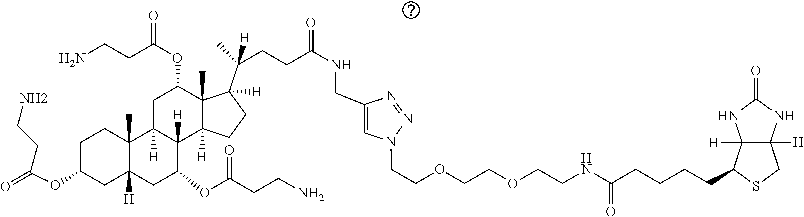

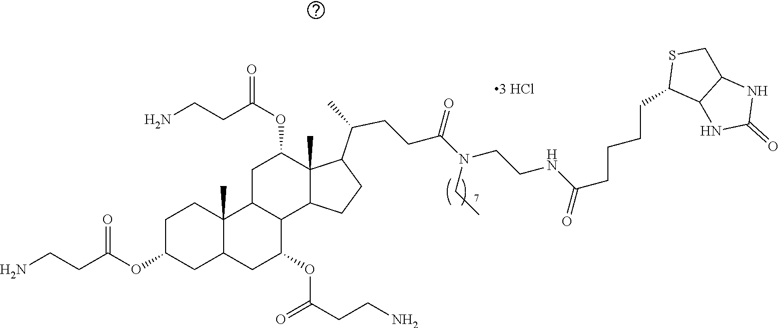

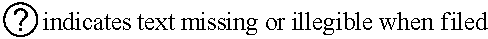

In certain aspects, the disclosure provides a modified cholic acid conjugated to biotin or a biotin moiety comprising a modified cholic acid and a second compound, where the second compound is a desthiobiotin. Table 2 provides specific compounds of a modified cholic acid conjugated to desthiobiotin:

TABLE 2

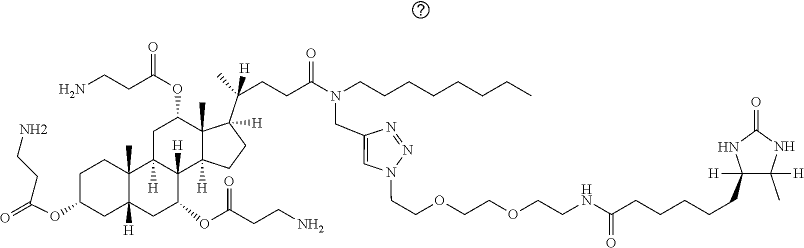

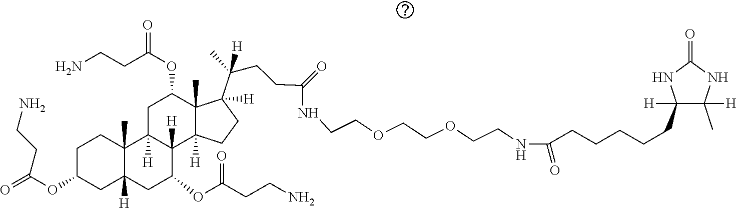

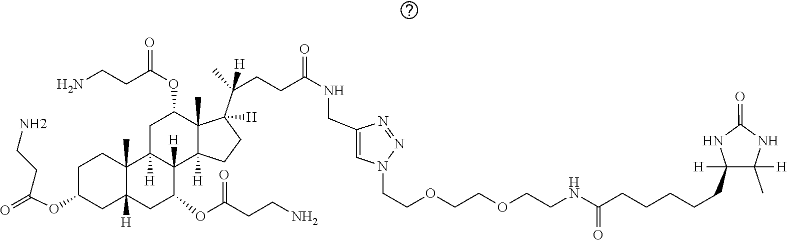

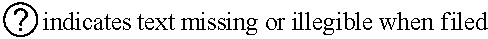

In certain aspects, the disclosure provides a modified cholic acid conjugated to biotin or a biotin moiety comprising a modified cholic acid and a second compound, where the second compound is a biotin memetic. Table 3 provides specific compounds of a modified cholic acid conjugated to a biotin memetic:

TABLE 3

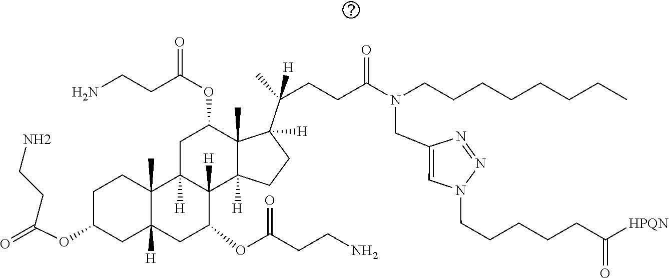

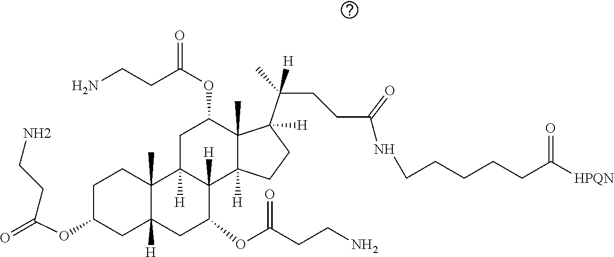

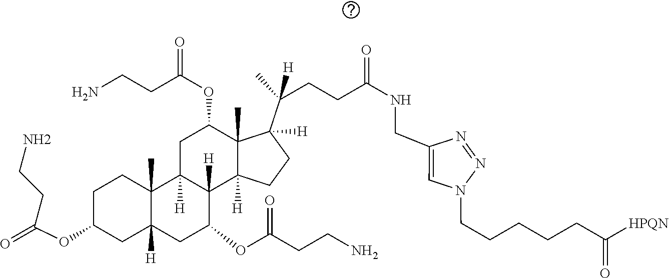

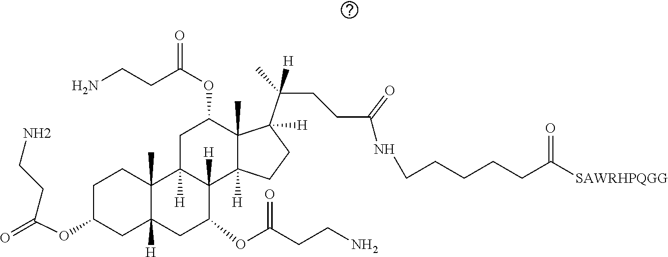

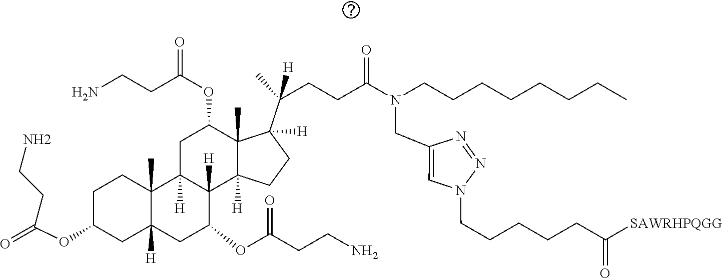

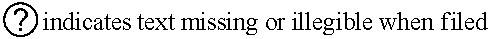

### III. Synthesis

The modified cholic acid can be attached to biotin or a biotin moiety to form a compound of Formula I, which are defined above. “Click” chemistry provides one possible way for linking the modified cholic acid to biotin or a biotin moiety. Click chemistry uses simple, robust reactions, such as the copper-catalyzed cycloaddition of azides and alkynes, to create intermolecular linkages. For a review of click chemistry, see Kolb, H. C.; Finn, M. G.; Sharpless, K. B. Angew. Chem. 2001, 40, 2004.

Connection (or ligation) of two fragments to make a larger molecule or structure is often achieved with the help of so-called “click chemistry” described by Sharpless et al. Angew. Chem, Int. Ed. 40:2004 (2001). This term is used to describe a set of bimolecular reactions between two different reactants such as azides and acetylenes. The formation of 1,2,3-triazoles in 1,3-dipolar cycloaddition of azides to a triple bond is known, but because the activation energy of acetylene-azide cycloaddition is relatively high, the reaction is slow under ambient conditions.

The utility of the reaction of azides with alkynes was expanded by the discovery of Cu (I) catalysis. 1,3-cycloaddition of azides to terminal acetylenes in the presence of catalytic amounts of cuprous salts is facile at room temperature in organic or aqueous solutions.

U.S. Pat. No. 7,807,619 to Bertozzi et al. teaches modified cycloalkyne compounds and method of use of such compounds in modifying a conjugate. Bertozzi et al. teach a cycloaddition reaction that can be carried out under physiological conditions. As disclosed therein, a modified cycloalkyne is reacted with an azide moiety on a target molecule, generating a covalently modified conjugate.

The present disclosure provides compounds of Formula I with click chemistry functionalities useful for labeling biotin or a biotin moiety. As such, in one aspect, the present disclosure provides a modified cholic acid or biotin or a biotin moiety with a member selected from the group consisting of azido, an alkynyl, a pegylated azido group, and a pegylated alkynyl group.

In yet other aspects, the present invention relates to two components that interact with each other to form a stable covalent bio-orthogonal bond. Bio-orthogonal reactions are reactions of materials with each other, wherein each material has limited or essentially no reactivity with functional groups found in vivo. These components are of use in chemical and biological assays, as chemical reagents, medical imaging, and therapy, and more particularly, in nucleic acid modification techniques. According to a particular embodiment of the invention, the covalent bio-orthogonal bond is obtained by the [3+2] cycloaddition of azides and alkynes.

In still other aspects, one of the two components that interact with each other to form a stable covalent bio-orthogonal bond. The starting materials comprise either an azide or an alkyne group on a MCA for use as a reactant in a click chemistry reaction and the other reactant is biotin or a biotin moiety comprising either an alkyne or azide group.

Azide reactive groups such as an alkyne compounds can react with at least one 1,3-dipole-functional compound such as an alkyne reactive group (e.g., a azido group) in a cyclization reaction to form a heterocyclic compound. In certain embodiments, the reaction can be carried out in the presence of an added catalyst (e.g., Cu (I)). In other embodiments, the reaction is carried out in the absence of such catalysts. Exemplary 1,3-dipole-functional compounds include, but are not limited to, azide-functional compounds, nitrile oxide-functional compounds, nitrone-functional compounds, azoxy-functional compounds, and/or acyl diazo-functional compounds. In certain instances, azide-functional compounds are used. Scheme I below is one approach to the Compounds of Formula I:

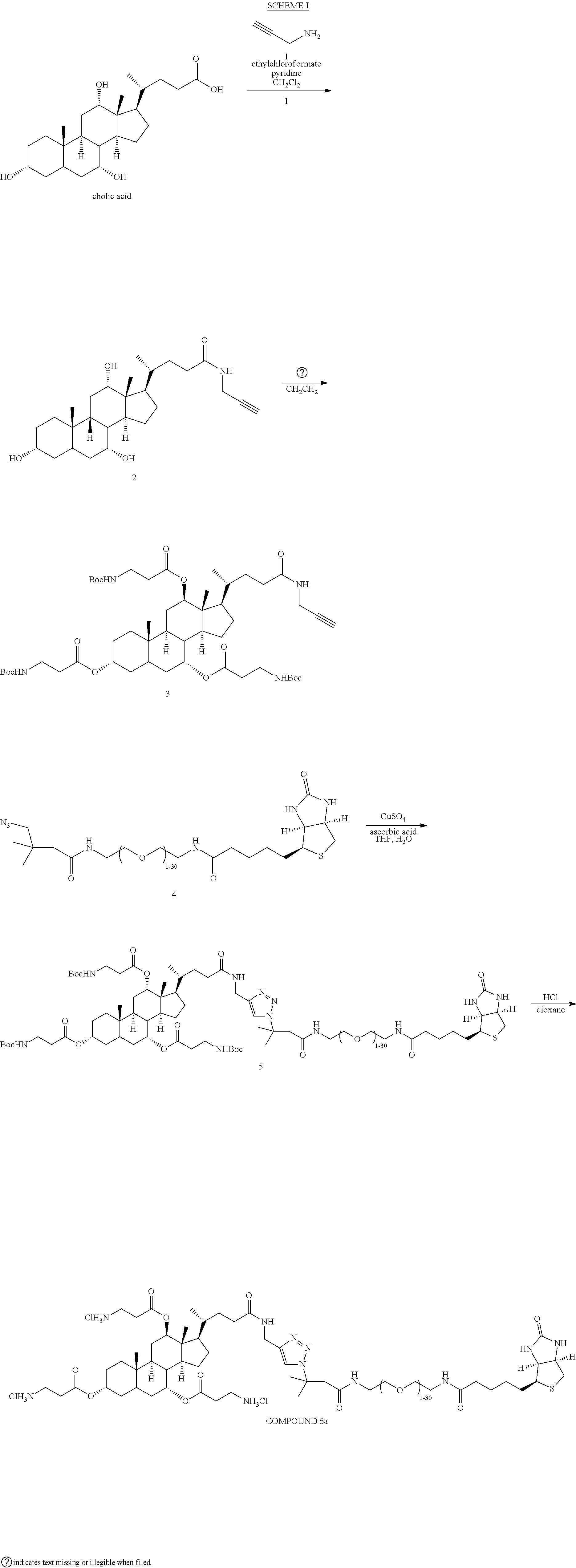

Suitable moieties for a click reaction include, for example, biotin or a biotin moiety.

In one aspect, the compounds of Formula I have sufficient solubility in aqueous solutions that once they are conjugated to a soluble ligand, the ligand retains its solubility. In certain instances, the bioconjugates also have good solubility in organic media (e.g., DMSO or DMF), which provides considerable versatility in synthetic approaches to the labeling of desired materials.

In another aspect, the compounds of Formula I can be prepared as shown in Scheme II below. As shown therein, an active ester of cholic acid 7 is prepared by dropwise addition of benzyl in the presence of camphorsulfonic acid to generate compound 8.

Compound 9 is prepared by the addition of compound 8 to DCC, 4-DMAP, and 3-((tert-butoxycarbonyl) amino) propanoic acid. Upon completion, the crude mixture is dried over anhydrous Na₂SO₄, solvent removed under reduced pressure, and loaded onto Celite. The crude mixture is then purified using SiO₂ chromatography to give Compound 9. Compound 10 is prepared by Pd reduction with hydrogen gas of compound 9 to produce compound 10. Compound 12 is prepared by reacting Compound 10 and Compound 11 (N-(2-aminoethyl)-5-((4S)-2-oxohexahydro-1H-thieno[3,4-d]imidazol-4-yl) pentanamide. The modified cholic acid biotin conjugate is generated.

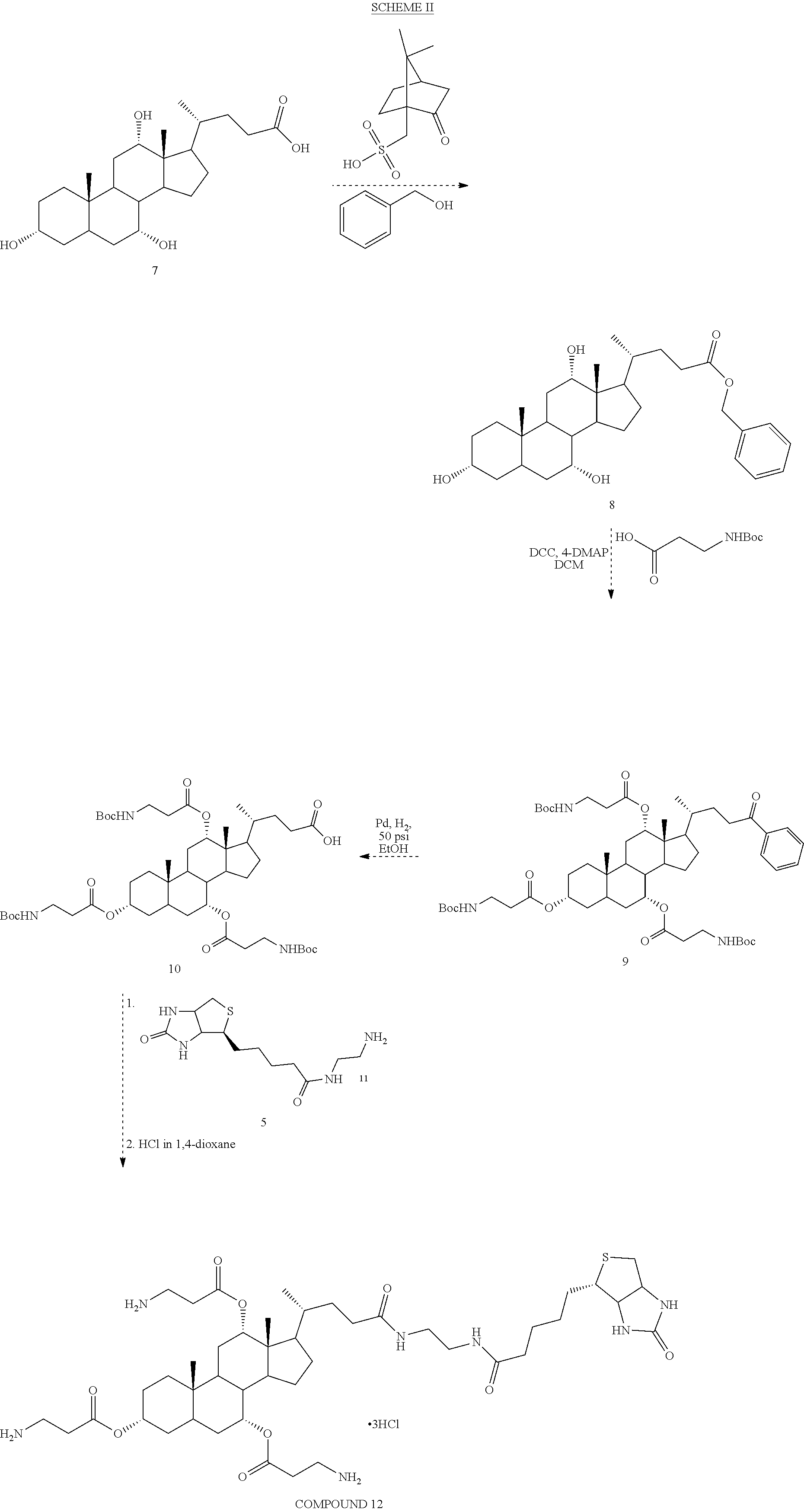

### Peptide-Based MCAs

The disclosure provides biotin memetics such as peptide-based memetics. The disclosed peptides mimic the binding function of biotin with certain short peptides (3-20 amino acids such as 3, 4, 5, 6, 7, 8, 9, 10, 11, 12, 13, 14, 15, 16, 17, 18, 19 or 20 amino acids), particularly peptides containing the HPQN motif within the sequence. Solid Phase Peptide Synthesis (SPPS) techniques have greatly simplified the in vitro synthesis of peptides, including post synthesis labeling and linking group attachment (2). Many peptide manufacturers exist who can make custom peptides to order as a standard service.

In the SPPS process, the peptide is built up one amino acid at a time, each added to the N-terminus of the growing peptide chain. After each amino acid is added, the protecting group on its amine is removed so the next amino acid can be added. When the sequence is completed, the protecting group of the N-terminus is removed. This can either be left as a free amine for attachment to another molecule (e.g. by the activated acid chemistry previously discussed) or a linking group can be added as a spacer and/or to provide a different type of linking chemistry, such as an azide or alkyne for click reactions. When all the desired units have been added, the completed peptide is cleaved from the solid support.

Thus, HPQN tetra peptides with suitable spacer/linker groups on the N-terminus can be readily purchased from vendors specializing in peptide manufacturing. For example, 6-amino caproic acid and 6-azido caproic acid can be used. Many other linkers can be chosen, including direct attachment of the peptide N-terminus to the MCA.

The compounds of the present disclosure are stable at a pH range of about 6 to about 8, such as pH about 6.5 to about 7.5, or at pH values of about 6, about 7, or about 8 such as pH about 7. Storage stability in buffer was monitored over time of at least 7 days to determine the effect of solution pH and degradation. For example, Compound 12 was stable at pH of about 7 with no degradation for a minimum of 7 days. Advantageously, the stability at neutral or near neutral pH of about 7 accelerates wound healing, reduces inflammation, and reduces the risk of cytokine storms during viral infection. In certain aspects, the compounds of Formula I are formulated in a formulation having a pH of about 6 to about 8.

### IV. Applications

The present disclosure provides antimicrobial or antimicrobial conjugates, which kill or slow the spread of microorganisms. Microorganisms or microbes include bacteria, viruses, and fungi such as mold and mildew. The antimicrobial conjugates are not limited to pharmaceuticals, and they may be used topically or in non-therapeutic contexts to control microbial (e.g., bacterial, viral and fungal) growth or biofilms or in non-limiting applications such as anti-inflammatory, pruritis (anti-itching), soothing, urticaria, eczema, pityriasis, psoriasis, erythema, cracked heels, and dermatitis. In addition, the compounds disclosed herein may be used in applications that kill or control microbes on contact. The microbes can also affect a wide variety of biological, medical, and processing operations. Methods and treatments using an antimicrobial conjugate, or a combination of an antimicrobial conjugate with another compound, may include killing, dispersing, treating, reducing, or preventing or inhibiting microbe infection or formation or proliferation.

In some aspects, the formation of a biofilm is inhibited. In other aspects, a previously formed biofilm is dispersed. In still other aspects, substantially all the cells comprising a biofilm are killed.

In some aspects, the described antimicrobial conjugates are suitable in therapeutic and/or prophylactic uses. The latter approach is highly desirable to reduce the probability of viral infection in individuals at high risk, e.g. the elderly (especially those in nursing homes), individuals with co-morbidities, and health care workers, thus substantially reducing the burden on health care systems. An antiviral agent with a high safety profile can be taken pre-exposure or just after contact with an infected individual (post-exposure/therapeutic). The proposed multiple modes-of-action (MOA) will allow pre- and post-treatment of a viral infection.

In some aspects, the described antimicrobial conjugates, alone or in combination with a similar compositions having a sufficient amount of one or more active components, may be provided to a subject as an antimicrobial. The sufficient amount of the one or more antimicrobial conjugates may be sufficient for bacteriostatic, and fungistatic activity. The sufficient amount of the one or more antimicrobial conjugates may be sufficient for bactericidal, virucidal or fungicidal activities. The sufficient amount of the one or more antimicrobial conjugates may be for prophylactic use. The sufficient amount of the one or more antimicrobial conjugates may be for treatment against one or more susceptible pathogens. The sufficient amount of the one or more antimicrobial conjugates may be for treatment against one or more susceptible and antimicrobial-resistant pathogens. Any of the described antimicrobial conjugates either with or without another agent may be provided in one or more suitable, safe, and effective formulations, such as for topical use and/or for internal use. When for internal use, the described antimicrobial conjugate may be formulated for oral ingestion. When for internal use, the described antimicrobial conjugate composition may be formulated for injection. When for internal use, the antimicrobial conjugate may be formulated for inhalation e.g., through a nebulizer, nasal spray or lung inhaler. When for internal use, the antimicrobial conjugate may be formulated for lavage of an organ such as the uterus, bladder, stomach, and intestine.

In some aspects, the described modified cholic acid (MCA) conjugates alone or in combination with a similar composition are useful for therapeutic or prophylactically treatment for viral infections such as enveloped or nonenveloped viruses, or for reducing or preventing spread of lipid envelop virus infections such as SARS-COV-2 and variants thereof, RSV and influenza A viruses.

In some aspects, the described modified cholic acid (MCA) conjugates, alone or in combination with similar compositions, treat bacterial, fungal or viral respiratory infection(s) as well as any secondary infection such as bacterial and/or fungal lung infections in the upper/lower respiratory tracts.

In some aspects, the described modified cholic acid (MCA) conjugates, alone or in combination with a similar composition(s), modulates cytokines and chemokines to reduce cytokine storms, sepsis and other immune-system mediated responses as potential autoimmune disorders.

In some aspects, the described modified cholic acid (MCA) conjugates, alone or in combination with a similar composition are useful for treating respiratory diseases caused by viruses, bacteria, fungi and other pathogens and is not subject to antimicrobial resistance. Treatment is delivered via nebulizers, nasal sprays, lung inhalers and the like. Other delivery systems including oral administration. These applications can be done prophylactically as well as therapeutically.

In certain aspects, the compounds disclosed herein are useful for treating Herpes Zoster, also known as shingles, is a viral infection that causes painful rashes on skin. Herpes Zoster is caused by the Varicella zoster virus, it is the same that causes Chicken pox as well. After the person has had chicken pox, the virus lies dormant in certain nerves for years and its reactivation causes Herpes Zoster Infection. In certain instances, the treatment can be used against a viral infection by applying an ointment/cream, foam and/or spray onto a viral rash or a form such as pimples or blisters.

In certain aspects, the compound disclosed herein are useful for treating skin blisters (herpes rash) caused by herpes simplex virus (HSV-1). Also, another herpes virus (HSV-2) can cause genital herpes and can also pass to an infant during birth. Both forms of the virus enter the nerve cells of the body, where they will remain for life. The virus tends to lie dormant, or asleep, in the cells until something activates it and causes an outbreak of symptoms. Both herpes 1 and 2 can be cured via ointment/cream, spray, foam, or gel.

In some aspects, the microorganism is a member selected from the group consisting of a microbe, a bacterium, a virus, and a fungus.

In some aspects, the microorganism is a virus such as a corona virus (e.g., SARS-CoV-2), RSV, Influenza A, Herpes Virus I and II and Norovirus.

In some aspects, the step of contacting the microorganism is directly or indirectly through meddling in the viral life cycle.

In some aspects, the methods herein provides a beneficial effect in the body as an immunomodulator for regulating toxic immune response to a disease.

Combination with Other Antimicrobials

In certain aspects, this disclosure provides compounds, compositions, or methods, such as industrial, therapeutic, or pharmaceutical compositions, comprising an antimicrobial conjugate in combination with one or more additional active compositions.

In some instances, an antimicrobial conjugate can be administered alone or in combination with a second agent, e.g. a biocide, an antibiotic, or an antimicrobial agent, to thereby kill, disperse, treat, reduce prevent, or inhibit bacteria, viruses or fungi. An antimicrobial such as an antibiotic, antifungal or antiviral can be co-administered with the antimicrobial conjugate either sequentially or simultaneously.

The additional antimicrobial (including antibiotics) can be any compound known to one of ordinary skill in the art that can inhibit the growth of, or kill, bacteria, fungi, viruses, and other forms of pathogens. Useful, non-limiting examples of antibiotics include lincosamides (clindomycin); chloramphenicols; tetracyclines (such as tetracycline, chlortetracycline, demeclocycline, methacycline, doxycycline, minocycline); aminoglycosides (such as gentamicin, tobramycin, netilmicin, smikacin, kanamycin, streptomycin, neomycin); beta-lactams (such as penicillins, cephalosporins, imipenem, aztreonam); glycopeptide antibiotics (such as vancomycin); polypeptide antibiotics (such as bacitracin); macrolides (erythromycins), amphotericins; sulfonamides (such as sulfanilamide, sulfamethoxazole, sulfacetamide, sulfadiazine, sulfisoxazole, sulfacytine, sulfadoxine, mafenide, p-aminobenzoic acid, trimethoprim-sulfamethoxazole); methenamin; nitrofurantoin; phenazopyridine; trimethoprim; rifampicins; metronidazoles; cefazolins; lincomycin; spectinomycin; mupirocina; quinolones (such as nalidixic acid, cinoxacin, norfloxacin, ciprofloxacin, perfloxacin, ofloxacin, enoxacin, fleroxacin, levofloxacin); novobiocins; polymixins; gramicidins; and antipseudomonals (such as carbenicillin, carbenicillin indanyl, ticarcillin, azlocillin, mezlocillin, piperacillin) or any salts or variants thereof. Such antibiotics are commercially available, e.g., from Daiichi Sankyo, Inc. (Parsipanny, NJ), Merck (Whitehouse Station, NJ), Pfizer (New York, NY), Glaxo Smith Kline (Research Triangle Park, NC), Johnson & Johnson (New Brunswick, NJ), AstraZeneca (Wilmington, DE), Novartis (East Hanover, NJ), and Sanofi-Aventis (Bridgewater, NJ). The antibiotic used will depend on the type of bacterial infection.

In certain instances, the compounds of the present disclosure can be combined with one or more of the following such as an antifungal including an allylamine, azole, polyene, pyrimidine, tetraene, thiocarbamate, sulfonamide, a glucan synthesis inhibitor and a benzoic acid compound. Specific antifungals include amrolfine, butenafine, naftifine, terbinafine, ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, undecylenate, silver sulfadiazine, undecylenic acid, undecylenic alkanolamide and Carbol-Fuchsin.

Additional known biocides include biguanide, chlorhexidine, triclosan, chlorine dioxide, and the like. Although one can envision a combination of these biocides and MCA, in other aspects the MCA conjugate can replace one or more of the foregoing.

Useful examples of antimicrobial agents include, but are not limited to, Pyrithiones, especially the zinc complex (ZPT); Octopirox®; dimethyldimethylol hydantoin (Glydant®); methylchloroisothiazolinone/methylisothiazolinone (Kathon CG®); sodium sulfite; sodium bisulfite; imidazolidinyl urea (Germall 115®), diazolidinyl urea (Germaill II®); benzyl alcohol; 2-bromo-2-nitropropane-1,3-diol (Bronopol®); formalin (formaldehyde); iodopropenyl butylcarbamate (Polyphase PI 00®); chloroacetamide; methanamine; methyldibromonitrile glutaronitrile (1,2-dibromo-2,4-dicyanobutane or Tektamer®); glutaraldehyde; 5-bromo-5-nitro-1,3-dioxane (Bronidox®); phenethyl alcohol; o-phenylphenol/sodium o-phenylphenol; sodium hydroxymethylglycinate (Suttocide A®); polymethoxy bicyclic oxazolidine (Nuosept C®); dimethoxane; thimersal; dichlorobenzyl alcohol; captan; chlorphenenesin; dichlorophene; chlorbutanol; glyceryl laurate; halogenated diphenyl ethers; 2,4,4′-trichloro-2′-hydroxy-diphenyl ether (Triclosan®. or TCS); 2,2′-dihydroxy-5,5′-dibromo-diphenyl ether; phenolic compounds; phenol; 2-methylphenol; 3-methylphenol; 4-methylphenol; 4-ethylphenol; 2,4-dimethylphenol; 2,5-dimethylphenol; 3,4-dimethylphenol; 2,6-dimethylphenol; 4-n-propylphenol; 4-n-butylphenol; 4-n-amylphenol; 4-tert-amylphenol; 4-n-hexylphenol; 4-n-heptylphenol; mono- and poly-alkyl and aromatic halophenols; p-chlorophenol; methyl p-chlorophenol; ethyl p-chlorophenol; n-propyl p-chlorophenol; n-butyl p-chlorophenol; n-amyl p-chlorophenol; sec-amyl p-chlorophenol; cyclohexyl p-chlorophenol; n-heptyl p-chlorophenol; n-octyl p-chlorophenol; o-chlorophenol; methyl o-chlorophenol; ethyl o-chlorophenol; n-propyl o-chlorophenol; n-butyl o-chlorophenol; n-amyl o-chlorophenol; tert-amyl o-chlorophenol; n-hexyl o-chlorophenol; n-heptyl o-chlorophenol; o-benzyl p-chlorophenol; o-benxyl-m-methyl p-chlorophenol; o-benzyl-m,m-dimethyl-p-chlorophenol; o-phenylethyl-p-chlorophenol; o-phenylethyl-m-methyl p-chlorophenol; 3-methyl p-chlorophenol; 3,5-dimethyl p-chlorophenol; 6-ethyl-3-methyl p-chlorophenol; 6-n-propyl-3-methyl-p-chlorophenol; 6-isopropyl-3-methyl-p-chlorophenol; 2-ethyl-3,5-dimethyl p-chlorophenol; 6-sec-butyl-3-methyl p-chlorophenol; 2-isopropyl-3,5-dimethyl p-chlorophenol; 6-diethylmethyl-3-methyl p-chlorophenol; 6-isopropyl-2-ethyl-3-methyl p-chlorophenol; 2-sec-amyl-3,5-dimethyl p-chlorophenol; 2-diethylmethyl-3,5-dimethyl p-chlorophenol; 6-sec-octyl-3-methyl p-chlorophenol; p-chloro-m-cresol: p-bromophenol; methyl p-bromophenol; ethyl p-bromophenol; n-propyl p-bromophenol; n-butyl p-bromophenol; n-amyl p-bromophenol; sec-amyl p-bromophenol; n-hexyl p-bromophenol; cyclohexyl p-bromophenol; o-bromophenol; tert-amyl o-bromophenol; n-hexyl o-bromophenol; n-propyl-m,m-dimethyl-o-bromophenol; 2-phenylphenol; 4-chloro-2-methylphenol; 4-chloro-3-methyl phenol; 4-chloro-3,5-dimethyl phenol; 2,4-dichloro-3,5-dimethylphenol; 3,4,5,6-tetrabromo-2-methyl-phenol; 5-methyl-2-pentylphenol; 4-isopropyl-3-methylphenol; p-chloro-m-xylenol (PCMX); chlorothymol; phenoxyethanol; phenoxyisopropanol; 5-chloro-2-hydroxydiphenylmethane; resorcinol and its derivatives; resorcinol; methyl resorcinol; ethyl resorcinol; n-propyl resorcinol; n-butyl resorcinol; n-amyl resorcinol; n-hexyl resorcinol; n-heptyl resorcinol; n-octyl resorcinol; n-nonyl resorcinol; phenyl resorcinol; benzyl resorcinol; phenylethyl resorcinol; phenylpropyl resorcinol; p-chlorobenzyl resorcinol; 5-chloro 2,4-dihydroxydiphenyl methane; 4′-chloro 2,4-dihydroxydiphenyl methane; 5-bromo 2,4-dihydroxydiphenyl methane; 4′-bromo 2,4-dihydroxydiphenyl methane; bisphenolic compounds; 2,2′-methylene bis-(4-chlorophenol); 2,2′-methylene bis-(3,4,6-trichlorophenol); 2,2′-methylene bis(4-chloro-6-bromophenol); bis(2-hydroxy-3,5-dichlorophenyl) sulfide; bis(2-hydroxy-5-chlorobenzyl) sulfide; benzoic esters (parabens); methylparaben; propylparaben; butylparaben; ethylparaben; isopropylparaben; isobutylparaben; benzylparaben; sodium methylparaben; sodium propylparaben; halogenated carbanilides; 3,4,4′-trichlorocarbanilides (e.g., Triclocarban® or TCC); 3-trifluoromethyl-4,4′-dichlorocarbanilide; 3,3′,4-trichlorocarbanilide; chlorohexidine and its digluconate; diacetate and dihydrochloride; undecenoic acid; thiabendazole, hexetidine; and poly(hexamethylenebiguanide) hydrochloride (Cosmocil®).

In certain instances, a compound of the disclosure is combined with an antiviral drug. Antiviral agents function as either viral targets or host factors. Virus-targeting antivirals function through a direct or an indirect method in the viral life cycle. Host-targeting antivirals include reagents that target the host proteins that are involved in the viral life cycle. Direct virus-targeting antiviral drugs include attachment inhibitors, entry inhibitors, fusion inhibitors, uncoating inhibitors, protease inhibitors, polymerase inhibitors, nucleoside and nucleotide reverse transcriptase inhibitors, nonnucleoside reverse-transcriptase inhibitors, and integrase inhibitors. The additional antiviral drug can be any compound known to one of ordinary skill in the art that can kill the virus directly, prevent its entry into a cell, or retards or stops its replication inside the cells. In certain instances, the antiviral is a nucleoside analog, b) a non-nucleoside polymerase inhibitor, or a protease inhibitor. In certain instances, host-targeting antivirals include Cyclophilin inhibitors (Alisporivir), HIV-1 co-receptor antagonists (Aplaviroc, Vicriviroc, etc.). Indirect virus-targeting antivirals include those interaction blockers during the viral life cycle. In certain instances, indiret virus-taregting antivirals include RTC blockers (BMS790052), and RNP blockers (Nucleozin). Useful, nonlimiting examples of antiviral drugs include Acylovir, Remdesivir, Oseltamivir, Maraviroc, Ribavirin, Tromantadine, Nitazoxanide, Lopinavir, Ensitrelvir, and Adefovir.

In certain instances, the compounds of the present disclosure can be combined with an antiviral such as protease inhibitors (PIs), nucleoside reverse transcriptase inhibitors (NRTIs), nucleotide reverse transcriptase inhibitors (NtRTIs), non-nucleoside reverse transcriptase inhibitors (NNRTIs), integrase inhibitors, entry inhibitors, maturation inhibitors and pharmaceutically-acceptable salts and precursors thereof. Specific examples include, aciclovir, docosanol, edoxudine, famciclovir, foscarnet, idoxuridine, penciclovir, trifluridine, tromantidine, valaciclovir and vidarabine (all of which treat infection caused by one or more herpes viruses); adefovir, boceprevir, entecavir, ribavirin and taribavirin (all of which treat infection caused by one or more hepatitis viruses); amantadine, arbidol, oseltamivir, peramivir, rimantidine and zanamivir (all of which treat infection cause by one or more influenza viruses). Other antiviral include amprenavir, atazanavir, darunavir, fosamprenavir, indinavir, lopinavir, nelfinavir, ritonavir, saquinavir and tipranavir (all of which are protease inhibitors); abacavir (ABC), amdoxovir, apricitabine (ATC), didanosine (ddl), elvucitabine, emtricitabine (FTC), entecavir (INN), lamivudine (3TC), racivir, stampidine, stavudine (d4T), zalcitabine (ddC) and zidovudine (AZT) (all of which are NRTIs); adefovir (also known as bis-POM PMPA) and tenofovir (both of which are NtRTIs); delavirdine, efavirenz, etravirine, lersivirine, loviride, nevirapine and rilpivirine (all of which are NNRTIs); elvitegravir, globoidnan A, GSK-572, MK-2048 and raltegravir (all of which are integrase inhibitors); enfuviritide, ibalizumab, maraviroc and vicriviroc (all of which are fusion/entry inhibitors); bevirimat and vivecon (both of which are maturation inhibitors); and pharmaceutically-acceptable salts and precursors thereof, and mixtures thereof. The compounds disclosed herein exhibit protease inhibitory activities, and thus, the current disclosure provides combination therapy with a compound of Formula I and RNA-Dependant-RNA Polymerase (RdRp) inhibitor such as Molnupiravir.

### V. Formulations

The modified cholic acid conjugate antimicrobials provided herein can be incorporated into pharmaceutical compositions. The antimicrobial conjugate, or combination of an antimicrobial conjugate with another compound, can be incorporated into pharmaceutical compositions as pharmaceutically acceptable salts or derivatives. Some pharmaceutically acceptable derivatives of the antimicrobial conjugates of the present invention may include a chemical group, which increases aqueous solubility. As used herein, a “pharmaceutically acceptable carrier” means a carrier that can be administered to a subject together with an antimicrobial conjugate or combination of an antimicrobial conjugate with another compound, described herein, which does not destroy the pharmacological activity thereof. Pharmaceutically acceptable carriers include, for example, solvents, binders, dispersion media, coatings, preservatives, colorants, isotonic and absorption delaying agents, and the like, compatible with pharmaceutical administration. Supplementary active compounds can also be incorporated into the compositions.

Non-limiting examples of pharmaceutically acceptable carriers that can be used include poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid (PLGA), polycaprolactone, polyvalerolactone, poly(anhydrides), copolymers of polycaprolactone with polyethylene glycol, copolymers of polylactic acid with polyethylene glycol, polyethylene glycol; and combinations and blends thereof.

Other carriers include, e.g., an aqueous gelatin, an aqueous protein, a polymeric carrier, a cross-linking agent, or a combination thereof. In other instances, the carrier is a matrix. In yet another instances, the carrier includes water, a pharmaceutically acceptable buffer salt, a pharmaceutically acceptable buffer solution, a pharmaceutically acceptable antioxidant, ascorbic acid, one or more low molecular weight pharmaceutically acceptable polypeptides, a peptide comprising about 2 to about 10 amino acid residues, one or more pharmaceutically acceptable proteins, one or more pharmaceutically acceptable amino acids, an essential-to-human amino acid, one or more pharmaceutically acceptable carbohydrates, one or more pharmaceutically acceptable carbohydrate-derived materials, a non-reducing sugar, glucose, sucrose, sorbitol, trehalose, mannitol, maltodextrin, dextrins, cyclodextrin, a pharmaceutically acceptable chelating agent, EDTA, a chelating agent for a divalent metal ion, a chelating agent for a trivalent metal ion, glutathione, pharmaceutically acceptable nonspecific serum albumin, or combinations thereof.

In other embodiments, the compositions can also comprise a pharmaceutically acceptable carrier. In still other embodiments the effective amount is an amount effective to treat or prevent a disease or disorder. In some embodiments, an effective amount comprises and amount effective to treat or prevent a biofilm.

In some embodiments, the compositions discussed herein further comprises an agent suitable for application to the surface. In other embodiments, the composition is formulated as a wash solution, a dressing, a wound gel, or a synthetic tissue. In further embodiments, the composition is formulated as tablets, pills, troches, capsules, liquid spray, aerosol spray, solutions, suspensions, gels, pastes, lotions, ointments, creams, or foams. In some embodiments, the composition is formulated for parenteral (e.g., intravenous), intradermal, subcutaneous, oral (e.g., inhalation), transdermal (topical), transmucosal, vaginal, or rectal administration.

In some embodiments, the antimicrobial conjugate or combination of an antimicrobial conjugate and at least one other composition is formulated as a slow-release formulation.

In some embodiments, the antimicrobial conjugate or combination of an antimicrobial conjugate and at least one other composition are directed for use in medical applications, for example, active release or passive antimicrobial coatings for lavage solutions for open wounds, toothpaste additives, hand sanitizers, systemic prophylactic antimicrobials, lock solutions for catheters, eye drop solutions for irrigation and contact lens cleaners, prophylactic dental inserts, high level disinfectants, gastrointestinal (GI) tract oral medications for the treatment of infections such as those caused by Shigella, Cryptosporidium, Vibrio cholerae, or Clostridium difficile, topical ointments to treat dermatological complications including infection, canker sores, psoriasis, herpes, chronic wounds, diaper rash, onychomycosis (athletes foot), tinea unguium (toenail fungus), ulcers, or acne, etc.

In some embodiments, the compounds can be used for skin-care preparations, cosmetic personal care preparations, intimate hygiene preparations, and foot care preparations.

A pharmaceutical composition containing an antimicrobial conjugate, or combination of a an antimicrobial conjugate with another compound, can be formulated to be compatible with its intended route of administration as known by those of ordinary skill in the art. Nonlimiting examples of routes of administration include parenteral, e.g., intravenous, intradermal, subcutaneous, oral (e.g., inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition can be sterile and can be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be desirable to include isotonic agents, for example, sugars, polyalcohols such as mannitol, sorbitol, or sodium chloride in the composition. Prolonged absorption of the injectable compositions can be accomplished by including in the composition an agent that delays absorption, for example, aluminum monostearate and gelatin (see, e.g., Remington: The Science and Practice of Pharmacy, 21^{st} edition, Lippincott Williams & Wilkins, Gennaro, ed. (2006)).

Sterile injectable solutions can be prepared by incorporating an antimicrobial conjugate, or combination of an antimicrobial conjugate with another compound, in the required amount in an appropriate solvent with one or a combination of ingredients enumerated above, as required, followed by filtered sterilization. Generally, dispersions are prepared by incorporating an active compound into a sterile vehicle that contains a basic dispersion medium and the required other ingredients from those enumerated above. In the case of sterile powders for the preparation of sterile injectable solutions, the methods of preparation include, without limitation, vacuum drying and freeze-drying which yields a powder of the active ingredient plus any additional desired ingredient from a previously sterile-filtered solution thereof.

Oral compositions may include an inert diluent or an edible carrier or binders. For the purpose of oral therapeutic administration, an antimicrobial conjugate, or a combination of antimicrobial conjugates, or combination of an antimicrobial conjugate with another compound, can be incorporated with excipients and used in the form of tablets, chewing gums, lollipops, pills, troches, chews or capsules, e.g., gelatin capsules. Also included are delivery mechanisms to the stomach and gastrointestinal tract. Pharmaceutically compatible binding agents, or adjuvant materials can be included as part of the composition. The tablets, pills, capsules, troches, chews and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, an antimicrobial conjugate, or combination of a an antimicrobial conjugate with another compound, can be delivered by a nebulizer, nasal spray, lung inhaler or in the form of an aerosol spray from pressured container or dispenser that contains a suitable propellant, e.g., a gas such as carbon dioxide, a nasal spray, a lung inhaler or an in-patient or out-patient nebulizer.

Systemic administration can also be by transmucosal or transdermal means. For transmucosal or transdermal administration, penetrants appropriate to the barrier to be permeated are used in the formulation. Such penetrants are generally known in the art, and include, but are not limited to, for example, for transmucosal administration, detergents, bile salts, and fusidic acid derivatives. Transmucosal administration can be accomplished through the use of nasal sprays, lung inhaler, nebulizers or suppositories. For transdermal administration, the active compounds and compositions are formulated into pharmaceutically acceptable formulation embodiments, such as ointments, salves, gels, skin washes/cleansers or creams as generally known in the art.

For treatment of acute or chronic wounds, an antimicrobial conjugate, or combination of an antimicrobial conjugate with another compound, can be formulated as a dressing, a wash solution, gel, salve, foams ointment or a synthetic tissue, etc.

The pharmaceutical compositions containing an antimicrobial conjugate, or combination of an antimicrobial conjugate with another compound, can also be prepared in the form of suppositories (e.g., with conventional suppository bases such as cocoa butter and other glycerides) or retention enemas for rectal delivery.

Some pharmaceutical compositions containing an antimicrobial conjugate, or combination of an antimicrobial conjugate with another compound, can be prepared with a carrier that protects the antimicrobial conjugate, or combination of an antimicrobial conjugate with another compound, against conjugate degradation, rapid elimination from the body, such as a controlled release formulation, including implants and microencapsulated delivery systems as described, e.g., in Tan et al., Pharm. Res. 24:2297-2308 (2007).

Additionally, biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art.

The materials can also be obtained commercially. Liposomal suspensions (including liposomes targeted to particular cells with monoclonal antibodies to cell surface antigens) can also be used as pharmaceutically acceptable carriers. These can be prepared according to methods known to those skilled in the art, e.g., as described in U.S. Pat. No. 4,522,811.

Toxicity and therapeutic efficacy of such compounds and compositions can be determined by standard pharmaceutical procedures in cell cultures or experimental animals, e.g., for determining the CC₅₀ (the cytotoxic concentration or dose lethal to 50%> of the population) and the EC₅₀ (the concentration or dose therapeutically effective in 50% of the population). The dose ratio between toxic and therapeutic effects is the therapeutic index (“TI”) and it can be expressed as the ratio CC₅₀/EC₅₀. While compounds and compositions that exhibit toxic side effects can be used, care should be taken to design a delivery system that targets active components to the site of affected tissue in order to minimize potential damage to normal cells and, thereby, reduce side effects.

The data obtained from the cell culture assays and animal studies can be used in formulating a range of dosage for use in humans. The dosage of such compounds and compositions lies generally within a range of circulating concentrations that include the ED₅₀ with little or no toxicity. The dosage can vary within this range depending upon the dosage form employed and the route of administration utilized. For any compounds or compositions used in the methods described herein, the therapeutically effective dose can be estimated initially from cell culture assays. A dose can be formulated in animal models to achieve a circulating plasma concentration range that includes the IC₅₀ (i.e., the concentration of the test compound or composition that achieves a half-maximal inhibition of symptoms) as determined in cell culture. Such information can be used to more accurately determine useful doses in humans. Levels in plasma can be measured, for example, by high performance liquid chromatography. Information for preparing and testing such compositions are known in the art. See, e.g., Remington: The Science and Practice of Pharmacy, 21^{st} ed., Lippincott Williams & Wilkins, Gennaro, ed. (2006).

A physician will appreciate that certain factors may influence the dosage required to effectively treat a subject, including but not limited to the severity of the disease or disorder, previous treatments, the general health or age of the subject, and other diseases present. Moreover, treatment of a subject with a therapeutically effective amount of an antimicrobial conjugate, or combination of an antimicrobial conjugate with another compound, can include a single treatment or a series of treatments.

The compounds or pharmaceutical compositions can be included in a container, pack, or dispenser together with instructions for administration. A person of ordinary skill in the art will appreciate that the compounds or pharmaceutical compositions described herein can be formulated as single-dose vials, or single or multi-dose packets for nasal sprays, nebulizers lung inhalers or other dispensing devices.

Antimicrobial conjugates or combination of an antimicrobial conjugate with another compound, may be suitable as antibiofilm active substances in personal care preparations, for example shampoos, bath additives, hair care preparations, liquid and solid soaps (based on synthetic surfactants and salts of saturated or unsaturated fatty acids), lotions and creams, deodorants, other aqueous or alcoholic solutions, e.g. cleansing solutions for the skin, moist cleaning cloths, oils or powders.

### VI. Examples

Example 1 is a description of the synthesis of a cholic acid biotin conjugate (Compound 12).

To a round bottom flask containing a stir bar and solvent is added Cholic acid. While stirring at room temperature, camphorsulfonic acid is then added followed by the dropwise addition of benzyl alcohol. The reaction is allowed to continue stirring at room temperature, monitoring via TLC. Upon completion, the crude mixture is dried over anhydrous Na₂SO₄, solvent removed under reduced pressure, and loaded onto Celite. The crude mixture is then purified using SiO₂ chromatography to give Compound 8.

Compound 9: To a round bottom flask containing a stir bar, DCM is added and cooled to 0° C. in an ice bath. DCC, 4-DMAP, and 3-((tert-butoxycarbonyl) amino) propanoic acid are added and allowed to stir for 5 minutes while maintaining temperature. Compound 8 is dissolved in minimal DCM and added to the reaction mixture while stirring. The reaction is allowed to warm to room temperature and stir for an additional 3 h, monitoring via TLC. Upon completion, the crude mixture is dried over anhydrous Na₂SO₄, solvent removed under reduced pressure, and loaded onto Celite. The crude mixture is then purified using SiO₂ chromatography to give Compound 9.

Compound 10: A two neck round bottom flask containing a stir bar and fitted with septa is flame dried, purging and backfilling with N₂ 3×. EtOH is then added, followed by Compound 9 and again purged, backfilling with N₂. Pd is added, and the reaction is allowed to stir at room temperature under positive pressure of H₂ for 3 h. Upon completion, the reaction mixture is filtered through Celite washing with EtOAc 3×. The combined crude mixture is dried over anhydrous Na₂SO₄, solvent removed under reduced pressure, and loaded onto Celite. The crude mixture is purified using SiO₂ chromatography to give Compound 10.

Compound 12: To a round bottom flask containing a stir bar is added dry THF, Compound 10, and N-(2-aminoethyl)-5-((4S)-2-oxohexahydro-1H-thieno[3,4-d]46midazole-4-yl) pentanamide (Compound 11). The reaction mixture is allowed to stir, monitoring via TLC. 4 M HCl in 1,4-dioxane is then added and reaction mixture is allowed to continue stirring. Upon completion, the crude mixture is dried over anhydrous Na₂SO₄, solvent removed under reduced pressure, and loaded onto Celite. The crude mixture is purified using SiO₂ chromatography to give the cholic acid biotin conjugate Compound 12 as a white solid. ¹H NMR (CD₃OD, 500 MHZ); HRMS ESI+ [M+H]⁺ m/z 890.

¹H NMR and ¹³C NMR spectra were recorded and are referenced to TMS, residual CHD₂OD (¹H) or CD₃OD (¹³C). Mass spectrometric data were obtained. Compound purity was obtained via Ultra-Performance Liquid Chromatography. Reagents and solvents were obtained commercially and used as received.

Example 2 is a contemplated preparation of (3R,7R,9S,10S,12S,13R,14S,17S)-17-(4-(((1-(1-((3aS,4S,6aR)-3a,6a-dimethyl-2-oxohexahydro-1H-thieno[3,4-d]imidazol-4-yl)-43,43-dimethyl-3,41-dioxo-7,10,13,16,19,22,25,28,31,34,37-undecaoxa-4,40-diazatetratetracontan-44-yl)-1H-1,2,3-triazol-4-yl)methyl)amino)-4-oxobutyl)-10,13-dimethylhexadecahydro-1H-cyclopenta[a]phenanthrene-3,7,12-triyl tris(3-(chloro-15-azaneyl) propanoate) (6a). In Scheme 1 propargyl amine can be used as a starting reagent to yield compound 6a.

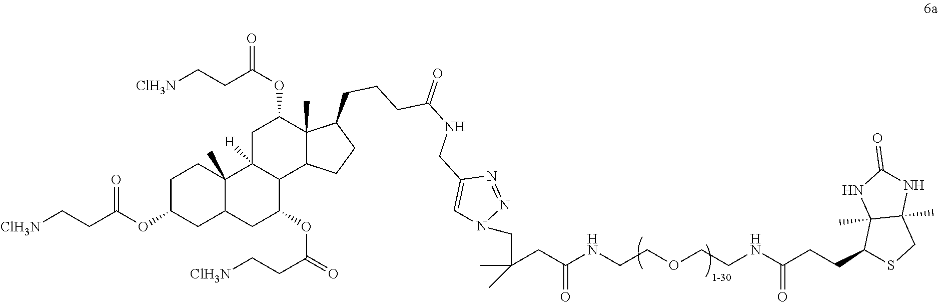

Example 3 is a contemplated preparation of (3R,7R,10S,12S,13R)-10,13-dimethyl-17-((2R)-5-oxo-5-((6-(5-((4R)-2-oxohexahydro-1H-thieno[3,4-d]imidazol-4-yl) pentanamido) hexyl)amino) pentan-2-yl) hexadecahydro-1H-cyclopenta[a]phenanthrene-3,7,12-triyl tris(3-aminopropanoate) (12a). Compound 11 from Scheme 2 may be replaced with compound 11a with the same conditions to produce longer alkyl chain linkages between the biotin and the remaining amine to generate compounds 12a.

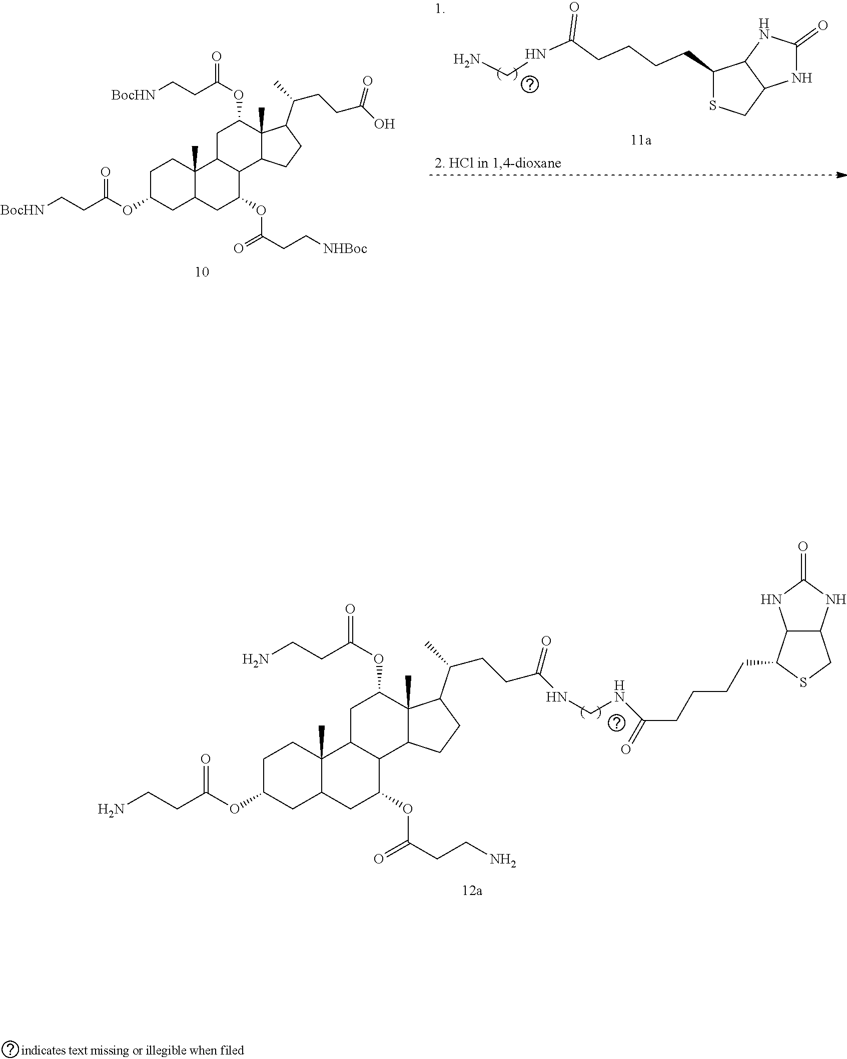

Example 3A is a contemplated preparation of (3R,7R,10S,12S,13R)-10,13-dimethyl-17-((2R)-5-(octyl(2-(5-((4R)-2-oxohexahydro-1H-thieno[3,4-d]imidazol-4-yl) pentanamido)ethyl)amino)-5-oxopentan-2-yl) hexadecahydro-1H-cyclopenta[a]phenanthrene-3,7,12-triyl tris(3-aminopropanoate) (12a′). Compound 11 from Scheme 2 may be replaced with compound 11a′ with the same conditions to produce longer alkyl chain linkages between the biotin and the remaining amine to generate compounds 12a′.

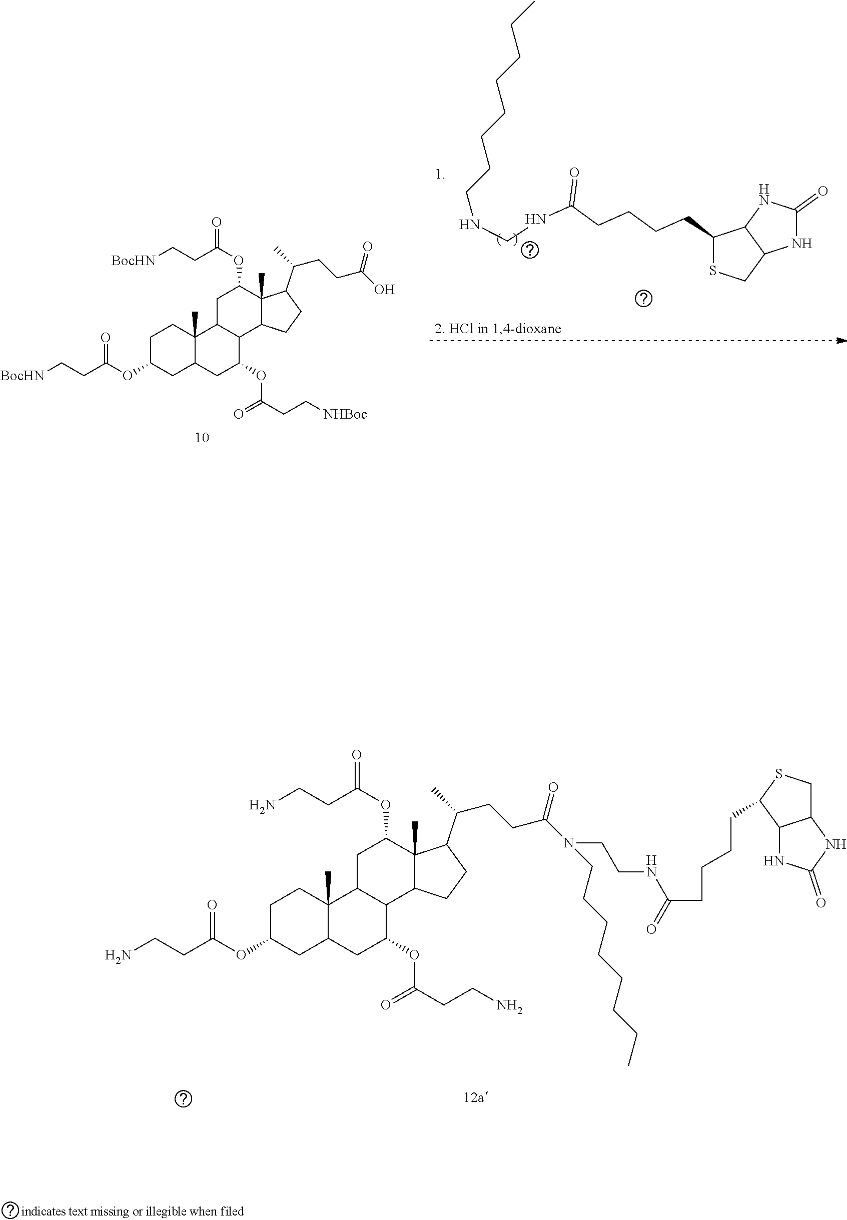

Example 4 is a contemplated preparation of (3R,7R,10S,12S,13R)-17-((2R)-5,16-dioxo-20-((4R)-2-oxohexahydro-1H-thieno[3,4-d]imidazol-4-yl)-9,12-dioxa-6,15-diazaicosan-2-yl)-10,13-dimethylhexadecahydro-1H-cyclopenta[a]phenanthrene-3,7,12-triyl tris(3-aminopropanoate) (12b). Compound 11 from Scheme 2 may be replaced with compound 11b with the same conditions to produce longer alkyl chain linkages between the biotin and the remaining amine to generate compound 12b.

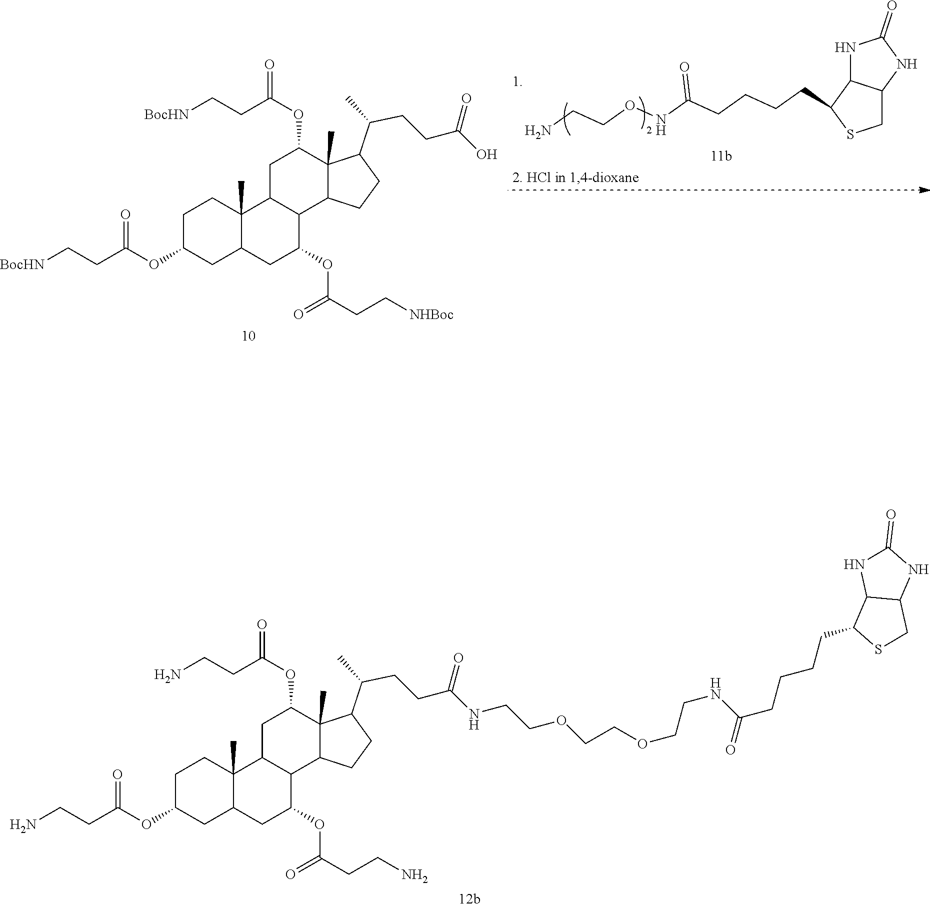

Example 5 is a contemplated preparation of (3R,7R,10S,12S,13R)-17-((27R)-5,24-dioxo-1-((4R)-2-oxohexahydro-1H-thieno[3,4-d]imidazol-4-yl)-9,12,16,20-tetraoxa-6,23-diazaoctacosan-27-yl)-10,13-dimethylhexadecahydro-1H-cyclopenta[a]phenanthrene-3,7,12-triyl tris(3-aminopropanoate) (12c). Compound 11 from Scheme 2 may be replaced with compound 11c with the same conditions to produce a longer chain linkages between the biotin and the remaining amine to generate compound 12c.

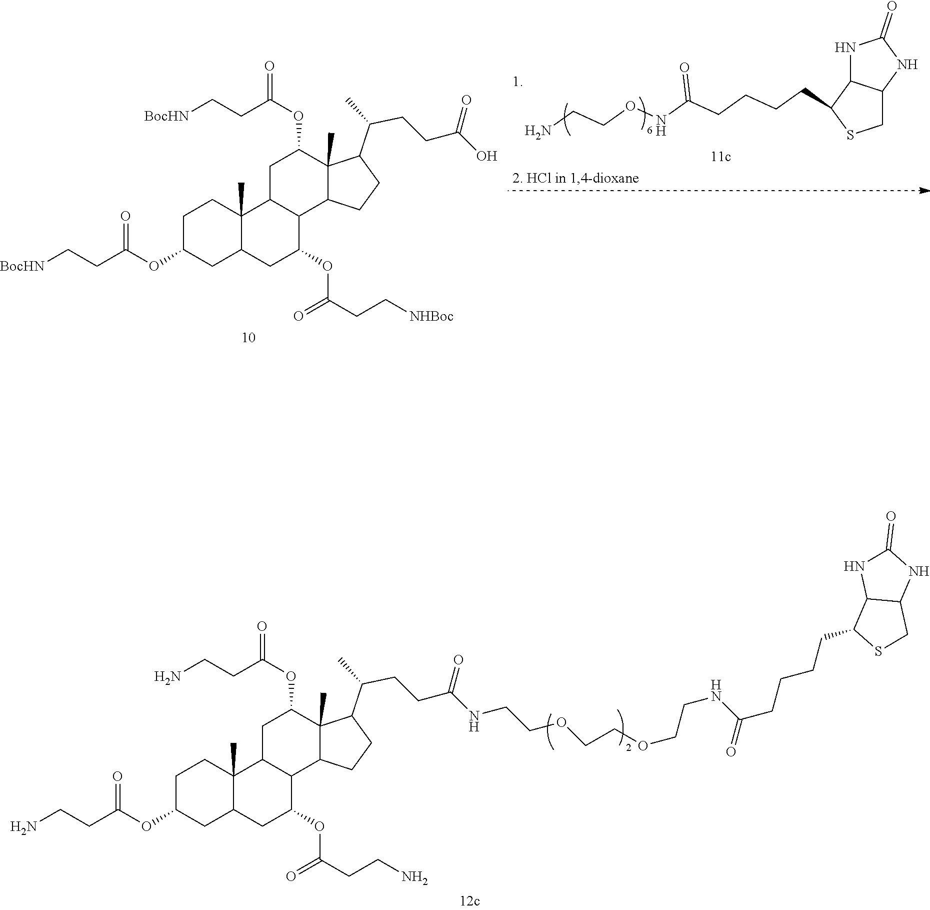

Example 6 is a contemplated preparation of (3R,7R,10S,12S,13R)-17-((2R)-5,34-dioxo-38-((4R)-2-oxohexahydro-1H-thieno[3,4-d]imidazol-4-yl)-9,12,15,18,21,24,27,30-octaoxa-6,33-diazaoctatriacontan-2-yl)-10,13-dimethylhexadecahydro-1H-cyclopenta[a]phenanthrene-3,7,12-triyl tris(3-aminopropanoate) (12d). Compound 11 from Scheme 2 may be replaced with compound 11d with the same conditions to produce a longer chain linkage between the biotin and the remaining amine to generate compound 12d.

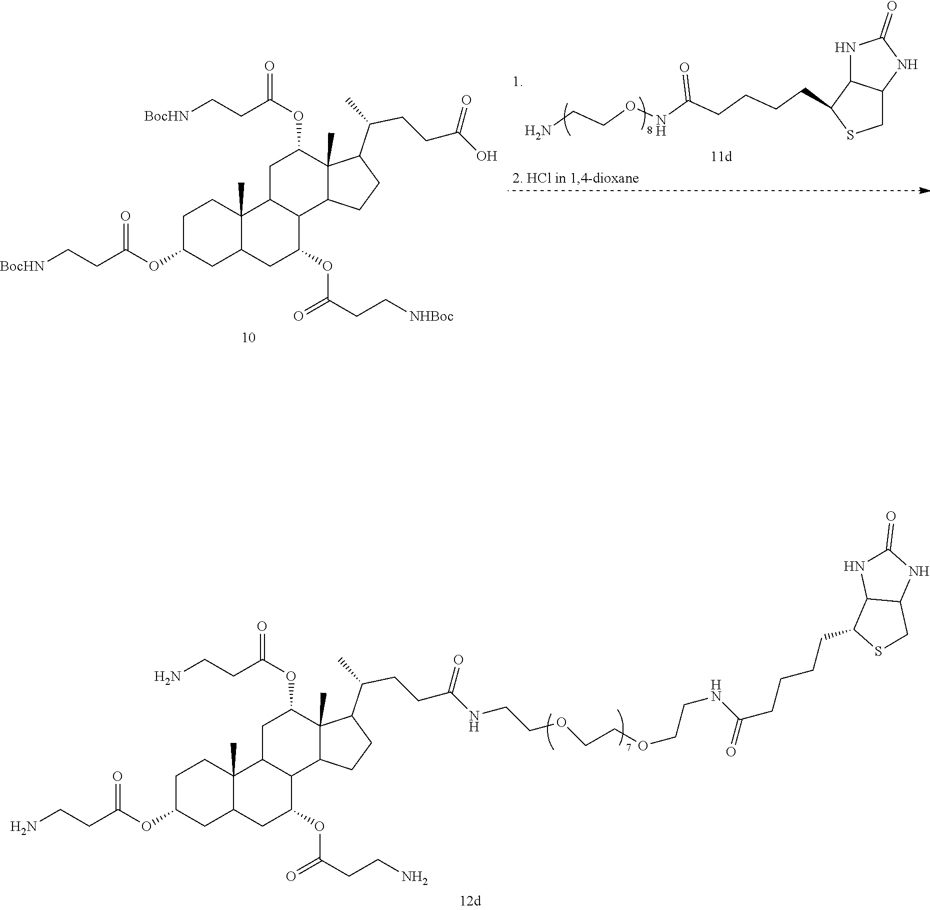

Example 7 is a contemplated preparation of (3R,7R,10S,12S,13R)-17-((2R)-5,79-dioxo-83-((4R)-2-oxohexahydro-1H-thieno[3,4-d]imidazol-4-yl)-9,12,15,18,21,24,27,30,33,36,39,42,45,48,51,54,57,60,63,66,69,72,75-tricosaoxa-6,78-diazatrioctacontan-2-yl)-10,13-dimethylhexadecahydro-1H-cyclopenta[a]phenanthrene-3,7,12-triyl tris(3-aminopropanoate) (12e). Compound 11 from Scheme 2 may be replaced with compound 11e with the same conditions to produce a longer chain linkage between the biotin and the remaining amine to generate compound 12e.

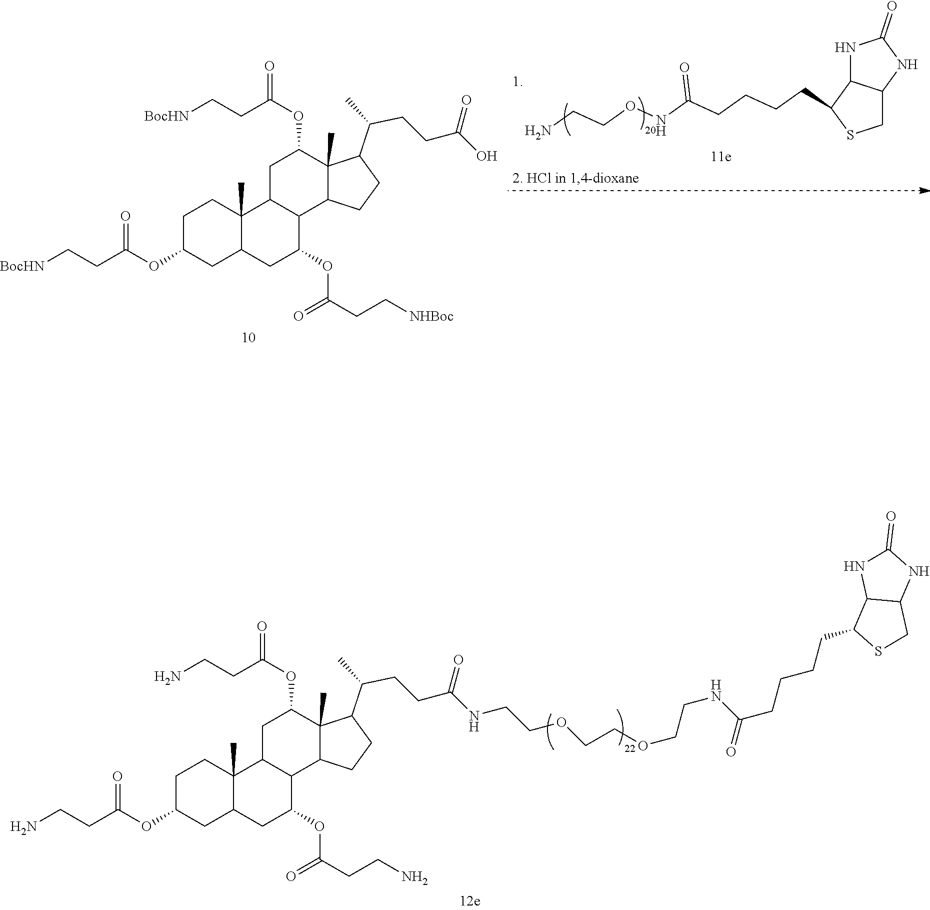

Example 8 is a contemplated preparation of (3R,7R,9S,10S,12S,13R,14S,17S)-17-(4-(((1-(2-(2-(2-(5-((3aS,4S,6aR)-3a,6a-dimethyl-2-oxohexahydro-1H-thieno[3,4-d]imidazol-4-yl) pentanamido) ethoxy) ethoxy)ethyl)-1H-1,2,3-triazol-4-yl)methyl) (octyl)amino)-4-oxobutyl)-10,13-dimethylhexadecahydro-1H-cyclopenta[a]phenanthrene-3,7,12-triyl tris(3-(chloro-15-azaneyl) propanoate) (6b). Compound 4 from Scheme 1 may be replaced with an alternate azide-terminated biotin 4b with the same reaction conditions to produce compound 6b.

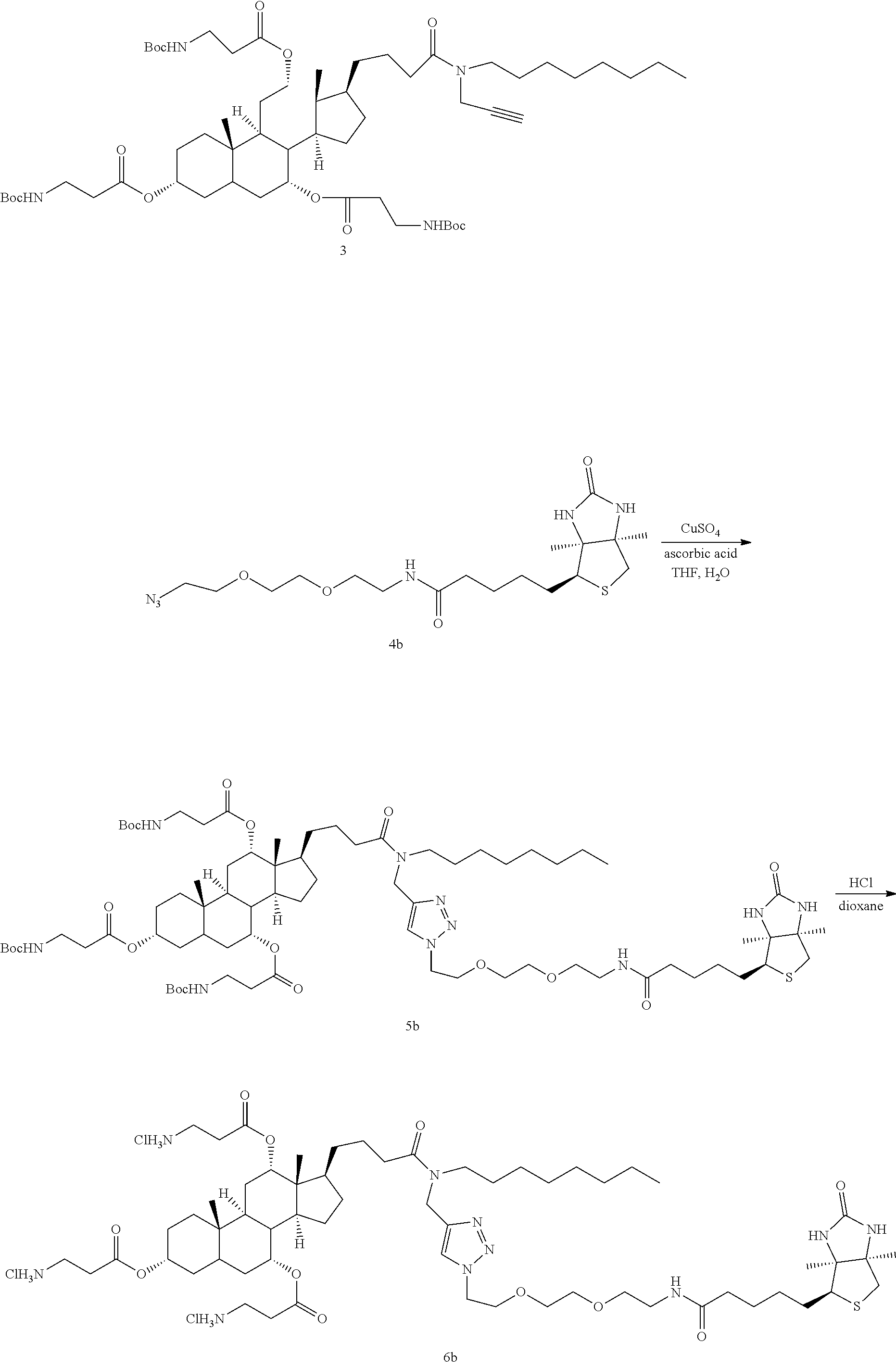

Example 2 is a contemplated preparation of (3R,7R,9S,10S,12S,13R,14S,17S)-17-(4-(((1-(2-(2-(2-(5-((3aS,4S,6aR)-3a,6a-dimethyl-2-oxohexahydro-1H-thieno[3,4-d]imidazol-4-yl) pentanamido) ethoxy) ethoxy)ethyl)-1H-1,2,3-triazol-4-yl)methyl)amino)-4-oxobutyl)-10,13-dimethylhexadecahydro-1H-cyclopenta[a]phenanthrene-3,7,12-triyl tris(3-(chloro-15-azaneyl) propanoate) (6c). Compound 4 from Scheme 1 may be replaced with an alternate azide-terminated biotin 4b while compound 1 is replaced with 1a to generate 3a with the same reaction conditions to produce compound 6c.

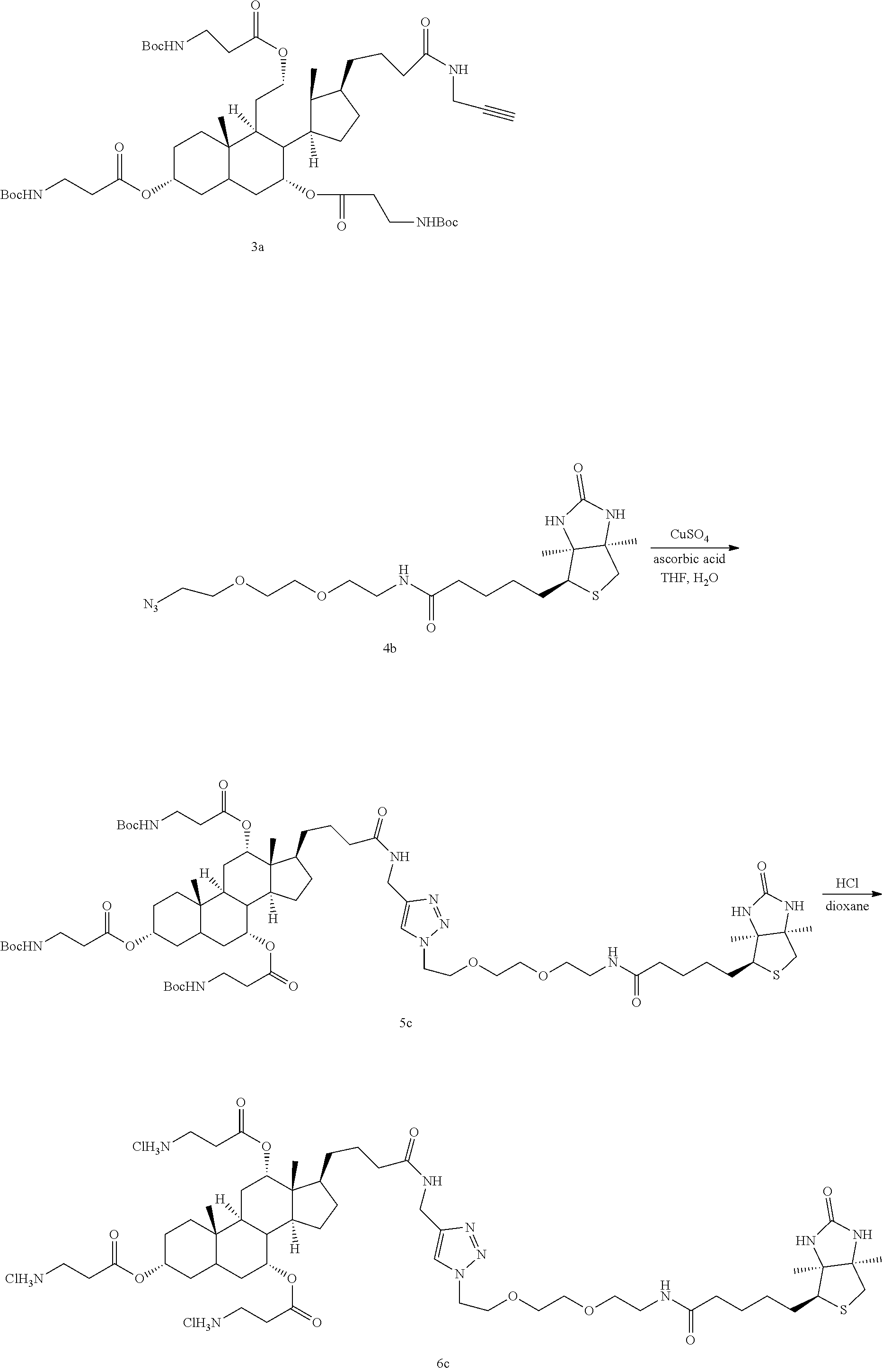

### Desthiobiotin MCAs

Example 10 is a contemplated preparation of (3R,7R,9S,10S,12S,13R,14S,17S)-10,13-dimethyl-17-(4-(octyl((1-(2-(2-(2-(6-((R)-4,5,5-trimethyl-2-oxoimidazolidin-4-yl) hexanamido) ethoxy) ethoxy)ethyl)-1H-1,2,3-triazol-4-yl)methyl)amino)-4-oxobutyl) hexadecahydro-1H-cyclopenta[a]phenanthrene-3,7,12-triyl tris(3-(chloro-15-azaneyl) propanoate) (6d). Compound 4 from Scheme 1 may be replaced with a desthiobiotin derivative 4d. The desthiobiotin will react with the amino azide to generate the MCA compound 6d.

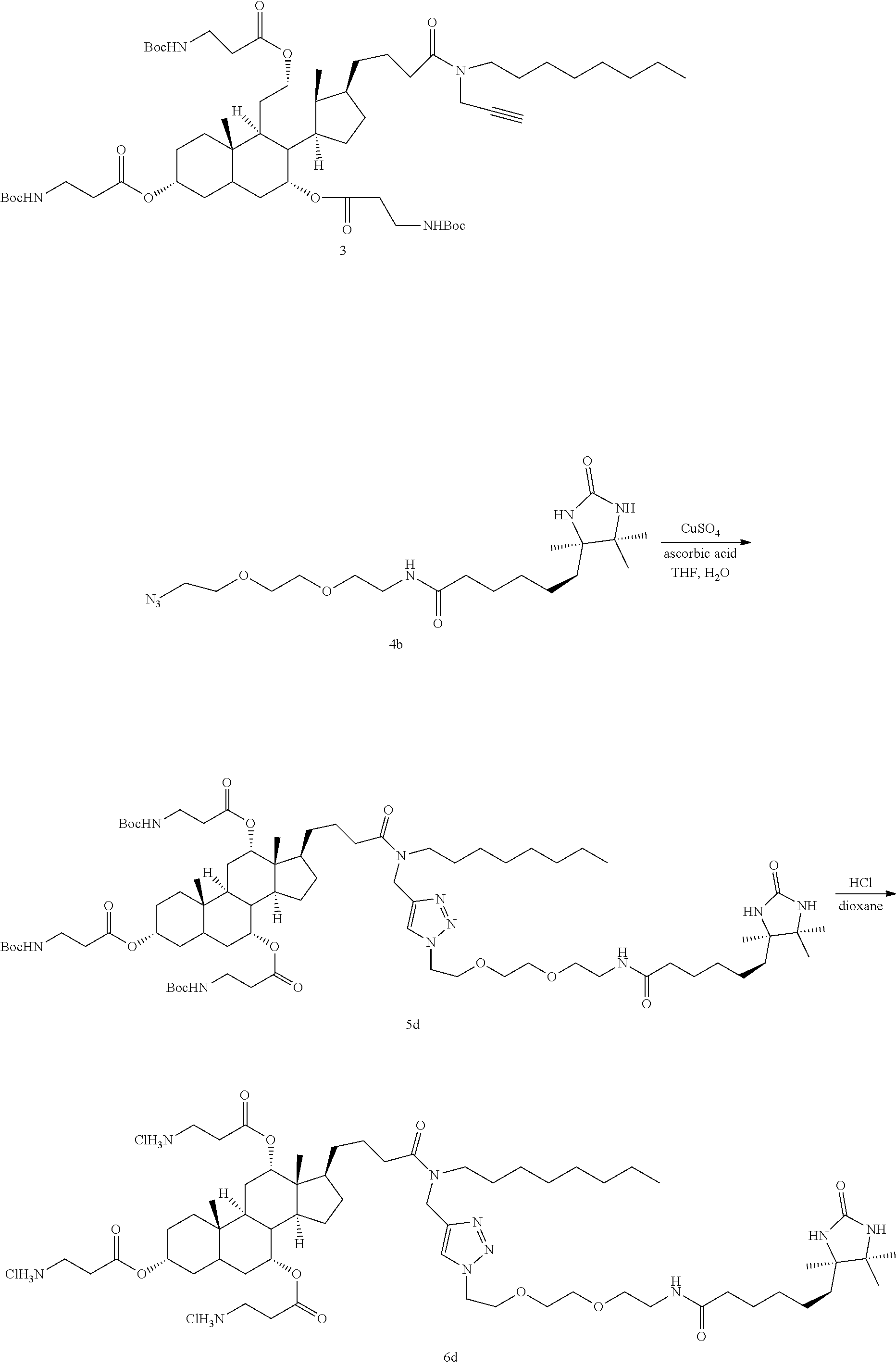

Example 11 is a contemplated preparation of (3R,7R,10S,12S,13R)-10,13-dimethyl-17-((2R)-21-((4R)-5-methyl-2-oxoimidazolidin-4-yl)-5,16-dioxo-9,12-dioxa-6,15-diazahenicosan-2-yl) hexadecahydro-1H-cyclopenta[a]phenanthrene-3,7,12-triyl tris(3-aminopropanoate) (12e). Scheme 1 may be used to generate compound 12e via reacting the desthiobiotin with a diamine to produce compound 11e and reacting that with compound 10 from Scheme 1 to form compound 12e.

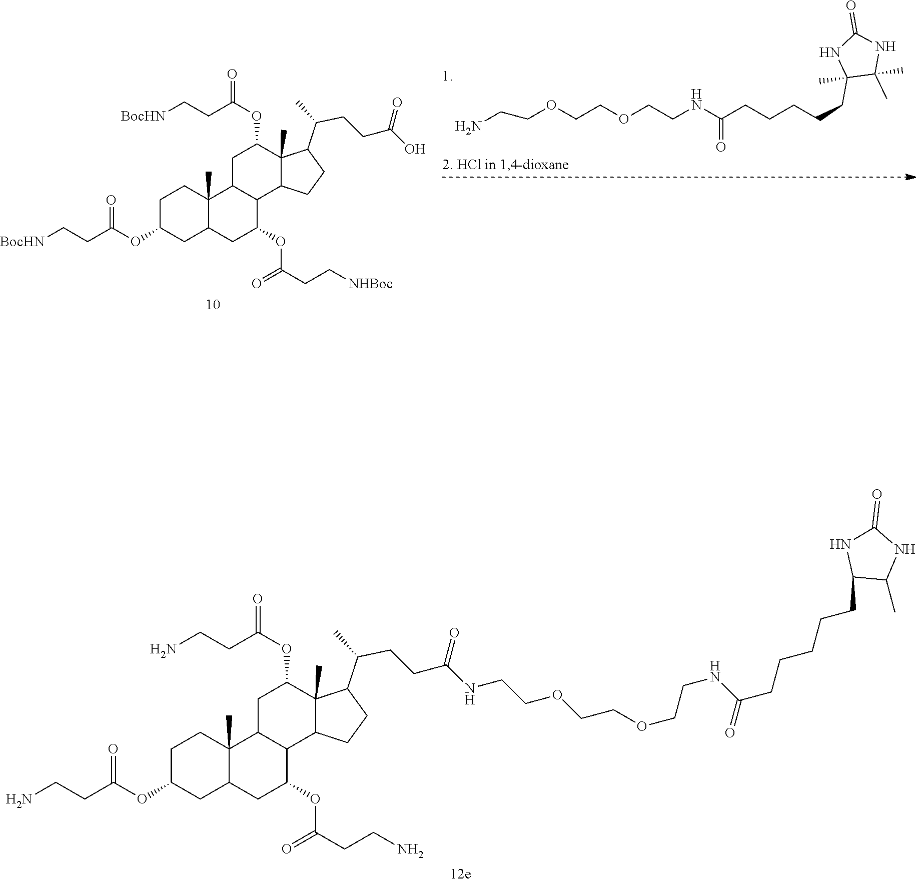

Example 12 is a contemplated preparation of (3R,7R,9S,10S,12S,13R,14S,17S)-10,13-dimethyl-17-(4-oxo-4-(((1-(2-(2-(2-(6-((R)-4,5,5-trimethyl-2-oxoimidazolidin-4-yl) hexanamido) ethoxy) ethoxy)ethyl)-1H-1,2,3-triazol-4-yl)methyl)amino)butyl) hexadecahydro-1H-cyclopenta[a]phenanthrene-3,7,12-triyl tris(3-(chloro-1,5-azaneyl) propanoate) (6e). Following Scheme 1, biotin may be replaced with a desthiobiotin derivative 4b. The desthiobiotin will react with the amino azide to generate the MCA compound 6e.

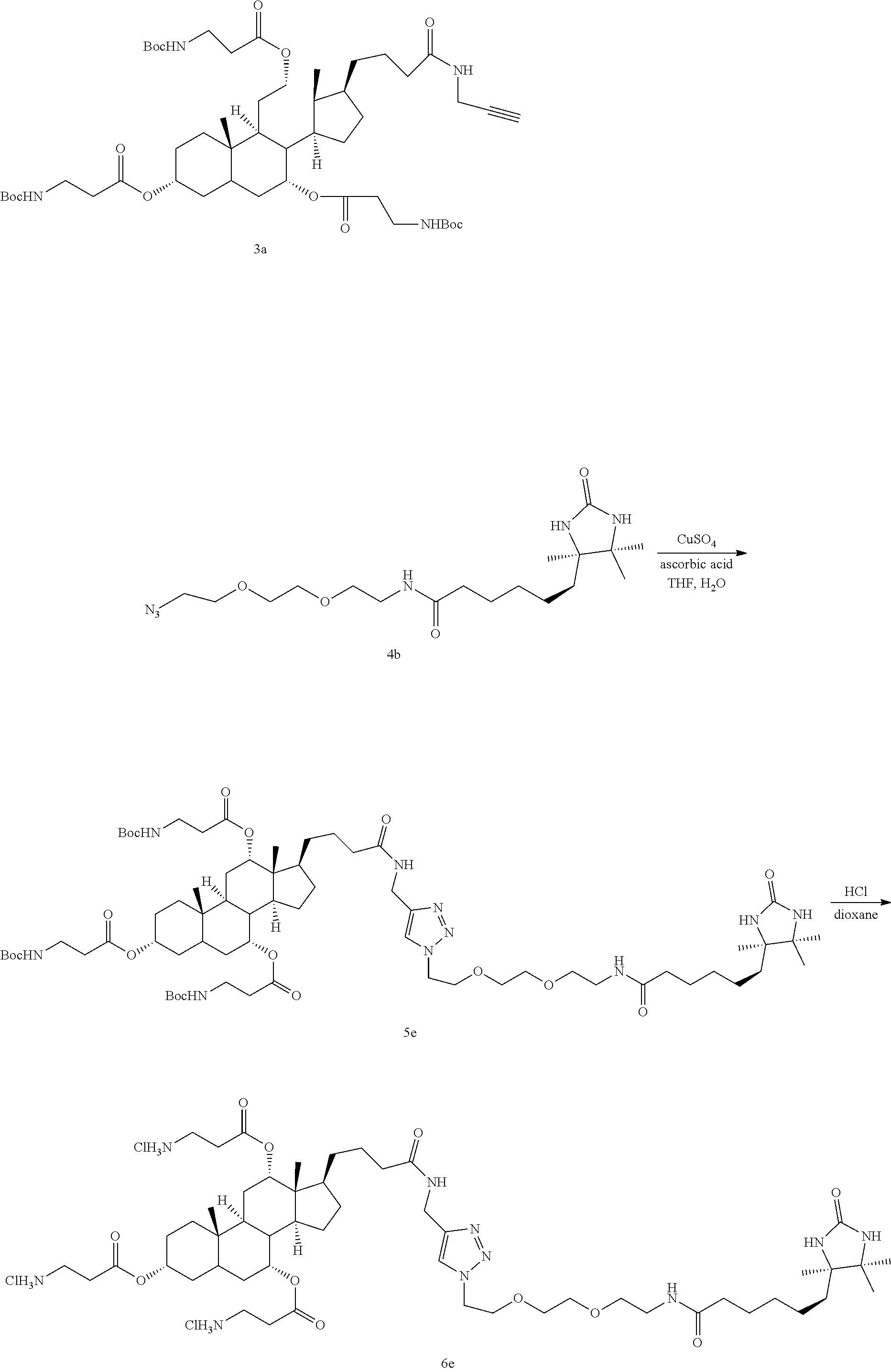

### Peptide-Based MCAs

In some examples, short sequence peptides (6-20 amino acids) containing His-Pro-Gin-Asn (HPQN) motifs within the sequence may be used to generate a peptide-based MCA. In some examples, the N-terminus of the peptide may be left as a free amine for attachment to another molecule such as a linking group, via the activated acid chemistry previously discussed.

Example 13 is a contemplated preparation compound 6f. Compound 4 from Scheme 1 may be replaced with a Linker-HPQN derivative 4f. The Linker-HPQN may react via direct attachment of the peptide 4f N-terminus to the MCA.

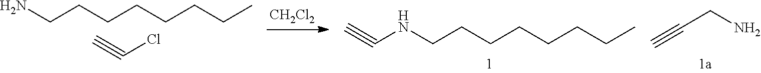

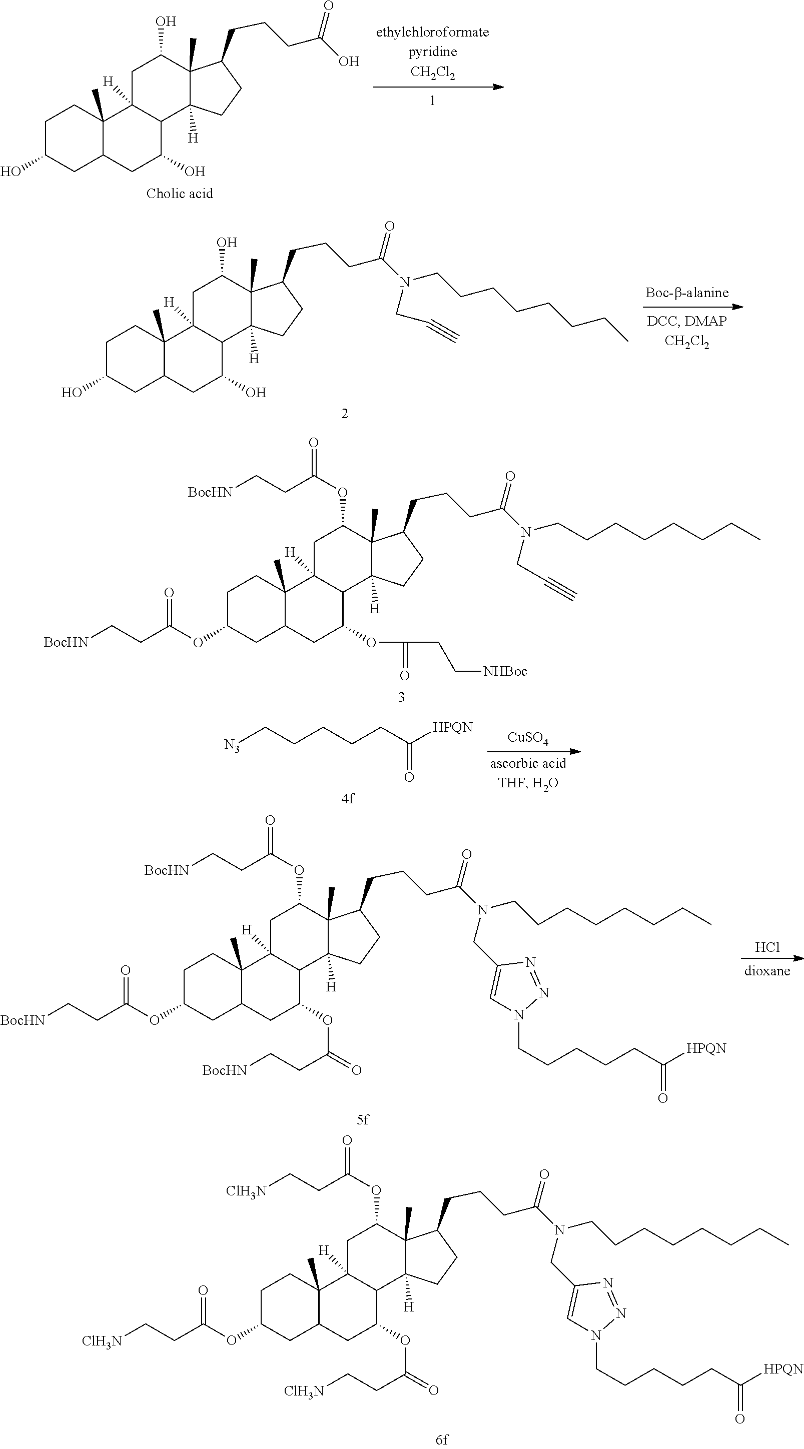

Exemple 14 is a contemplated preparation of compound 12f: Compound 11 from Scheme 2 may be replaced with a Linker-HPQN derivative. The Linker-HPQN may be prepared via direct attachment of the aminohexanoic acid to the peptide N-terminus and reacted with the MCA to form compound 12f.

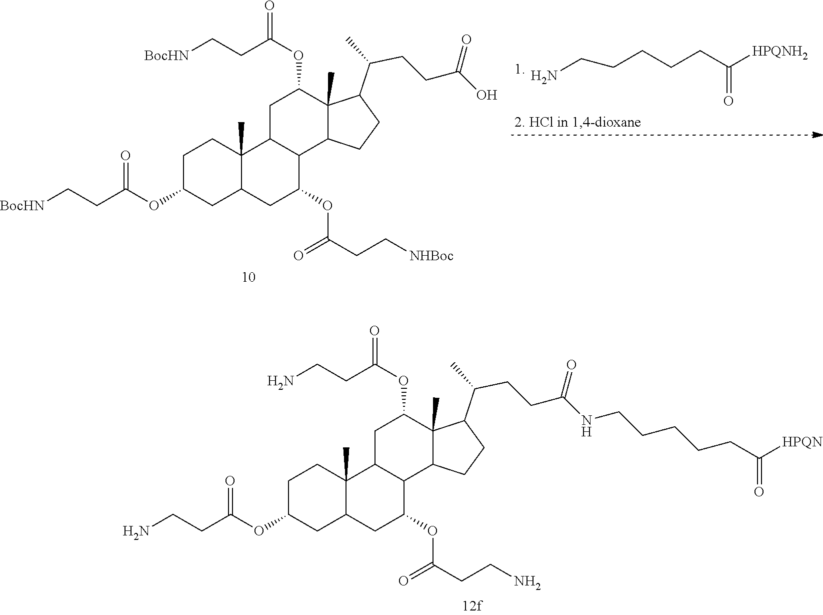

Example 15 is a contemplated preparation compound 6g: Compound 4 in Scheme 1 may be replaced with a Linker-HPQN derivative 4g while compound 1 is replaced with compound 1a to remove the octyl group. The Linker-HPQN 4g may be prepared via direct attachment of azidohexanoic acid to the peptide N-terminus and reacted with the MCA to form compound 6g.

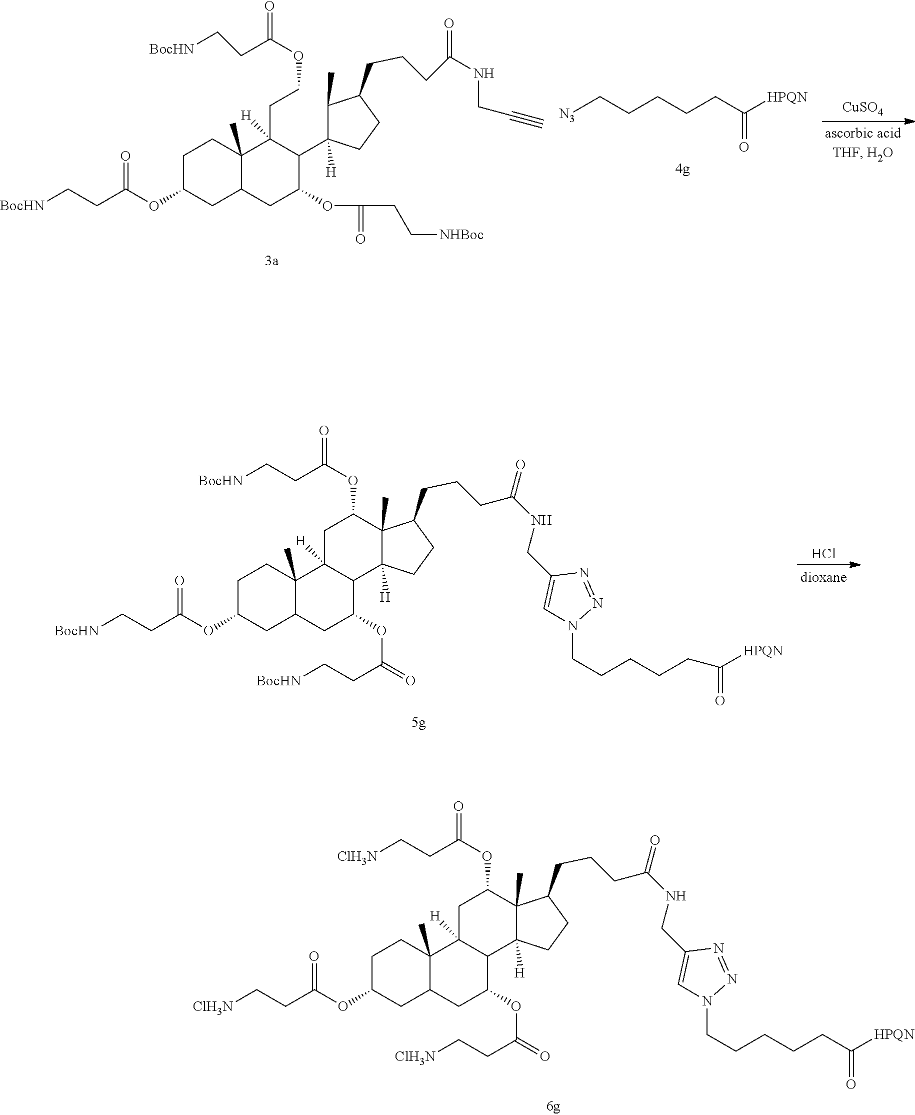

Example 16 is a contemplated preparation of compound 6h: Compound 4 from Scheme 1 may be replaced with a Linker-decapeptide derivative 4h with a sequence Ser-Ala-Trp-Arg-His-Pro-Gln-Phe-Gly-Gly (SAWRHPQGG). The Linker-SAWRHPQGG may be reacted with the MCA to form compound 6h.

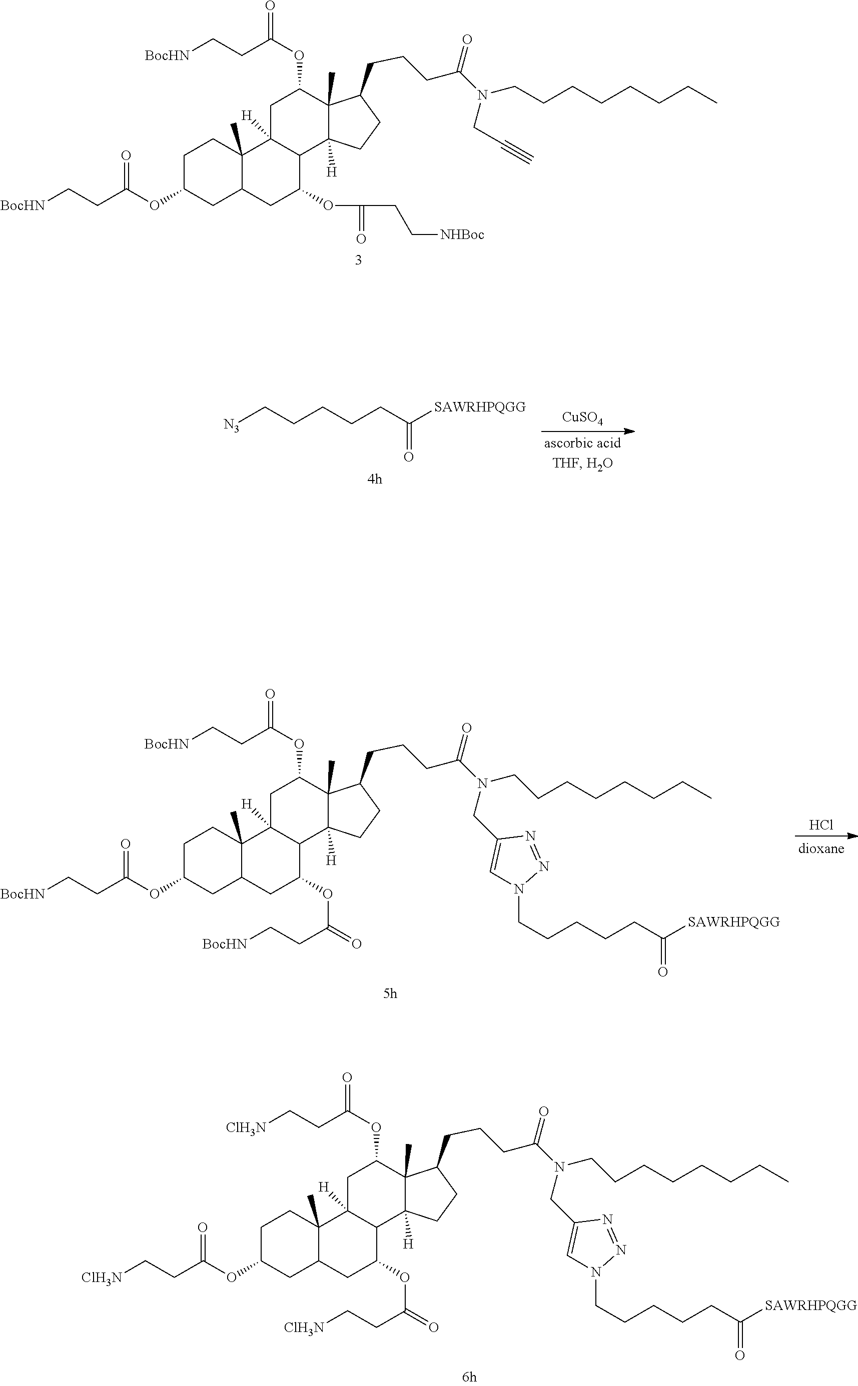

Example 17 is a contemplated preparation of compound 12g: Compound 11 from Scheme 2 may be replaced with a Linker-decapeptide derivative, for example, with a sequence Ser-Ala-Trp-Arg-His-Pro-Gin-Phe-Gly-Gly (SAWRHPQGG). The Linker-HPQN maybe reacted with the MCA to form compound 12g.

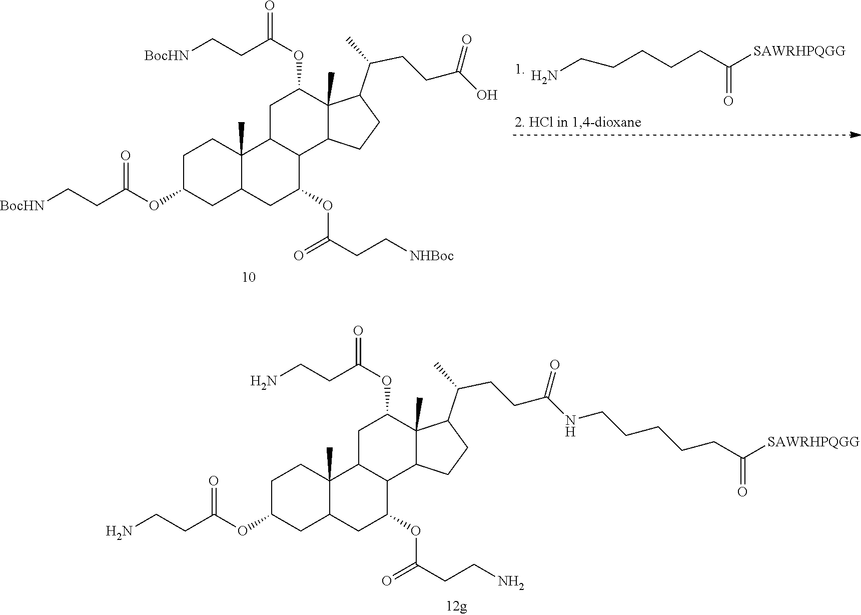

Example 18 is a contemplated preparation of compound 6i: Compound 4 in Scheme 1 may be replaced with a Linker-decapeptide derivative 4h, for example, with a sequence Ser-Ala-Trp-Arg-His-Pro-Gln-Phe-Gly-Gly (SAWRHPQGG) while compound 1 is replaced with compound 1a to remove the octyl group for generate 3a. The Linker-SAWRHPQGG will react via direct attachment of the peptide 4g N-terminus to the MCA to form compound 6i.

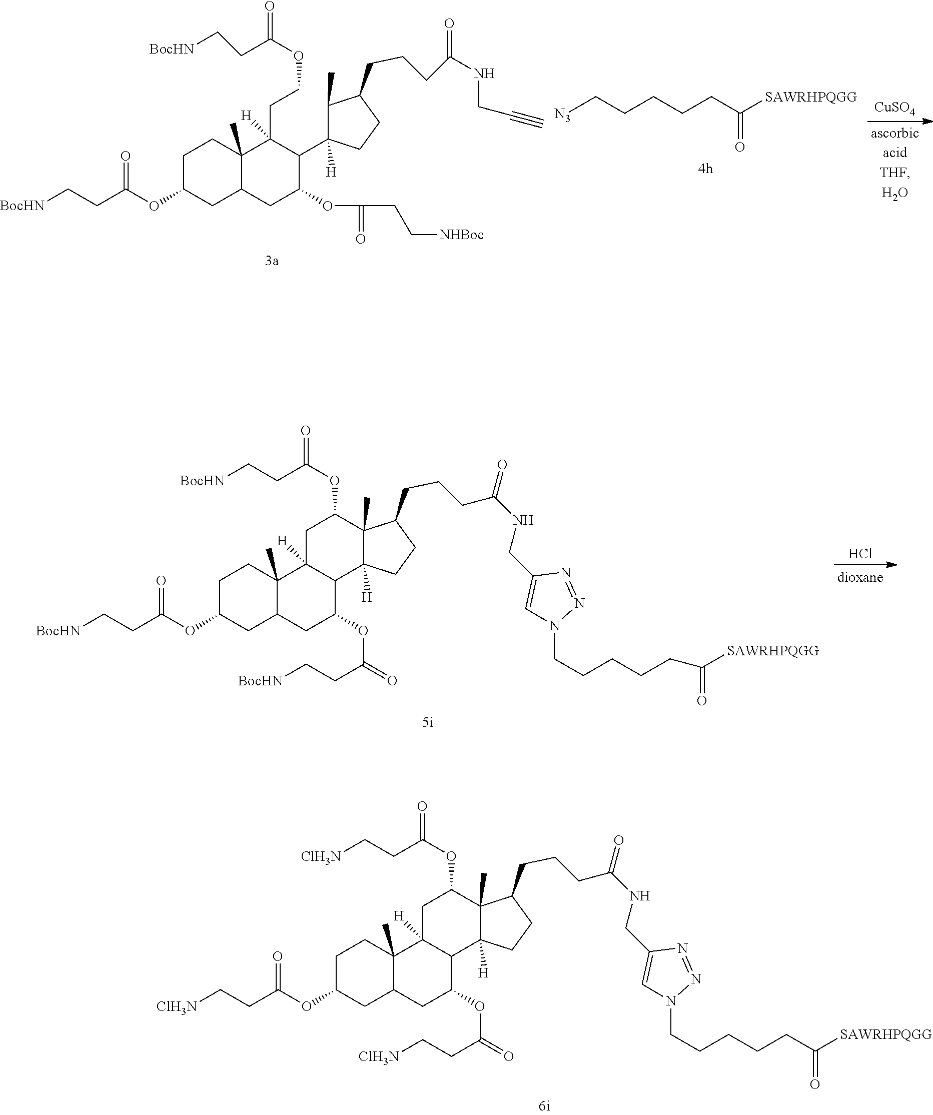

Example 19 illustrates the biological activity of Compounds 6 and 12.

Table 4 compares the structure of Compounds 6 and 12. Table 5 lists their bioactivities.

TABLE 4

| Molecule | Structure |
|---|---|
| Compound 12, Scheme II | 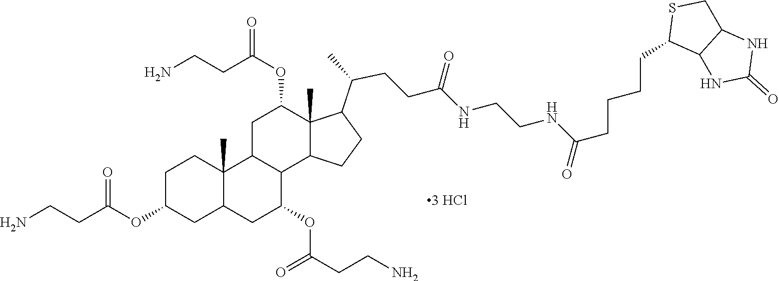 [SMILES: C[C@H](CCC(=O)NCCNC(=O)CCCC[C@@H]1SCC2NC(=O)NC21)C1CCC2C3C(C[C@H](OC(=O)CCN)[C@@]21C)[C@@]1(C)CC[C@@H](OC(=O)CCN)CC1C[C@H]3OC(=O)CCN] |

TABLE 5

| Molecule | CC₅₀ (mg/mL) | EC₅₀ (mol/L) | TI |
|---|---|---|---|
| Compound 12, | 65 | 3 | 21.6 |
| Scheme II | | | |

In the above, CC₅₀ is 50% cytotoxic concentration; EC₅₀, is 50% effective/inhibitory concentration and TI is the Therapeutic Index/Ratio. The higher the TI value, the better because this ratio quantifies the safety of the active agent.

It is believed that virus inactivation occurs via disruption of viral synthesis once inside the cell by preventing the migration of the protein from the cell organelles (i.e., Golgi apparatus, Endoplasmic Reticulum) and thus, depriving the virus from the protein needed for synthesis. Inactivation may occur via protonated amino groups (NH₃+) attacking negatively charged RNA or DNA of the viruses. As an example, the virus could be a non-envelope virus, such as CVRS3 Coxsackie virus. As an example of lipid-enveloped viruses such as Human Coronavirus, the NH₃+ may attack the viral coat assembly and thus the virus will be defective and cannot leave the cell. It is believed that virus inactivation can occur by inhibiting the production of viral proteases (e.g., Mpro and PLpro), which induce cleavage and capsid protein. This is likely to be the case for deactivating enveloped and nonenveloped viruses.

It is believed that conjugation to biotin results in reduced cytotoxicity because the latter has its own transport system (Sodium Dependent Multivitamin Transporter or SMVT) through the cell membrane, thereby bypassing impairment to the membrane. There are several existing ways for molecules to go through without disruption of the cell membrane. For instance, CSA-44 may enter the cell either by diffusing through the membrane channel, or by disrupting the phospholipid layer. In the latter case, this mode of entry may cause the cell to leak internal organelles and ultimately be destroyed. The conjugation process may influence the functional amine or/and alkyl group by steric hindrance and thus, may reduce the chance of contact with the phospholipid layer.

Example 20 illustrates the determination of cytotoxicity of CSA-44 (Comparative) in human primary cell cultures.

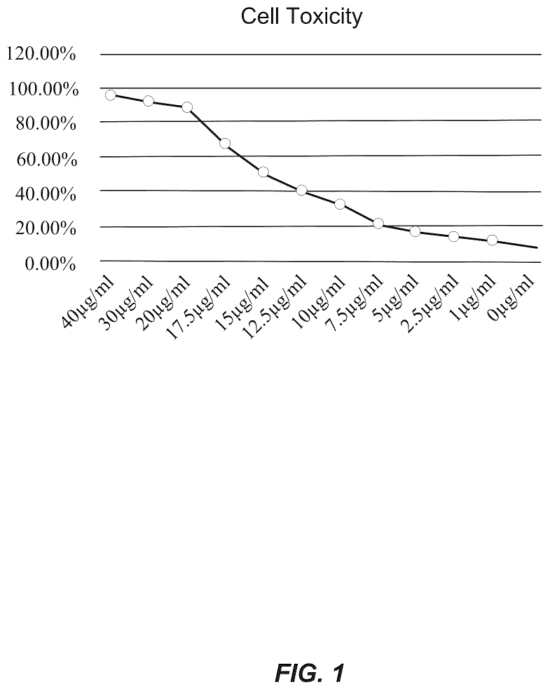

(1) HFF (human foreskin fibroblast) cells (2×105) were seeded with 2 ml of DMEM medium supplemented with 10% FBS in each well of two 12-well tissue culture plates. (2) Cells were cultured for 3 days. CSA-44 was added to test for cell toxicity. Eleven concentrations were tested: 40 μg/ml, 30 μg/ml, 20 g/ml, 17.5 μg/ml, 15 μg/ml, 12.5 μg/ml, 10 μg/ml, 7.5 μg/ml, 5 μg/ml, 2.5 μg/ml, and 1 μg/ml. Samples with concentration of 0 μg/ml were used as the control. Each concentration was tested in duplicates with two wells. (3) Cells were cultured for 48 hours with CSA-44. Trypan Blue Staining was used to determine the numbers of living and dead cells on a hemocytometer. Percentages of living and dead cells in each sample were calculated. Results suggest that the value of cytotoxic concentration CC₅₀ (the concentration of an active that reduces cell viability by 50%) of CSA-44 is around 15 μg/ml as shown in FIG. 1.

Example 21 illustrates the therapeutic activities of CSA-44 (Comparative) against human coronavirus 229E.

(1) HFF cells (5×104) were seeded with 1 ml of DMEM media supplemented with 10% FBS in each well of four 24-well tissue culture plates. (2) Cell were cultured for 3 days until HFF cells in each well attained 90% confluency (media was changed 1 day before infection). 10 μl of the 10-time diluted original solution of coronavirus, about 1×105 PFU were added into each well. Infection was allowed to proceed overnight. (3) The media was changed 24 hours post infection. CSA-44 was added to test their anti-viral effects. Ten different concentrations of the drug were tested. They were 15 μg/ml, 10 μg/ml, 9 μg/ml, 8 μg/ml, 7 μg/ml, 6 μg/ml, 5 μg/ml, 4 μg/ml, 3 μg/ml, and 2 μg/ml with 0 μg/ml as the control. Each concentration has 12 wells for test. Cells were cultured for two weeks, and cell viability was quantified. CSA-44 was effective in inhibiting viral replication and growth. The values of EC₅₀ (where EC is the concentration of the active that gives 50% of the maximal response) of CSA-44 is identified as 4 μg/ml.

Example 22 illustrates therapeutic activities of CSA-44 (Comparative against human coronavirus

(1) HFF cells (5×104) were seeded with 1 ml of DMEM media supplemented with 10% FBS in each well of four 24-well tissue culture plates. (2) Cells were cultured for 3 days until HFF cells in each well attained 90% confluency (media was changed 1 day before infection). Ten μl of the 10-time diluted original solution of coronavirus, about 1×105 PFU, were added into each well. Infection was allowed to proceed overnight. (3) The media was changed 24 hours post infection. Different concentrations of CSA-44 were added to test their anti-viral effects. Each concentration has 12 wells for test. Cells were cultured in the presence of CSA-44 for three weeks and cell viability was quantified. The results showed that CSA-44

| Compound | Luminescence intensity | | | % Luminescence | | |
|---|---|---|---|---|---|---|
| (uM) | Repeat1 | Repeat2 | Repeat3 | Repeat1 | Repeat2 | Repeat3 |
| 0 | 27,787 | 27,136 | 26,701 | 102 | 100 | 98 |
| 20 | 27,723 | 26,322 | 27,521 | 102 | 97 | 101 |
| 26.7 | 27,962 | 26,310 | 26,857 | 103 | 97 | 99 |
| 35.6 | 28,560 | 28,084 | 28,199 | 105 | 103 | 104 |
| 47.5 | 29,946 | 28,451 | 28,177 | 110 | 105 | 104 |
| 63.3 | 30,079 | 29,936 | 28,795 | 111 | 110 | 106 |
| 84.4 | 29,975 | 29,736 | 29,345 | 110 | 109 | 108 |
| 112.5 | 29,458 | 28,788 | 28,587 | 108 | 106 | 105 |
| 150 | 29,194 | 28,202 | 28,754 | 107 | 104 | 106 |
| 200 | 30,150 | 29,181 | 28,872 | 111 | 107 | 106 |
| Background | 111 | 109 | 89 | | | |

was effective in inhibiting viral replication and growth. The values of EC₅₀ of CSA-44 are estimated to be 4 μg/ml.

Example 23 illustrates a measure of cell proliferation of an embodiment of the present disclosure.

The effect of Compound 12 was measured on cell proliferation using Calu-3 cells (ATCC #HTB-55), a transformed type of cells frequently used for testing cytotoxicity and antiviral activity.

Cells were cultured in MEM medium with 10% FBS, and 1% Pen-strep. Cells were plated at 10,000 cells per well in white clear-bottom 96 well microplates in 50 μls of growth media. The next day compound dilutions were prepared at 2× in growth media. Fifty μls of compound dilutions were added to the cells to achieve the desired final concentration. One hundred μls of growth medium was added to cell-free control wells for determining background luminescence. Cells were incubated at 37° C. and 5% CO₂ for 3 days. At the end of the incubation, the Cell Titer Glo assay was run as directed: Add 100 μls of reagent per well and read luminescence using a luminometer (BioTek Synergy™ 2 microplate reader).

The results indicate that the CC₅₀ is >200 uM for Compound 12. The CC₅₀ is the concentration of test compound required to reduce cell viability by 50%.

Example 24 illustrates the determination of cytotoxicity of CMA Conjugate (Compound 12) in human primary cell cultures.

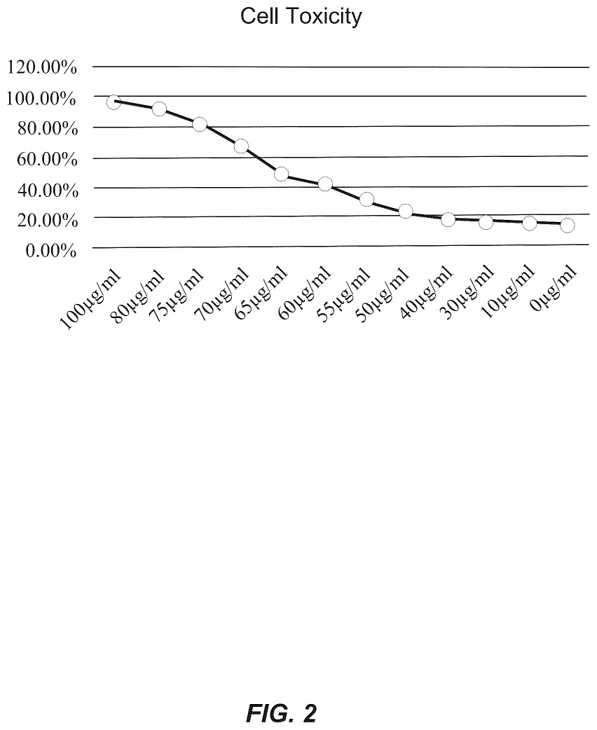

(1) HFF cells (2×105) were seeded with 2 ml of DMEM medium supplemented with 10% FBS in each well of two 12-well tissue culture plates. (2) Cells were cultured for 3 days. CMA Conjugate was added to test for cell toxicity. Eleven concentrations were tested. They were 100 μg/ml, 80 μg/ml, 60 μg/ml, 50 μg/ml, 40 μg/ml, 30 μg/ml, 25 μg/ml, 20 μg/ml, 15 μg/ml, 10 μg/ml, and 5 μg/ml. Samples with concentration of 0 μg/ml were used as the control. Each concentration was tested in duplicates with two wells. (3) Cells were cultured for 48 hours with CMA Conjugate. Trypan Blue Staining was used to determine the numbers of living and dead cells on a hemocytometer. Percentages of living and dead cells in each sample were calculated. Results suggest that the value of cytotoxic concentration CC50 (the concentration of an active that reduces cell viability by 50%) of CMA Conjugate (Compound 12) is around 65 μg/ml as shown in FIG. 2.

Example 25 illustrates a Cytotoxicity Assay of CMA conjugate (Compound 12).

Calu-3 cells were seeded in 96-well plates one day prior to testing. At 80-90% confluency, growth medium was removed and replaced with a medium containing 50 μg/mL of compound 12 dilutions. All treatments were added in quadruplicate or quintuplicate wells. Plates were incubated for 24 h before being washed and replaced with fresh untreated growth medium. MTS was added to each well as recommended by the manufacturer's protocol (CellTiter 96 Aqueous One Solution Cell Proliferation Assay, Promega, Madison, WI, USA) and incubated for 3-4 h.

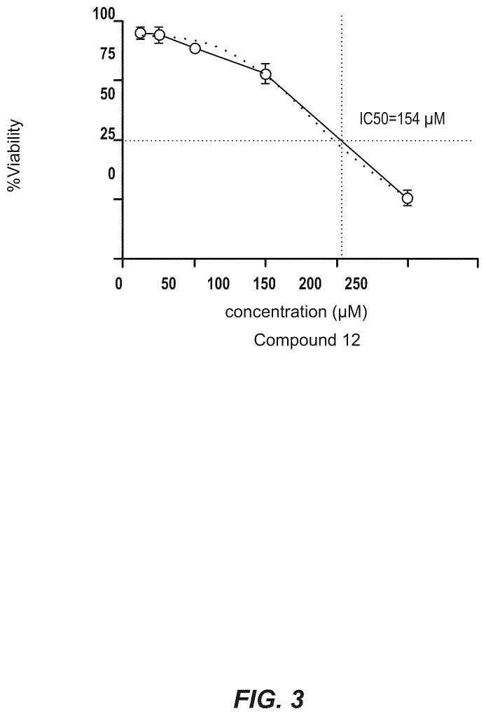

After incubation, absorbance was measured at 490 nm on a SpectraMax M5 (Molecular Devices, LLC, San Jose, CA, USA) plate reader with SoftMax Pro 6.2.1 software. FIG. 3 shows that compound 12 has a IC₅₀ (i.e., CC₅₀) of 154 μM.

Example 26 illustrates the therapeutic and prophylactic activities of CMA conjugate (Compound 12) against human coronavirus 229E.

### A. Therapeutic Activities of a CMA Conjugate (Compound 12)

(1) HFF cells (5×104) were seeded with 1 ml of DMEM media supplemented with 10% FBS in each well of four 24-well tissue culture plates. (2) Cells were cultured for 3 days until HFF cells in each well attained 90% confluency (media was changed 1 day before infection). 10 μl of the 10-time diluted original solution of coronavirus, about 1×105 PFU were added into each well. Infection was allowed to proceed overnight. (3) The media was changed 24 hours post infection. CMA conjugate was added to test their anti-viral effects. Ten different concentrations of the drug were tested. They were 15 μg/ml, 10 μg/ml, 9 g/ml, 8 μg/ml, 7 μg/ml, 6 μg/ml, 5 μg/ml, 4 μg/ml, 3 μg/ml, and 2 μg/ml with 0 μg/ml as the control. Each concentration has 12 wells for test. Cells were cultured for two weeks, and cell viability was quantified. CMA Conjugate was effective in inhibiting viral replication and growth. The values of EC₅₀ (where EC is the concentration of the active that gives 50% of the maximal response) of CMA Conjugate was identified as 3 μg/ml.

### B. Prophylactic Activities of a CMA Conjugate (Compound 12)

A series of experiments were carried out to study if cells can uptake Compound 12. In this study, human foreskin fibroblasts were used as the human primary cell culture model. Cells were incubated with 5 μg/ml of Compound 12 at different time points, then subsequently fixed and stained with avidin conjugated fluorophore, and visualized with fluorescent microscopy. Within 15 minutes, cells incubated with Compound 12 were found to contain fluorescence. The fluorescent signal remained strong after 3 hours of incubation. These results indicate that the uptake is rapid and efficient and that Compound 12 is stable in cells. Furthermore Compound 12 appears to be primarily localized in the cytoplasm, such as in the cellular membranous systems (e.g. ER, Golgi apparatus, trans-Golgi network, etc.). Similar results were also found in cells infected with human coronavirus 229E. These results suggest that Compound 12 can be internalized in the cytoplasm of human cells, including the membranous systems.

The effect of compound 12 was studied on cellular structures and organelles such as Golgi apparatus, mitochondria, and endoplasmic reticulum (ER). Treatment with Compound 12 at concentrations below the CC₅₀ values did not affect the overall structure and organization of these cellular structures and organelles. In conclusion, this study shows that (a) Compound 12 enters human cells, (b) Compound 12 appears to be primarily distributed and localized in the cytoplasm of human cells including the membranous systems such as the Golgi apparatus and the trans-Golgi networks, and (c) Compound 12 appears to have no significant or substantial effects on the overall structures and organization of numerous intracellular structures and organelles such as Golgi apparatus and ER.

Example 27 illustrates the mode of action (MOA) of an embodiment of the present disclosure.

It was determined that Compound 12 generated a high level of antiviral activity against 229E HCV after the virus infected the cells, indicating that the compounds of the present disclosure are inside the cell and are able to reduce the virus titer by targeting the released virus genetic materials and not the lipid viral envelope which is lost upon entry into the cell.

In the “disinfectant” experiments, stock solution of Compound 12 was generated by dissolving the compound in 10% DMSO solution. Different amounts of Compound 12 from the stock solution were mixed with 104 PFU of Coronavirus 229E. After incubation at room temperature for 15 minutes, the mixture of virus and Compound 12 was added to the culture of primary human foreskin fibroblasts (HFFs). At 24 hours post-infection, culture medium was changed and the infected HFFs were further cultured in the absence of Compound 12. The level of inhibition of virus infection and production was determined. Results indicate that Compound 12 has an EC₅₀ (μg/ml) of 500.

Multiple modes of action were evidenced by the (1) limited envelope disruption in the assay (above) and the (2) inhibition of 3CL protease (below).

The compound is dissolved in DMSO. A series of dilutions were prepared with 5% DMSO in 3CL Protease assay buffer. 10 μl of the dilution was added to a 50 μl reaction so that the final concentration of DMSO is 1% in all of the reactions. The compound was pre-incubated in duplicate at room temperature for 30 minutes in a mixture containing 3CL Pro assay buffer, 3CL enzyme and a test compound. After 30 minutes, the enzymatic reactions were initiated by the addition of 3CL Protease substrate. The enzymatic reaction proceeded for 4 hours at room temperature. Fluorescence intensity was measured at an excitation of 360 nm and an emission of 460 nm using a Tecan Infinite M1000 microplate reader.

3CL Protease (SARS-COV-2) Inhibitor Assay #110922: IC50>10 μM; 24% Inhibition @ 10 μM

| | 3CL Activity | | | |
|---|---|---|---|---|
| Compound 12 | (Fluorescent count) | | % Activity | |
| (μM) | Repeat 1 | Repeat 2 | Repeat 1 | Repeat 2 |
| No Compound | 4879 | 4958 | 99 | 101 |
| 0.0003 | 4878 | 4956 | 99 | 101 |
| 0.001 | 5061 | 5027 | 103 | 103 |
| 0.003 | 5028 | 5032 | 103 | 103 |
| 0.01 | 4755 | 4913 | 96 | 100 |
| 0.03 | 4690 | 4503 | 95 | 90 |
| 0.1 | 4492 | 4450 | 90 | 89 |
| 0.3 | 4543 | 4409 | 91 | 88 |
| 1 | 4475 | 4438 | 90 | 89 |
| 3 | 4157 | 4029 | 82 | 79 |
| 10 | 3849 | 3945 | 75 | 77 |
| Blank | 689 | 693 | | |

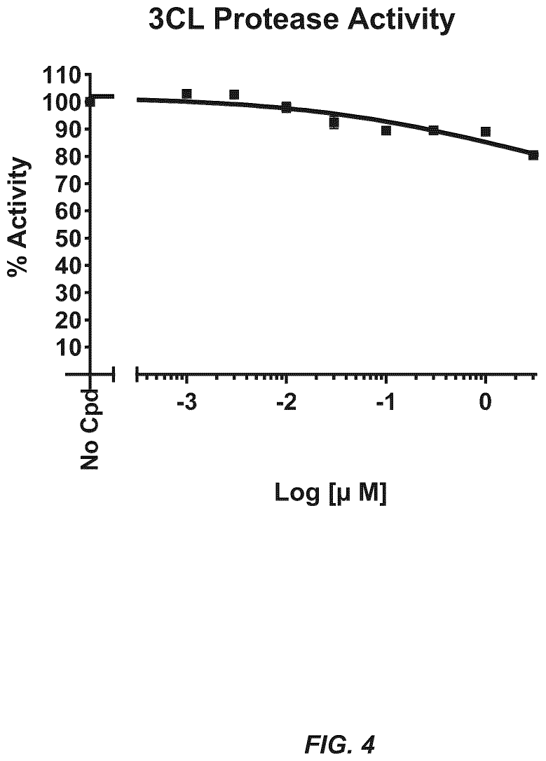

FIG. 4 shows the protease activity of Compound 12 of the present disclosure. Protease inhibitors (PIs) are antiviral drugs that act by interfering with enzymes that cleave proteins. Simply, the PI of the present disclosure prevents the virus from making copies of itself. The calculated IC₅₀>10 μM and there was 24% inhibition at 10 μM.

Example 28 illustrates the activation of TNF-α of a compound of the present disclosure.

It was confirmed that Compound 12 stimulates TNF-α production in HEK293 Cells. For instance, 5 μg/ml of Compound 12 induces the cells to generate 7,123 pg/ml of TNF-α while untreated-58.8 pg/ml.

The virus enters the cell via the angiotensin-converting enzyme-2 (ACE-2) and is sensed (essentially) by Toll-like receptor 7 (TLR7), which exists in endosomes. TLR7 activation leads to the production of alpha interferon, TNF-α, and the secretion of interleukin (IL)-12 and IL-6. This results in the formation of CD8+-specific cytotoxic T cells and, through the CD4⁺ helper T cell, leads to the formation of antigen-specific B cells and antibody production. This adaptive immune response controls viral infection and determines clinical recovery.

Example 29 illustrates the anti-viral activity of a compound of the present disclosure.

Calu3 cells were seeded into a 24-well plate until about 80% confluency. Cell media was replaced with 100 μM of Compound 12 supplemented media or equivalent amounts of vehicle solution (0.01% lactic acid) for a 1 hour incubation period. After incubation, cells were inoculated with variants WA or BA.5 strains of SARS-COV-2 virus at an MOI: 0.1 for a 1 hour adsorption period. Following absorption, cells were washed with PBS to remove virus and then given 100 μM of Compound 12 supplemented or equivalent amounts of vehicle solution media. Viral supernatants were collected 24 hours post infection (hpi) and media was re-supplemented with 100 μM of Compound 12 supplemented media or equivalent amounts of vehicle solution. Collection and drug addiction was repeated until 72 hpi.

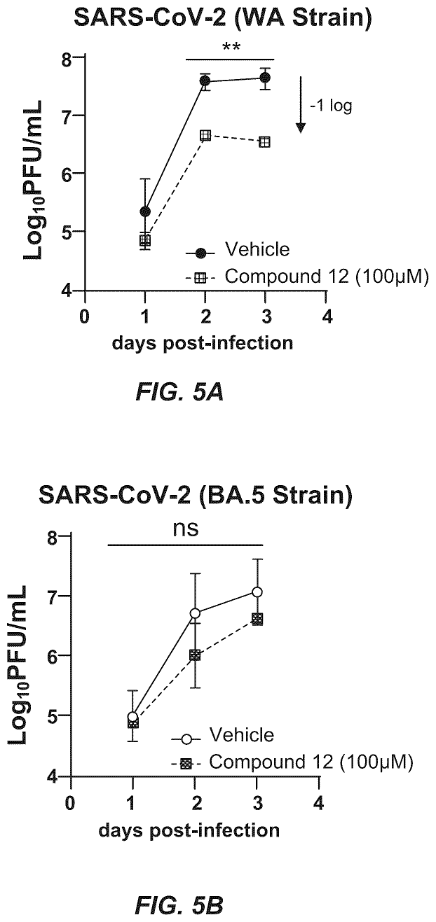

FIG. 5A shows the reduction of viral strain WA in the presence of Compound 12. FIG. 5B shows the reduction of viral strain BA.5 in the presence of Compound 12.

Example 30 illustrates the in vivo anti-viral activity of a compound of the present disclosure.

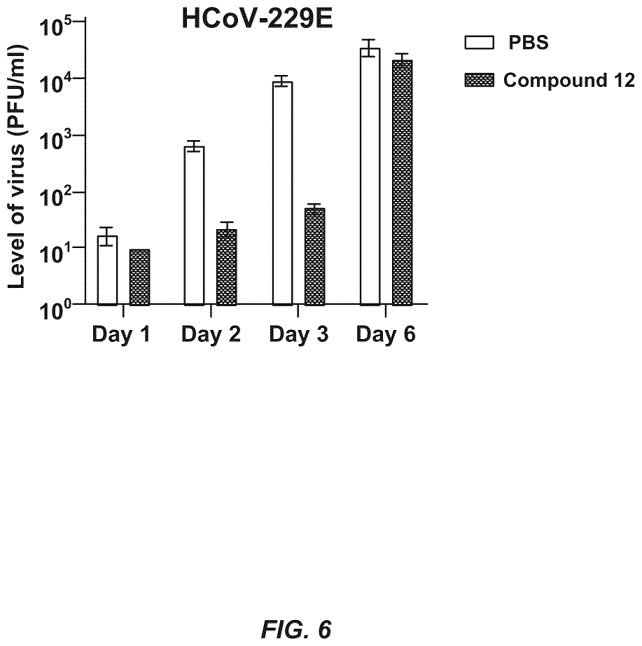

FIG. 6 illustrates the in vivo viral load reduction in mice lung tissue administered Compound 12 compared to control mice that were not administered a compound of the disclosure. In the Group administered Compound 12, a substantial (30-200 fold) reduction of virus replication and production was recorded 2 days and 3 days post-treatment. The results indicate Compound 12 inhibits HCV infection and growth in mice. The rebound seen at 6 days strongly suggests that a second dose (treatment) is needed 2-3 days post infection, or a single 1 dose treatment of 1.0 mg/ml would be efficacious.

Example 31 illustrates histopathological studies on infected and non-infected mice.

This Example uses the following Groups:

Group A: No infection, treated with PBS: Ten (10) BALB/c 6-weeks old female mice were used for this group. Mice were first anesthetized with isoflurane. Each mouse was intranasally treated with one dose of PBS (75 ul). Mice were sacrificed at 72 hrs, and 7 days post treatment. The lungs from these mice were collected for immunohistochemistry and cytokines analysis.

Group B: No infection, treated with Compound 12: Twenty (20) BALB/c 6-weeks old female mice was used for this group. Mice were first anesthetized with isoflurane. Each mouse was intranasally treated with one dose of 0.5 mg of Compound 1q2 diluted in PBS (75 ul). Mice were sacrificed at 24 hrs, 48 hrs, 72 hrs, and 7 days post treatment. The lungs from these mice were collected for immunohistochemistry and cytokines analysis.

Group C: Infection with 229E, treated with PBS: Thirty five (35) BALB/c 6-weeks old female mice were used for this group. Mice were first anesthetized with isoflurane. Each mouse was intranasally infected with 1×105 plaque forming unit (pfu) of HCoV-229E and then treated with PBS (75 ul) 24 hours later. Mice were sacrificed at 24 hrs post infection, and 24 hrs, 48 hrs, 72 hrs, 6 days, and 7 days post treatment. The lungs from these mice were collected for viral load, immunohistochemistry and cytokines analysis.

Group D: Infection with 229E, treated with Compound 12: Forty Five (45) BALB/c 6-weeks old female mice were used for this group. Mice were first anesthetized with isoflurane. Each mouse was intranasally infected with 1×105 pfu of HCoV-229E and then treated with 0.5 mg of Compound 12 diluted in PBS (75 ul) 24 hours later. Mice from this group were sacrificed at 24 hrs, 48 hrs, 72 hrs, 6 days and 7 days post treatment. The lungs from these mice were collected for viral load, immunohistochemistry and cytokines analysis.

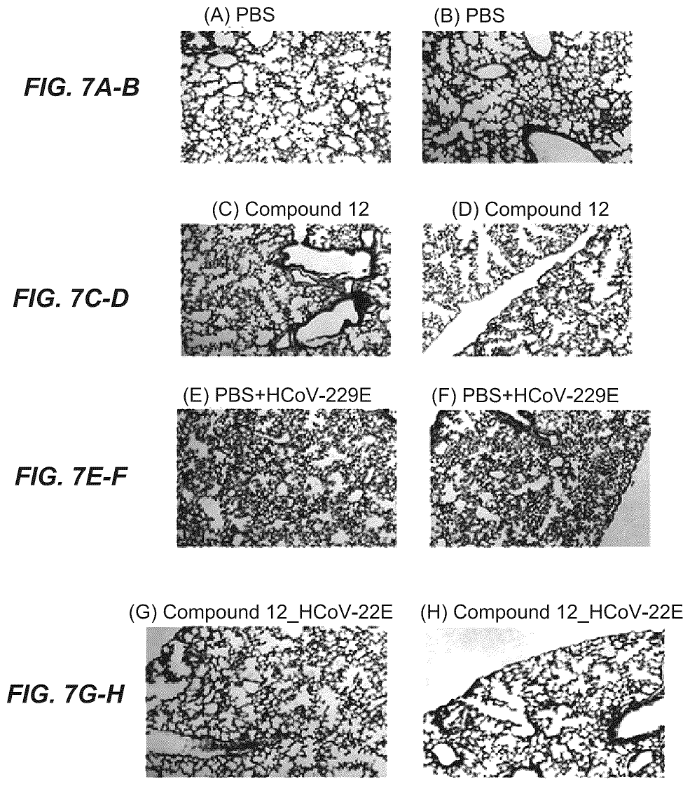

H&E staining revealed that lung tissues from the non-infected mice show normal morphology (Group A (FIGS. 7A-B); Group B (FIGS. 7C-D) 3 days post treatment. As for tissues from Group D (Infected mice treated with Compound 12), normal lung morphology (FIGS. 7G-H) was observed, whereas Group C (infected mice treated with PBS), the mice exhibited structural changes in the lungs (FIGS. 7E-F), consistent with HCV infection.

Staining and imaging results show successful delivery of Compound 12 in the lung tissues, which was detected at 1-2 days post treatment but not in 3 days post treatment. Administration of Compound 12 does not cause significant detrimental effects on the morphology of the lung at 3 days post treatment. These results indicate that Compound 12 does not exhibit significant toxicity in vivo under the dosage used (0.5 mg/mice). The results also show subtle changes of lung structure, associated with early signs of viral infection in Group C mice and relative normal lung morphology in Group D mice. These results imply antiviral activity and the beneficial effects of treatment.

Example 32 illustrates an embodiment of the present disclosure modulated cytokines as an immunomodulator.

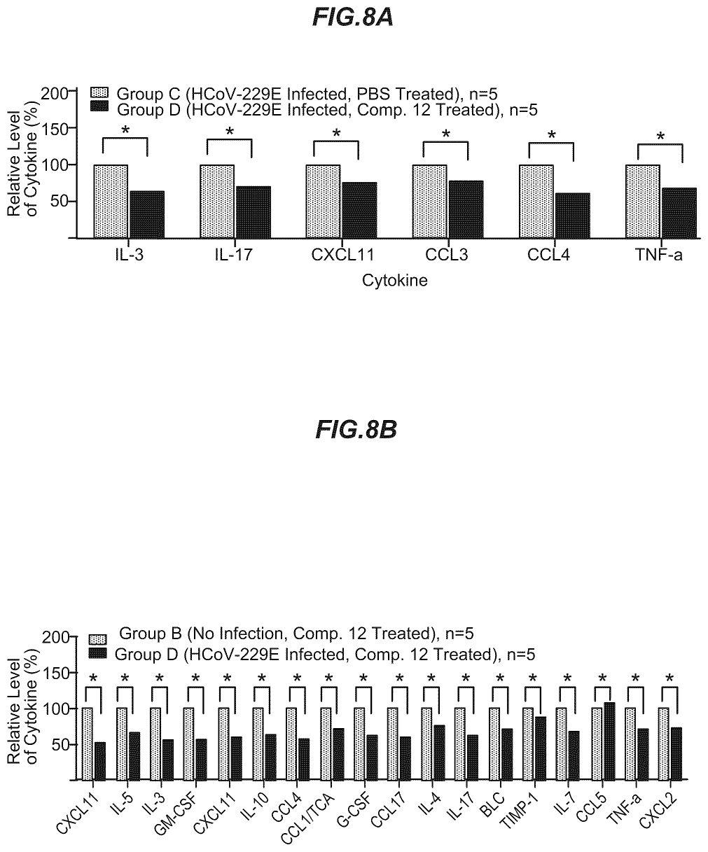

FIG. 8A shows a comparison of Group C (control) with Group D (treated with Compound 12). There is significant reduction in the following cytokines: IL-3, IL-17, CXCL11, CCL3, CCL4, and TNFα with treatment. The compounds of the present disclosure modulate cytokines and chemokines to reduce cytokine storms, sepsis, and other immune-system mediated responses such as autoimmune disorders.

FIG. 8B shows a comparison of Group B (No infection, treated with Compound 12) with Group D (HCoV-229 Infected, treated with Compound 12) wherein significant changes in relative levels of CCL11, IL-5, IL-3, GM-CSF, CXCL11, IL-10, CCL4, CCL1/TCA, G-CSF, CCL17, IL-4, IL-17, BLC, TIMP-1, IL-7, CCL5, TNF-α, CXCL2 occurs.

Example 33 illustrates the fungicidal and bactericidal activity of an embodiment of the present disclosure.

The Table below shows that Compound 12 is active against yeast and bacteria.

| MIC (μg/ml) | | | MBC (μg/ml) | | |
|---|---|---|---|---|---|
| Candida | | | Candida | | |
| albicans | MRSA | Pseudomonas | albicans | MRSA | Pseudomonas |
| Yeast | Gram+ | Gram− | Yeast | Gram+ | Gram− |
| 66.25 | 530 | 530 | 66.25 | 1060 | 1060 |

Example 34 illustrates the pH stability of an embodiment of the present disclosure.

The pH of an aqueous solution is a factor to be considered for medications prepared in aqueous liquid forms. Compound 12 is stable at pH 7.0, (up to 7 days) which makes it easily administered to the body. A stability study of the molecule using in pH 7.0 using NMR profiling showed that it is stable up to 7 days.

The pH of an aqueous solution is a factor to be considered for medications prepared in aqueous liquid forms. The potential effect of pH on solubility is a factor for the stability of the medications to be administered. The pH is important for living systems. The stability at pH 7 allows it to be formulated in a wide range of constituents.

The nature of this stability at a neutral pH is a factor behind its non-toxic nature to human cells. Compound 12 has CC₅₀>200 μM against transformed cells, which cells tend to be more sensitive to many drug agents. In wound healing and in vivo experiments, indicated that a prolonged, strongly acidic wound environment prevents both wound closure and re-epithelialization while a prolonged alkaline environment did not have any negative impact on wound closure or re-epithelialization. Separately, both in vitro and in vivo studies showed that prolonged acidic conditions significantly increased the expression of IL-1α in fibroblast cultures and in wound fluid, whereas prolonged alkaline conditions did not result in elevated amounts of IL-1α. The latter is Interleukin 1α (IL-1α) is a potent inflammatory cytokine that activates the inflammatory process, and its deregulated signaling causes devastating diseases manifested by severe acute or chronic inflammation. Thus, wound treatment at neutral pH is much preferred.

All references throughout this application, for example patent documents, including issued or granted patents or equivalents and patent application publications, and non-patent literature documents or other source material are hereby incorporated by reference herein in their entireties, as though individually incorporated by reference.

What is claimed is:

1. A compound having the following formula I, or a pharmaceutically acceptable salt thereof: 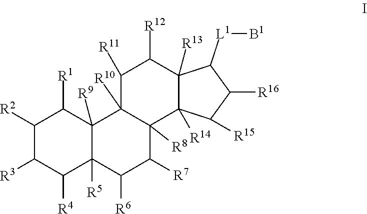
wherein:
R¹-R⁴, R⁶, R⁷, R¹¹, R¹², R¹⁵ and R¹⁶ is each independently a member selected from the group consisting of hydrogen, hydroxyl, a substituted or unsubstituted (C₁-C₁₀) alkyl, a substituted or unsubstituted (C₁-C₁₀) alkylcarboxy, (C₁-C₁₀) hydroxyalkyl, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, a substituted or unsubstituted (C₁-C₁₀) aminoalkylcarboxamido, (C₁-C₁₀) azidoalkyloxy, (C₁-C₁₀) cyanoalkyloxy, (C₁-C₁₀) guanidinoalkyl oxy, and (C₁-C₁₀) guanidinoalkyl carboxy;
R⁵, R⁸, R⁹, R¹⁰, R¹³, and R¹⁴ is each independently a member selected from the group consisting of hydrogen, hydroxyl, a substituted or unsubstituted (C₁-C₁₀) alkyl, (C₁-C₁₀) hydroxyalkyl, (C₁-C₁₀) alkyloxy-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) aminoalkylcarboxy, a substituted or unsubstituted (C₁-C₁₀) aminoalkylaminocarbonyl, (C₁-C₁₀) azidoalkyloxy, (C₁-C₁₀) cyanoalkyloxy, (C₁-C₁₀) guanidinoalkyloxy, and (C₁-C₁₀) guanidinoalkylcarboxy;
L¹ is a linking group; and
B¹ is biotin, desthiobiotin or a biotin mimetic.

2. The compound of claim 1, wherein R³, R⁷ and R¹² is each independently a member selected from the group consisting of a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, and a substituted or unsubstituted (C₁-C₁₀) aminoalkylcarboxamido; and
R⁵, R⁹ and R¹³ is each independently a member selected from the group consisting of hydrogen, hydroxyl, and a substituted or unsubstituted (C₁-C₁₀) alkyl.

3. The compound of claim 1, wherein R³, R⁷ and R¹² is each a substituted or unsubstituted (C₁-C₁₀) amino alkylcarboxy, wherein the amino group is optionally a cationic amine salt; and
R⁵, R⁹ and R¹³ is each independently a hydrogen or a substituted or unsubstituted (C₁-C₃) alkyl.

4. The compound of claim 1, wherein L¹ is -L-Y—Z, wherein L is independently selected from the group consisting of a bond, a substituted or unsubstituted (C₁-C₁₀) alkyl, a substituted or unsubstituted (C₁-C₁₀) alkylcarboxy, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) alkylcarboxamido, a substituted or unsubstituted (C₁-C₁₀) alkylcarboxamido wherein the amino group is substituted, and a substituted or unsubstituted (C₁-C₁₀) alkylaminocarbonyl;
Y is optional and is a member selected from the group consisting of a substituted or unsubstituted (C₁-C₁₀) alkylheterocyclyl (C₁-C₁₀) alkylcarboxamido, a substituted or unsubstituted (C₁-C₁₀) alkylheterocyclyl, a substituted or unsubstituted (C₁-C₁₀) alkylheterocyclyl (C₁-C₁₀) alkylcarboxy, and a substituted or unsubstituted (C₁-C₁₀) alkylheterocyclyl (C₁-C₁₀) alkylaminocarbonyl; and
Z is a member selected from the group consisting of a substituted or unsubstituted C₁-C₃₀ alkylene, a C₁-C₃₀ alkenylene, wherein the alkylene or alkenylene is optionally interrupted by at least one heteroatom, a substituted or unsubstituted (C₁-C₁₀) alkylamino, a substituted or unsubstituted (C₁-C₁₀) alkylcarbonyl, a PEG₁₋₃₀, optionally terminating in a member selected from the group consisting of a bond, —O—, —S—, —NH—, —NHC(O)—, and —C(O) NH—.

5. The compound of claim 1, wherein B¹ is biotin.

6. The compound of claim 1, wherein the compound is a member selected from the group consisting of:

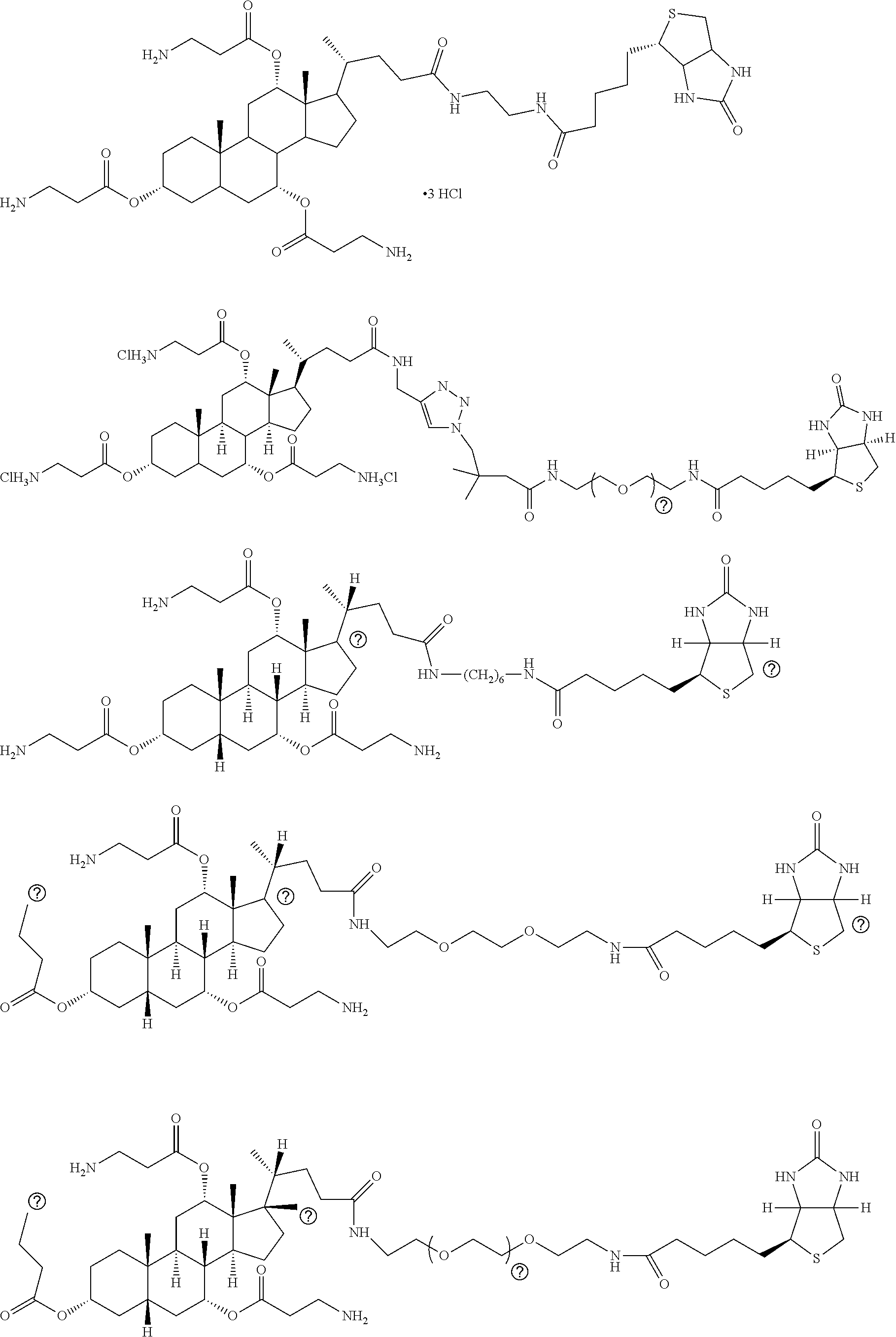

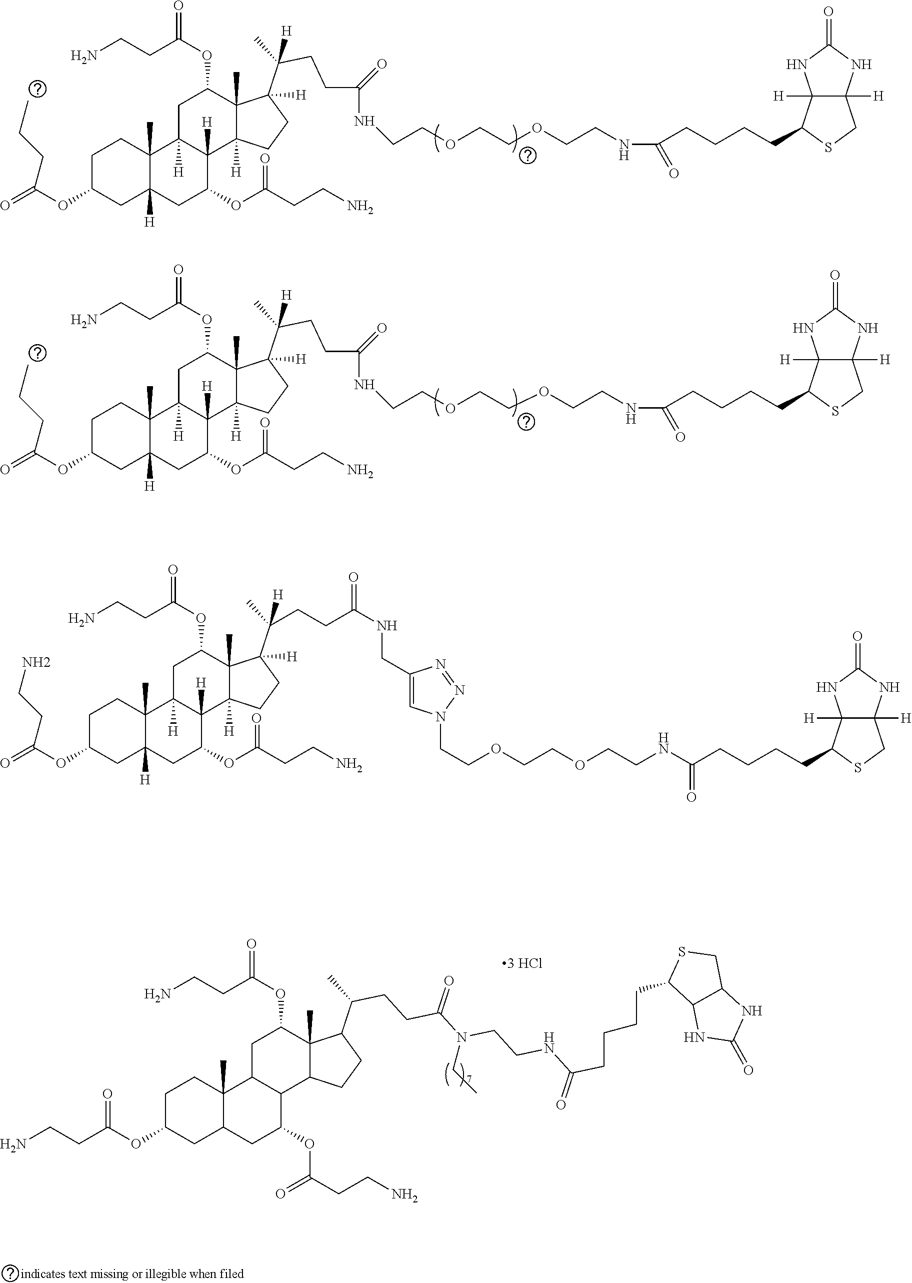

7. The compound of claim 1, wherein the B¹ is desthiobiotin.

8. The compound of claim 1, wherein the compound is a member selected from the group consisting of:

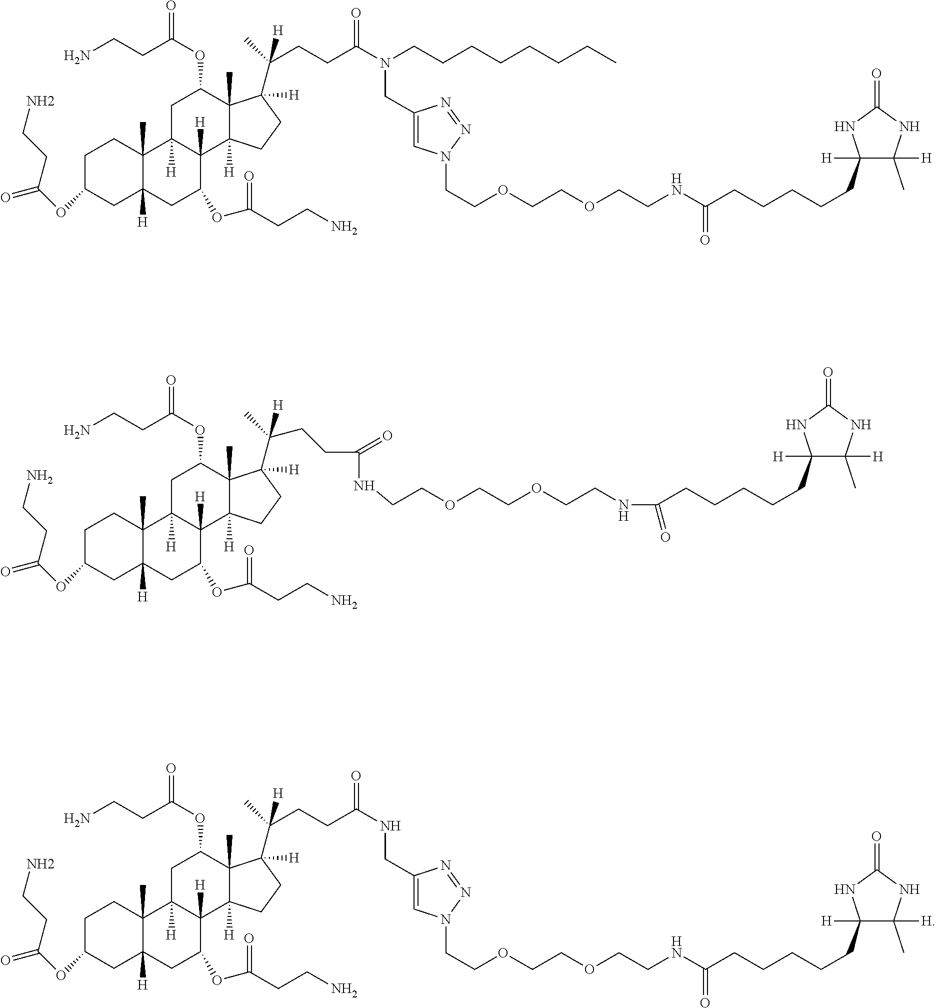

9. The compound of claim 1, wherein B¹ is a biotin memetic.

10. The compound of claim 9, wherein the biotin memetic compound is a member selected from the group consisting of:

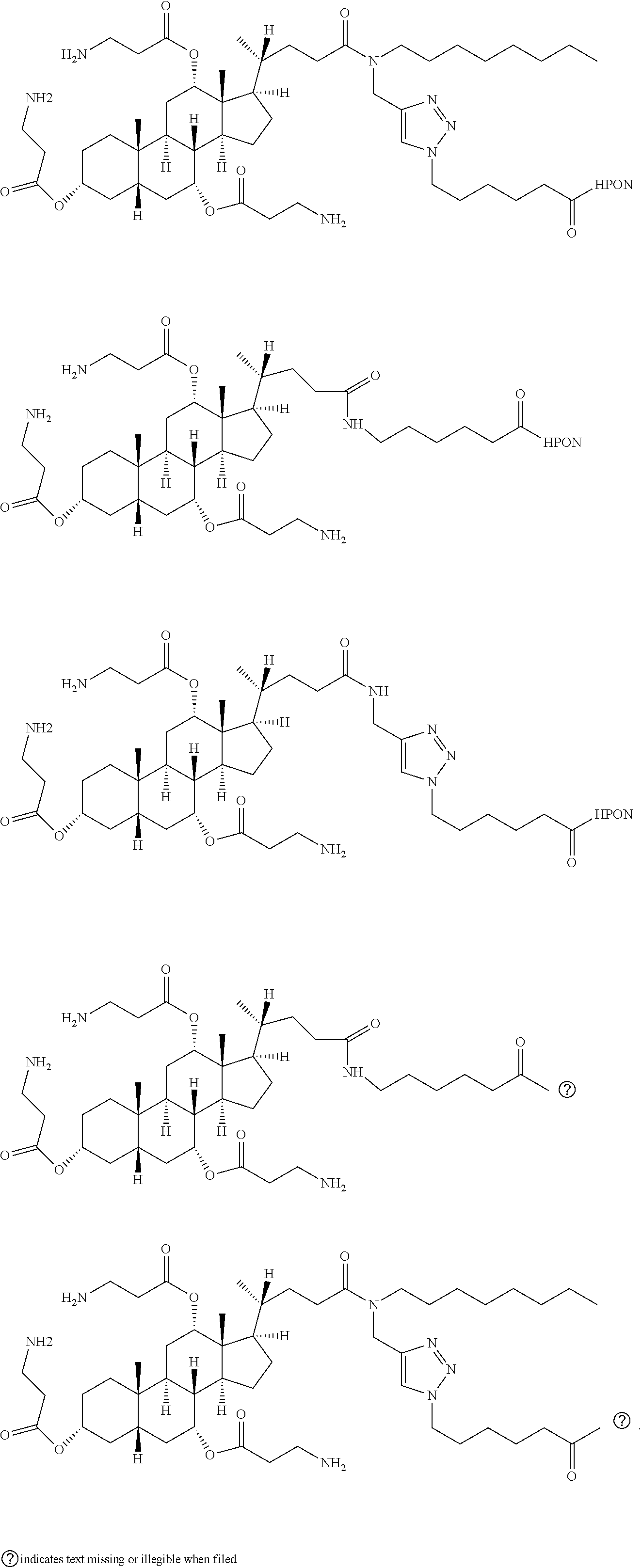

11. The compound of claim 1, wherein the compound has a therapeutic or a prophylactic index of greater than 3, or alternatively, at least 15.

12. A method for treating a microorganism infection or retarding the spread of the microorganism in a subject, the method comprising:
contacting the microorganism with an antimicrobial amount of a compound having formula I or a pharmaceutically acceptable salt thereof: 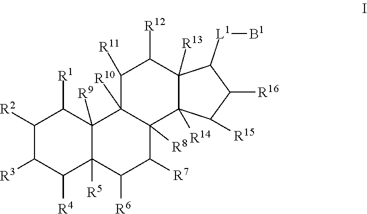
wherein:
R¹-R⁴, R⁶, R⁷, R¹¹, R¹², R¹⁵ and R¹⁶ is each independently a member selected from the group consisting of hydrogen, hydroxyl, a substituted or unsubstituted (C₁-C₁₀) alkyl, a substituted or unsubstituted (C₁-C₁₀) alkyl, a substituted or unsubstituted (C₁-C₁₀) alkylcarboxy, (C₁-C₁₀) hydroxyalkyl, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, a substituted or unsubstituted (C₁-C₁₀) aminoalkylcarboxamido, (C₁-C₁₀) azidoalkyloxy, (C₁-C₁₀) cyanoalkyloxy, (C₁-C₁₀) guanidinoalkyl oxy, and (C₁-C₁₀) guanidinoalkyl carboxy;
R⁵, R⁸, R⁹, R¹⁰, R¹³, and R¹⁴ is each independently a member selected from the group consisting of hydrogen, hydroxyl, a substituted or unsubstituted (C₁-C₁₀) alkyl, (C₁-C₁₀) hydroxyalkyl, (C₁-C₁₀) alkyloxy-(C₁-C₁₀) alkyl, a substituted or unsubstituted (C₁-C₁₀) aminoalkyl, a substituted or unsubstituted aryl, (C₁-C₁₀) haloalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) aminoalkylcarboxy, a substituted or unsubstituted (C₁-C₁₀) aminoalkylaminocarbonyl, (C₁-C₁₀) azidoalkyloxy, (C₁-C₁₀) cyanoalkyloxy, (C₁-C₁₀) guanidinoalkyloxy, and (C₁-C₁₀) guanidinoalkylcarboxy;
L¹ is a linking group; and
B¹ is biotin, desthiobiotin or a biotin memetic, wherein the compound of formula I kills or slows the spread of the microorganism.

13. The method of claim 12, wherein R³, R⁷ and R¹² is each independently a member selected from the group consisting of a substituted or unsubstituted (C₁-C₁₀) aminoalkyloxy, a substituted or unsubstituted (C₁-C₁₀) amino alkylcarboxy, a substituted or unsubstituted (C₁-C₁₀) aminoalkylaminocarbonyl, and a substituted or unsubstituted (C₁-C₁₀) aminoalkylcarboxamido; and
R⁵, R⁹ and R¹³ is each independently a member selected from the group consisting of hydrogen, hydroxyl, and a substituted or unsubstituted (C₁-C₁₀) alkyl.

14. The method of claim 12, wherein R³, R⁷ and R¹² is each a substituted or unsubstituted (C₁-C₁₀) amino alkylcarboxy, wherein the amino group is optionally a cationic amine salt; and
R⁵, R⁹ and R¹³ is each independently a hydrogen or a substituted or unsubstituted (C₁-C₃) alkyl.

15. The method of claim 12, wherein L¹ is-L-Y—Z, wherein L is independently selected from the group consisting of a bond, a substituted or unsubstituted (C₁-C₁₀) alkyl, a substituted or unsubstituted (C₁-C₁₀) alkylcarboxy, (C₁-C₁₀) alkyloxy-(C₁-C₁₀) alkyl, (C₁-C₁₀) alkylamino-(C₁-C₁₀) alkyl, a substituted or unsubstituted (C₁-C₁₀) aminoalkyl, C₂-C₆ alkenyl, C₂-C₆ alkynyl, oxo, a substituted or unsubstituted (C₁-C₁₀) aminoalkyloxy, a substituted or unsubstituted (C₁-C₁₀) amino alkylcarboxy, a substituted or unsubstituted (C₁-C₁₀) alkylcarboxamido, a substituted or unsubstituted (C₁-C₁₀) alkylcarboxamido wherein the amino group is substituted, and a substituted or unsubstituted (C₁-C₁₀) alkylaminocarbonyl;
Y is optional and is a member selected from the group consisting of a substituted or unsubstituted (C₁-C₁₀) alkylheterocyclyl (C₁-C₁₀) alkylcarboxamido, a substituted or unsubstituted (C₁-C₁₀) alkylheterocyclyl, a substituted or unsubstituted (C₁-C₁₀) alkylheterocyclyl (C₁-C₁₀) alkylcarboxy, and a substituted or unsubstituted (C₁-C₁₀) alkylheterocyclyl (C₁-C₁₀) alkylaminocarbonyl; and
Z is a member selected from the group consisting of a substituted or unsubstituted C₁-C₃₀ alkylene, a C₁-C₃₀ alkenylene, wherein the alkylene or alkenylene is optionally interrupted by at least one heteroatom, a substituted or unsubstituted (C₁-C₁₀) alkylamino, a substituted or unsubstituted (C₁-C₁₀) alkylcarbonyl, a PEG₁₋₃₀, optionally terminating in a member selected from the group consisting of a bond, —O—, —S—, —NH—, —NHC(O)—, and —C(O) NH—.

16. The method of claim 12, wherein BL is biotin.

17. The method of claim 12, wherein the compound is a member selected from the group consisting of:

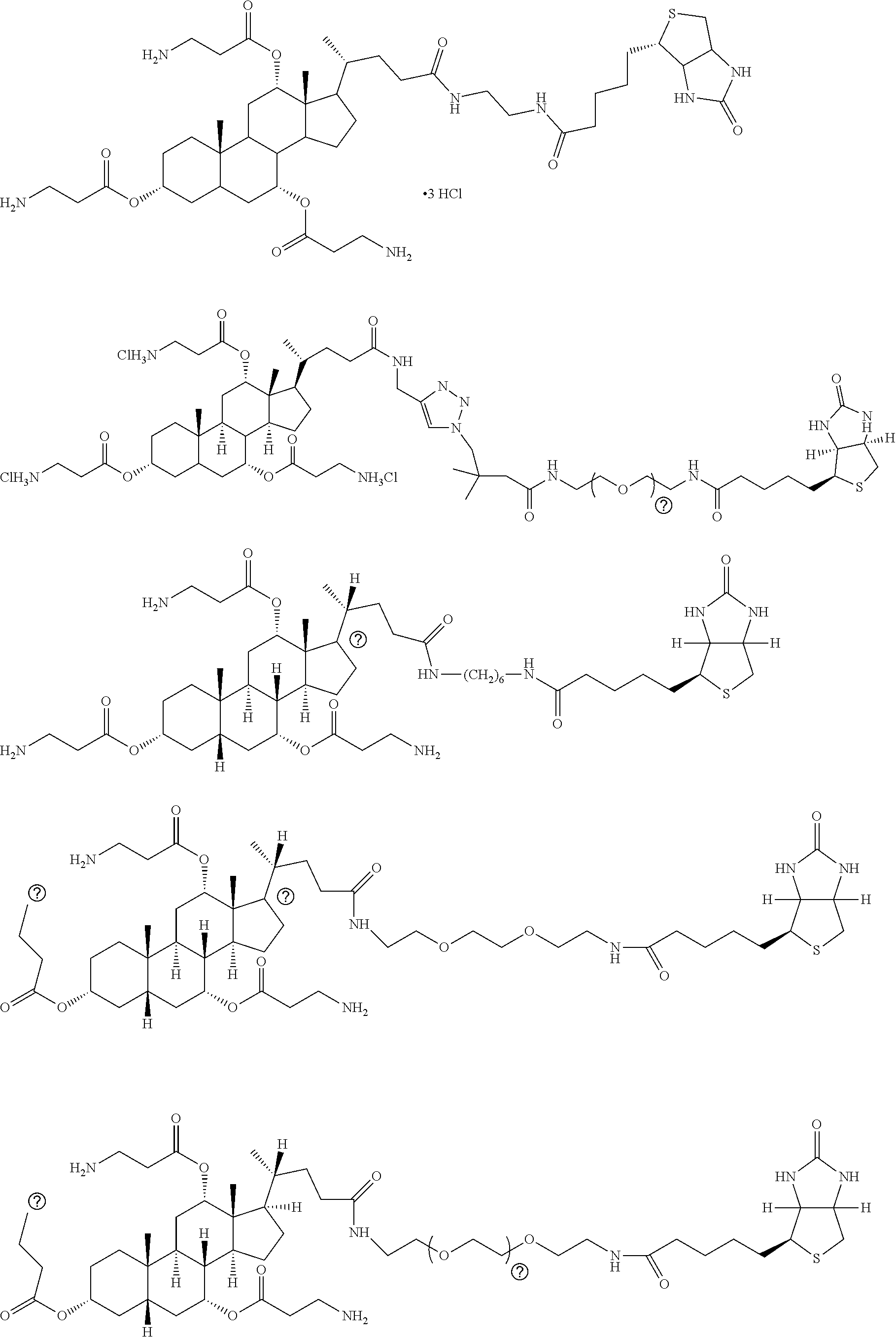

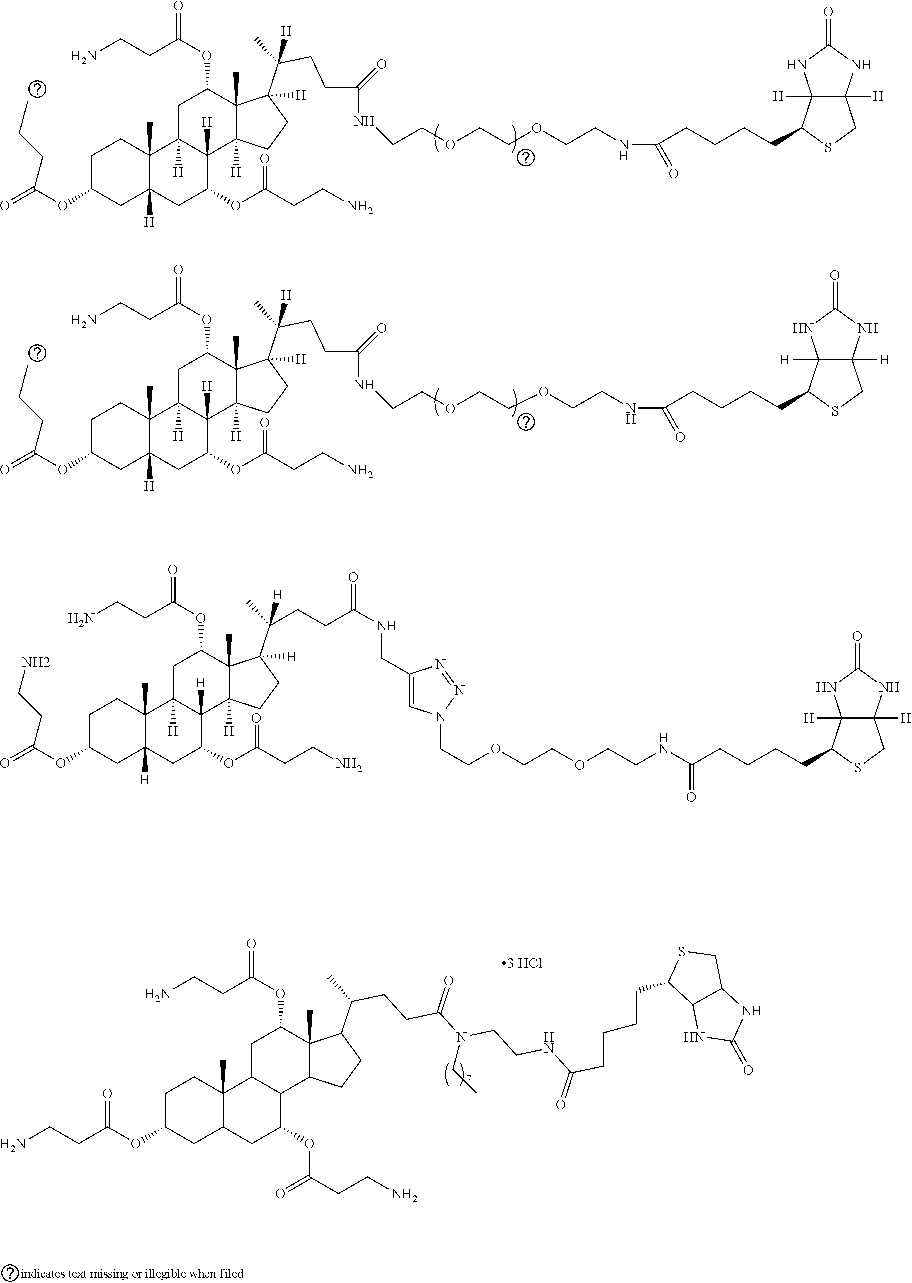

18. The method of claim 12, wherein the compound is a member selected from the group consisting of:

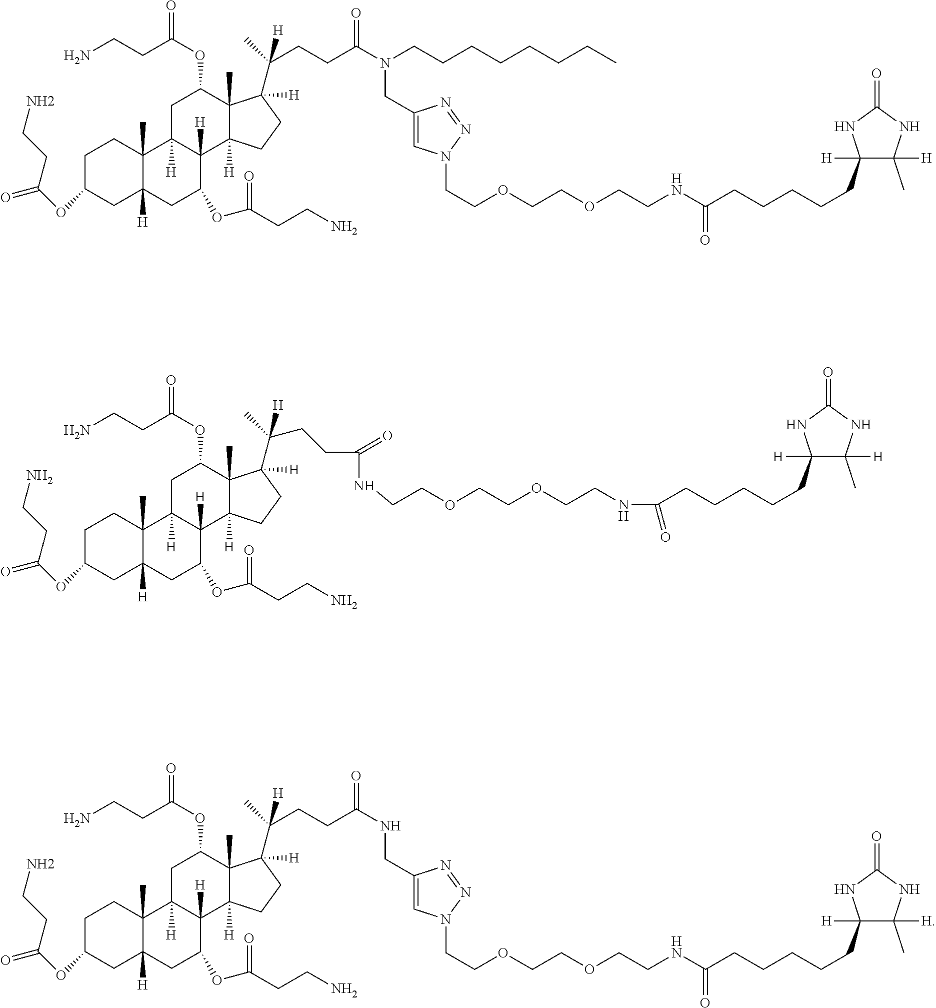

19. The method of any one of claim 12, wherein B¹ is a biotin memetic.

20. The method of claim 19, wherein compound is a member selected from the group consisting of: [chemical structure]
